(19) United States
(12) Patent Application Publication
(10) Pub. No.: US 2026/0183304 A1
(43) Pub. Date: Jul. 2, 2026

(54) COMBINATION OF A TYROSINE KINASE INHIBITOR AND A PRO-INFLAMMATORY AGENT FOR TREATING CANCER

(71) Applicant: MDX Management LLC, La Jolla, CA (US)

(72) Inventors: Yuan LIU, Decatur, GA (US); Lei SHI, Decatur, GA (US); Zhen BIAN, Tucker, GA (US); Harry STYLLI, La Jolla, CA (US)

(21) Appl. No.: 19/126,544

(22) PCT Filed: Nov. 1, 2023
(86) PCT No.: PCT/US2023/078419

§ 371 Date: May 1, 2025

Related U.S. Application Data
(60) Provisional application No. 63/382,003, filed on Nov. 2, 2022.
(60) Provisional application No. 63/491,000, filed on Mar. 17, 2023.
(60) Provisional application No. 63/581,197, filed on Sep. 7, 2023.

(51) Int. Cl. A61K 31/5383; A61K 31/496; A61K 31/5025; A61K 31/506; A61K 31/517; A61K 31/5377; A61K 39/00; A61K 45/06; A61P 35/00; C07K 16/24

(52) U.S. Cl. CPC A61K 31/5383; A61K 31/496; A61K 31/5025; A61K 31/506; A61K 31/517; A61K 31/5377; A61K 45/06; A61P 35/00; C07K 16/241; A61K 2039/505

(57) ABSTRACT

The present application provides methods of treating a cancer in an individual that involves administering to the individual a tyrosine kinase inhibitor and a pro-inflammatory agent (such as a TLR agonist, a STING activator, a radiation therapy, or an immune checkpoint inhibitor). In some cases, the methods involve administering a tyrosine kinase inhibitor when the individual is under an inflammation reaction.

### CROSS-REFERENCE TO RELATED APPLICATIONS

This application claims the benefit of, and priority to U.S. Provisional Application No. 63/382,003, filed Nov. 2, 2022, U.S. Provisional Application No. 63/491,000, filed Mar. 17, 2023, and U.S. Provisional Application No. 63/581,197, filed Sep. 7, 2023, the content of each of which is incorporated herein by reference in their entirety.

### FIELD OF THE INVENTION

The present invention relates to compositions and methods for treating cancer involving administering a tyrosine kinase inhibitor and optionally a pro-inflammatory agent.

### BACKGROUND OF THE INVENTION

In cancers such as solid tumors, intratumoral myeloid leukocytes, including macrophages (i.e., tumor-associated macrophage or TAM) and myeloid-derived suppressive cells (MDSC), play critical roles in controlling the tumor microenvironment (TME) immunosuppression that supports tumor growth and also confers tumor resistance to immunotherapeutic treatments. One important mechanism for myeloid leukocytes to assume an immunosuppressive phenotype, or strengthening their immunosuppressive capacity following tumor therapies, is through their cell surface inhibitory receptors (iRs), which upon activation are also driven by their extracellular ligand binding that triggers multi-pathways of negative regulation via their cytoplasmic domain immunoreceptor tyrosine-based inhibitory motifs (ITIMs) that activate SHP-1, the central signal modulator, to dephosphorylate and hence deactivate a number of signal transduction molecules. This diminishes therapeutics-induced anticancer proinflammatory responses (FIG. 1). In solid tumors, essential cell surface iRs, such as SIRPα, the family of Siglecs, the family of LilRBs and PirB, LAIR1, the family of lectin receptors, the family of SLAM receptors, etc., which also show increased expression in the TME with tumor progression to advanced stages, conduce their regulations via activation of SHP-1, which then mediate downstream inhibition.

Given these inhibitory mechanisms elucidated within previous years, pipelines of therapeutic developments aiming to blockade iRs (e.g., anti-LilRB1/2 and anti-SIRPα) and their ligands (e.g., anti-CD47) are being undertaken (3-5). However, these efforts of targeting each iR or its ligand singularly, but not all inhibitory pathways at once, achieve weak-to-partial efficacies in controlling solid tumors.

The disclosures of all publications, patents, patent applications and published patent applications referred to herein are hereby incorporated herein by reference in their entirety.

### BRIEF SUMMARY OF THE INVENTION

The present application in one aspect provides a method of treating a cancer in an individual, comprising administering to the individual a) a tyrosine kinase inhibitor, and b) a pro-inflammatory agent, optionally wherein the method comprises administering the tyrosine kinase inhibitor to the individual intermittently. In some embodiments, the method comprises systemically or locally (e.g., intratumorally) administering the tyrosine kinase inhibitor. In some embodiments, the pro-inflammatory agent comprises an agent selected from the group consisting of a TLR agonist, a STING activator, a radiation therapy, a PAMP/DAMP molecule, a checkpoint inhibitor, a pro-inflammatory cytokine, a pro-inflammatory cell, a cell, a cancer vaccine, a chemotherapeutic agent, a bacteria component, a cancer vaccine, an oncolytic virus, a sound treatment, a magnetic therapy, an electrical treatment, and an electrostatic treatment.

The present application in another aspect provides a method of treating a cancer in an individual, comprising administering to the individual a) a tyrosine kinase inhibitor, and b) a pro-inflammatory agent, wherein the method comprises systemically administering the tyrosine kinase inhibitor. In some embodiments, the method comprises administering the tyrosine kinase inhibitor to the individual intermittently. In some embodiments, the pro-inflammatory agent comprises an agent selected from the group consisting of a TLR agonist, a STING activator, a radiation therapy, a PAMP/DAMP activator, a checkpoint inhibitor, a pro-inflammatory cytokine, a chemotherapeutic agent, a bacteria component, a cancer vaccine, an oncolytic virus, a sound treatment, a magnetic therapy, an electrical treatment, and an electrostatic treatment.

The present application in another aspect provides a method of treating a cancer in an individual, comprising administering to the individual a) a tyrosine kinase inhibitor, and b) a pro-inflammatory agent, and wherein the pro-inflammatory agent comprises an agent selected from the group consisting of a TLR agonist, a STING activator, a PAMP/DAMP activator, a chemotherapy, a pro-inflammatory cytokine, a cancer vaccine, a bacteria component, a sound treatment, a magnetic therapy, an electrical treatment, and an electrostatic treatment. In some embodiments, the method comprises administering the tyrosine kinase inhibitor to the individual intermittently. In some embodiments, the method comprises systemically administering the tyrosine kinase inhibitor.

The present application in another aspect provides a method of treating a cancer in an individual, comprising administering to the individual a tyrosine kinase inhibitor, wherein the individual is under an inflammation reaction or has an ongoing infection. In some embodiments, the method comprises administering the tyrosine kinase inhibitor to the individual intermittently. In some embodiments, the method comprises systemically administering the tyrosine kinase inhibitor. In some embodiments, the method further comprises immune cells.

In some embodiments according to any one of the methods described above, the method comprises administering the tyrosine kinase inhibitor to the individual at an interval of no more than once every three days for at least twice.

In some embodiments according to any one of the methods described above, the method comprises administering the tyrosine kinase inhibitor to the individual for at least two cycles, wherein each cycle has about three to about twenty days.

In some embodiments according to any of the methods described above, the tyrosine kinase inhibitor has a half-life of no more than about 5 days, optionally the tyrosine kinase inhibitor has a half-life of no more than about 3 days.

In some embodiments according to any of the methods described above, the tyrosine kinase inhibitor is effective in inhibiting more than 50% of the tyrosine kinase activity for no more than about 5 days, optionally wherein the tyrosine kinase inhibitor is effective in inhibiting more than 50% of the tyrosine kinase activity for no more than about 3 days.

In some embodiments according to any of the methods described above, the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets a tyrosine kinase or activated tyrosine kinase). In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406.

In some embodiments according to any of the methods described above, the tyrosine kinase inhibitor is administered at least three times. In some embodiments according to any one of the methods described above, the method comprises administrating the tyrosine kinase inhibitor systemically and locally, optionally wherein the method comprises intratumorally administering the tyrosine kinase inhibitor.

In some embodiments according to any of the methods described above, the systemic administration of a tyrosine kinase comprises oral administration, intravenous administration, subcutaneous administration, and/or intraperitoneal administration.

In some embodiments according to any of the methods described above, the pro-inflammatory agent and the tyrosine kinase inhibitor are administered within about 24 hours (e.g., within about 16 hours, 8 hours, 4 hours, 2 hours, 1 hour, or 0.5 hour) of each other.

In some embodiments according to any of the methods described above, the method comprises intratumorally administering the pro-inflammatory agent.

In some embodiments according to any of the methods described above, the method comprises administering the pro-inflammatory agent to a site that is different from the site of the cancer to be treated.

In some embodiments according to any of the methods described above, the pro-inflammatory agent comprises a TLR agonist. In some embodiments, the TLR agonist activates a TLR on a macrophage. In some embodiments, the TLR comprises TLR2, TLR3, TLR4, TLR5, TLR6, TLR7, TLR8, and/or TLR9. In some embodiments, the TLR agonist comprises CpG, polyI:C and/or R848, flagellin (TLR5), zymosan (TLR2/4), radiation therapy produced DAMP such as HMGB1 (TLR2/4), DNA and RNA molecules (TLR3/7/8/9), etc. In some embodiments, the TLR agonist comprises CpG, polyI:C and R848, for example at 1:1:1 ratio.

In some embodiments according to any of the methods described above, the pro-inflammatory agent comprises a bacteria component, optionally the bacteria component comprises lipopolysaccharide (LPS).

In some embodiments according to any of the methods described above, the pro-inflammatory agent comprises a STING activator. In some embodiments, the STING activator comprises 2′3′-cGAMP.

In some embodiments according to any of the methods described above, the pro-inflammatory agent comprises a chemotherapeutic agent. In some embodiments, the chemotherapy comprises azathioprine (AZA).

In some embodiments according to any of the methods described above, the pro-inflammatory agent comprises a pro-inflammatory cytokine. In some embodiments, the pro-inflammatory cytokine comprises IL-1 family cytokines (e.g., IL-1b, IL-18), IL-6, IL-17, TNF family cytokines (e.g., TNFα), and their combination with type I and type II interferons (IFNα, IFNβ and IFNγ).

In some embodiments according to any of the methods described above that applies, the pro-inflammatory agent comprises a radiation therapy. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is non-ablative, insufficient to eliminate tumor (kill all tumor cells).

In some embodiments according to any of the methods described above, the pro-inflammatory agent comprises a checkpoint inhibitor. In some embodiments, the checkpoint inhibitor comprises an anti-PD-L1 antibody, an anti-PD-1 antibody or an anti-CLTA4 antibody.

In some embodiments according to any of the methods described above, the pro-inflammatory agent is administered intermittently.

In some embodiments according to any of the methods described above, the pro-inflammatory agent and the tyrosine kinase inhibitor are administered simultaneously or concurrently.

In some embodiments according to any of the methods described above, the pro-inflammatory agent comprises immune cells. In some embodiments, the immune cells are derived from the same individual. In some embodiments, the immune cells comprise or are macrophages, optionally wherein the macrophages have a proinflammatory (M1) phenotype. In some embodiments, the immune cells are derived from monocytes. In some embodiments, the immune cells express a high level of MHC-I, MHC-II, CD80 and/or CD86. In some embodiments, the immune cells express one or more pro-inflammatory cytokines, optionally wherein the one or more pro-inflammatory cytokines comprise TNFα and/or IL-12. In some embodiments, the immune cells do not express a significant level of TGFβ and/or IL-10. In some embodiments, the immune cells comprise T cells. In some embodiments, the immune cells are engineered to express a chimeric antigen receptor, optionally wherein the chimeric antigen receptor specifically binds to a tumor antigen. In some embodiments, the macrophages are engineered to be deficient in tyrosine kinase expression and/or activation. In some embodiments, the tyrosine kinase inhibitor and the immune cells are administered within 24 hours of each other, optionally wherein the tyrosine kinase inhibitor and the immune cells are administered within 4 hours of each other. In some embodiments, the immune cells are administered simultaneously or concurrently with the tyrosine kinase inhibitor.

In some embodiments according to any of the methods described above, the method further comprises administering (e.g., locally or systemically) to the individual an effective amount of a SHP-1 inhibitor (e.g., TPI-1 or an analog or a derivative thereof). In some embodiments, the SHP-1 is administered simultaneously with the tyrosine kinase inhibitor. In some embodiments, the SHP-1 is administered sequentially (e.g., prior to or after) with the tyrosine kinase inhibitor. In some embodiments, the SHP-1 administration follows the same dosing schedule as the tyrosine kinase inhibitor.

In some embodiments according to any of the methods described above, the method further comprises administering to the individual an agent that reduces systemic inflammation and/or reduces inflammatory cytokine cascade or cytokine storm, including, but not limited to, an anti-TNFα antibody and an anti-IL6 antibody. In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered sequentially (e.g., prior to or after) with the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor. In some embodiments, the administration of the agent that reduces systemic inflammation and/or reduces inflammatory cytokine cascade or cytokine storm follows the same dosing schedule as the tyrosine kinase inhibitor.

In some embodiments according to any of the methods described above, the cancer is a solid tumor.

In some embodiments according to any of the methods described above, the cancer is a hematological cancer.

In some embodiments according to any of the methods described above, the cancer is a late stage cancer.

In some embodiments according to any of the methods described above, the cancer is resistant or refractory to a radiation therapy, a chemotherapeutic agent, and/or a checkpoint inhibitor.

In some embodiments according to any of the methods described above, the individual is a human.

The present application in another aspect provides a composition comprising a tyrosine kinase inhibitor and a pro-inflammatory agent, optionally wherein the pro-inflammatory agent comprises an agent selected from the group consisting of immune cells, a TLR agonist, a STING activator, an agent used in radiation therapy, a PAMP/DAMP activator, a checkpoint inhibitor, a pro-inflammatory cytokine, a chemotherapeutic agent, a bacteria component, a cancer vaccine, an oncolytic virus, and an agent used in sound treatment, a magnetic therapy, an electrical treatment or an electrostatic treatment.

### BRIEF DESCRIPTION OF THE DRAWINGS

FIG. 1 depicts that SHP-1 serves as the “master” signaling mediator downstream of multi-inhibitory receptors on myeloid leukocytes in the tumor microenvironment (TME). The activity of SHP-1 diminishes RT- and immunotherapies-induced proinflammatory pathways and anti-cancer efficacy, and sustains myeloid leukocyte immunosuppressive phenotype. The approach of tyrosine kinase inhibition as an anti-cancer strategy is marked in a circle. Companies and approaches that aim to deplete or blockade individual cell surface inhibitory receptors including SIRPα (SIRPαnt Immunotherapeutics, anti-CD47 Gilead) and anti-SIRPα (Biosion) approaches), Siglec (NextCure), LilRB (Next-IO), SLAMF (BMS), etc. are partially listed.

FIGS. 2A-2E show a sample assay of in vitro studies of RK-20449 and Dasatinib. FIG. 2A shows the in vitro assay system. FIG. 2B shows that RK20449 and Dasatinib dose-dependently diminished macrophage SHP-1 activity induced by αTLR and cancer cell ligation. FIG. 2C shows that RK20449 and Dasatinib depleted αTLR- and cancer cell ligation-induced iR phosphorylation and binding to SHP-1. FIG. 2D and FIG. 2E show that RK-20449 or Dasatinib treatment enabled macrophage to overcome tumor cell-imposed inhibition to unleash proinflammatory phenotypic expression induced by αTLR, demonstrating marked increases in production of proinflammatory cytokines TNFα, IL-6, and CXCL1 (FIG. D) and expression of cell surface antigen presentation machinery (FIG. 2E). The SHI-1 inhibitor TPI-1, which also demonstrated similar effects, was used in parallel experiments.

FIGS. 3A-3G show the effect of RK-20449 or Dasatinib combined with TLR agonists against solid tumors. FIG. 3A and top panel of FIG. 3F show the experimental design. FIG. 3B show the efficacies of tyrosine kinase inhibitor (TKi) treatment alone or in combination with TLR agonists against MC38 colorectal carcinoma. FIG. 3C and FIG. 3F show the efficacies of TKi treatment alone or in combination with TLR agonists against KPC pancreatic ductal adenocarcinoma. FIG. 3D shows dose-dependent effect of TKi against LLC lung cancer. FIG. 3E shows the effect of Dasatinib combination with various TLR agonists treating MC38 colorectal carcinoma. FIG. 3G shows that inhibition of TK reduces tumor angiogenesis. TKi such as Dasatinib inhibits angiogenesis in tumors. This effect is through inhibition of VEGR, a receptor tyrosine kinase.

FIGS. 4A-4B show that Dasatinib and αTLR combination induces anti-tumor T cell immunity. FIG. 4A shows flowcytometry analyses of the frequency of immune cells (CD45+) within the total cell population of tumor dissociates, as well as frequencies of individual immune cell types labeled by specific antibodies. FIG. 4B shows a summary of each immunopopulation dynamics. Dasatinib once combined with αTLR exhibited dose-dependent effect of elevating CD8+ T cells and NK cells and reducing macrophages and MDSC within TME.

FIGS. 5A-5C show efficacies of RK-20449 or Dasatinib combined with Sting activator (FIG. 5A), tumor-focal RT (FIG. 5B), or proinflammatory cytokines (FIG. 5C) for treating MC38 colorectal cancer.

FIGS. 6A-6C show that PD-1/PD-L1 immune checkpoint blockade bolsters efficacies of Dasatinib and αTLR combination therapy. FIG. 6A shows the experimental scheme. FIG. 6B shows luminescence images showing tumor location and sizes. FIG. 6C shows tumor volume changes post-treatments.

FIGS. 7A-7C show in vitro testing of UM-164, R406, piceatannol, Bafetinib, and Ibrutinib for diminishing the TK-iRS-SHP-1 axis in TAM induced by αTLR and cancer cell ligation. Multiple TKis were tested for capability of diminishing macrophage SHP-1 activity induced by αTLR and cancer cell ligation (FIG. 7A), elevating antigen presentation machinery (FIG. 7B), and induction of proinflammatory cytokines (FIG. 7C).

FIGS. 8A-8C show in vivo testing of R406, UM-164, Piceatannol, and SHP inhibitor 3Ac for anti-tumor efficacy combined with αTLR. FIG. 8A shows that R406, but not other inhibitors, combined with αTLR effectively suppressed LLC tumor. FIG. 8B shows TME analyses demonstrating that R406 combined with αTLR induced intratumoral CD8+ T cell expansion. FIG. 8C shows that R406 combined with αTLR induced intratumoral macrophages for antigen presentation.

FIGS. 9A-9C shows anti-cancer effects of TK inhibitors Ponatinib, Bosutinib, Saracatinib & KX2-391. Four TK inhibitors including Ponatinib, Bosutinib, Saracatinib & KX2-391 were tested for capability of diminishing macrophage SHP-1 activity induced by αTLR and cancer cell ligation (FIG. 9A), and the ability of elevating antigen presentation molecule expression otherwise inhibited by cancer cell ligation (FIG. 9B). In vivo anti-tumor efficacies with the combination of αTLR were shown in (FIG. 9C).

FIGS. 10A-10E show synergistic effects of SHP-1 inhibitor TPI-1 and TK inhibitor Dasatinib. FIG. 10A shows experimental design testing treatment against KPC. FIG. 10B shows that KPC tumor volume changes following treatment with αTLR plus TPI-1, or αTLR plus TPI-1 and Dasatinib, versus tumors that received no treatment (NT) and exhibited continuous progression. FIG. 10C shows the results of TME analyses. Compared to the treatment with αTLR plus TPI-1, αTLR plus TPI-1 and Dasatinib further enhanced T cell immunity while reducing PMN infiltration, resulting in increased CD8 (Tc) and CD4 (Th) T cells, moderately increased NK cells, but reduced PMN in the TME followed treatment. FIG. 10D shows experimental design testing treatment against MC38. FIG. 10E shows MC38 tumor volume changes following treatment with αTLR plus TPI-1, αTLR plus Dasatinib, or αTLR plus TPI-1 and Dasatinib, versus tumors that received no treatment (NT) and exhibited continuous progression.

FIGS. 11A-11G show that anti-TNFα mAb curbs down systemic inflammation and reduces adverse toxicity. FIG. 11A shows the experimental design. Mice with established MC38 colorectal carcinoma (200-400 mm³) were treated with αTLR, TPI-1 and Dasatinib (s.c.), without or with additional treatment with anti-TNFα mAb or anti-IL-6 mAb (150 μg, i.p.). The treatment was repeated once (d1 and d2). Tumor volume changes were recorded, and tumor TMEs were analyzed for immune infiltrates on day 6 post treatments. FIG. 11B shows tumor volume changes following various treatments. FIG. 11C and FIG. 11D show the results of TME analyses. Anti-TNFα mAb or anti-IL-6 mAb treatment did not affect αTLR/TPI-1/Dasatinib therapy-induced increases in CD8 T cells (Tc) and NK cells, as well as reduction of macrophages and MDSC in the TME. FIG. 11E shows that treating mice with anti-TNFα mAb, but not anti-IL-6 mAb, largely diminished the induction of inflammatory cytokines (TNFα, IL-6, IL-1β, IL-10, IFNα, and IFNγ) associated with the αTLR/TPI-1/Dasatinib combination therapy. FIG. 11F shows that Anti-TNFα treatment also markedly reduced monocyte and PMN chemokines CCL2, CCL5 and CXCL1 in circulation, while without reducing CXCL10 that is essential for T cell trafficking. FIG. 11G shows that anti-TNFα treatment protected mice from developing splenomegaly and intestinal inflammation that were commonly associated with αTLR/TPI-1/Dasatinib therapy.

FIG. 12 shows the proinflammatory stimuli (TLR agonists, proinflammatory cytokines IL-1β, IL-6, IL-12, IL-17, IL-18, TNFα, IFNγ, etc., and cancer therapies) induce SIRPα ITIMs phosphorylation and exclusive association of SHP-1 (not SHP-2).

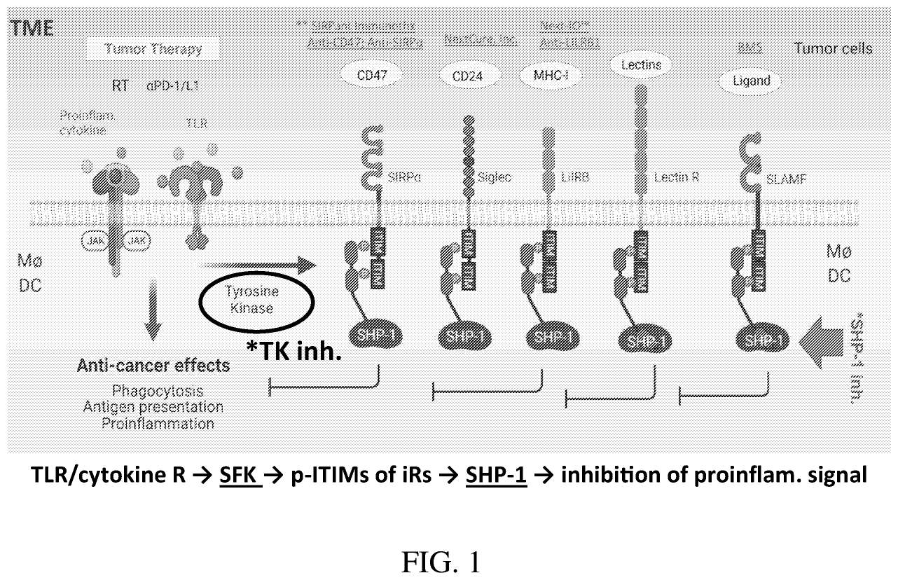

### DETAILED DESCRIPTION OF THE INVENTION

The present application in one aspect provides methods of treating a cancer in an individual, comprising administering to the individual a tyrosine kinase inhibitor, wherein the individual a) has been subject to, is being subject to, or is about to be subject to a pro-inflammatory agent, or b) is under an inflammation reaction or has an ongoing infection. The present application in another aspect provides methods of treating a cancer in an individual, comprising administering to the individual monocytes or macrophages deficient in tyrosine kinase expression or activation, and wherein individual a) has been subject to, is being subject to, or is about to be subject to a pro-inflammatory agent, or b) is under an inflammation reaction or has an ongoing infection. In some embodiments, the tyrosine kinase inhibitor is administered systemically. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the pro-inflammatory agent comprises an agent selected from the group consisting of a TLR agonist, a STING activator, a radiation therapy, a PAMP/DAMP activator, a checkpoint inhibitor, a pro-inflammatory cytokine, a chemotherapeutic agent, a bacteria component, a cancer vaccine, an oncolytic virus, a radiation therapy, a sound treatment, a magnetic therapy, an electrical treatment, and an electrostatic treatment. Further combination therapy methods are provided.

The present application is at least partly based upon a striking finding that combination of a tyrosine kinase inhibitor, which potentially inhibits the activation of a “master” inhibitory executor SHP-1, with a pro-inflammatory treatment unleash proinflammatory signal transduction in tumor environment, especially working on tumor infiltrating macrophages, leading to drastic reprogramming of the TME and bolstering activation of innate and adaptive immune cells to promote anti-cancer immunity. Specifically, it was found that intratumoral iRs-SHP-1 mediated inhibitory regulations are particularly strong under tumor therapies, as these treatments often induce ITIMs to be hyper-phosphorylated, thereby spurring ‘hyper-activation’ of SHP-1, a feedback loop safeguarding tumors from therapeutic damage and inflammatory afront, and also eliciting wound healing response to promote tumor progression. This finding underscores the potential of inhibiting upstream tyrosine kinases, which potentially depletes ITIM phosphorylation and SHP-1 activation, as a combination in tumor immunotherapy in order to achieve efficacies. This approach through nullifying TK activity depletes ITIMs phosphorylation and SHP-1 activation, resulting in cancer treatment.

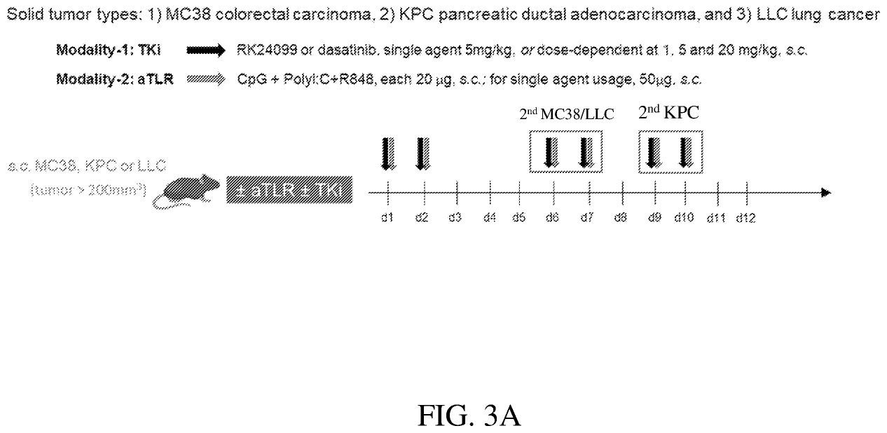

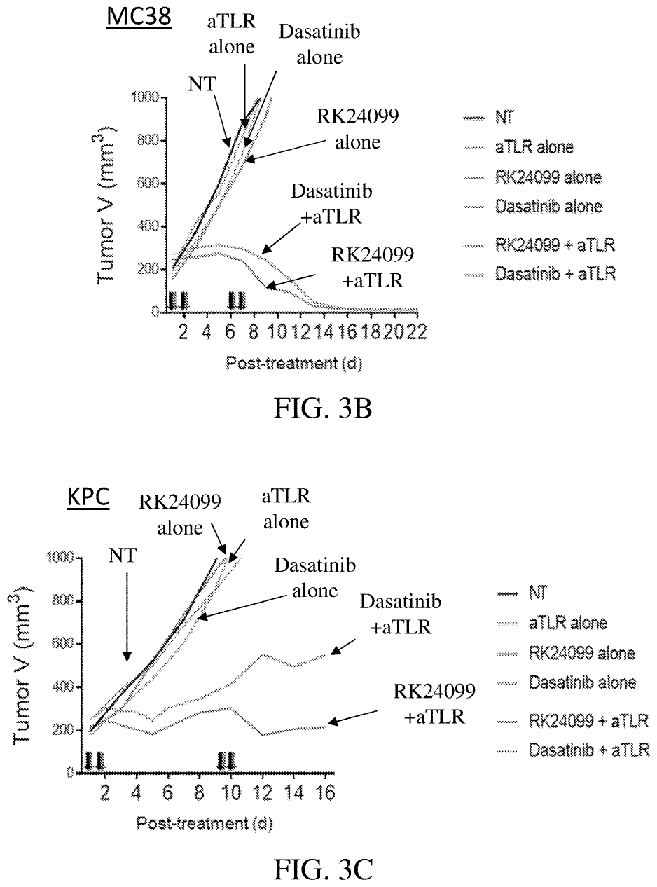

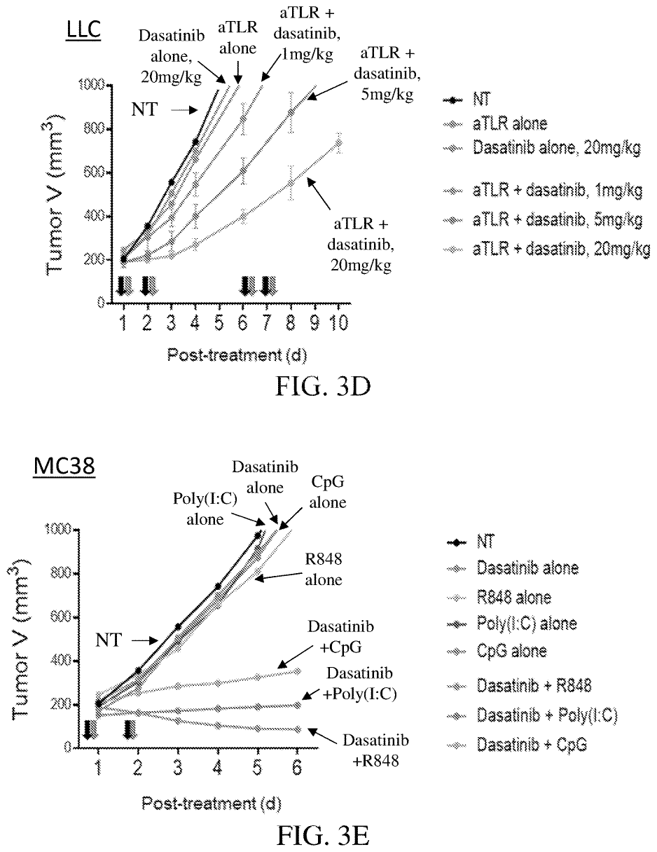

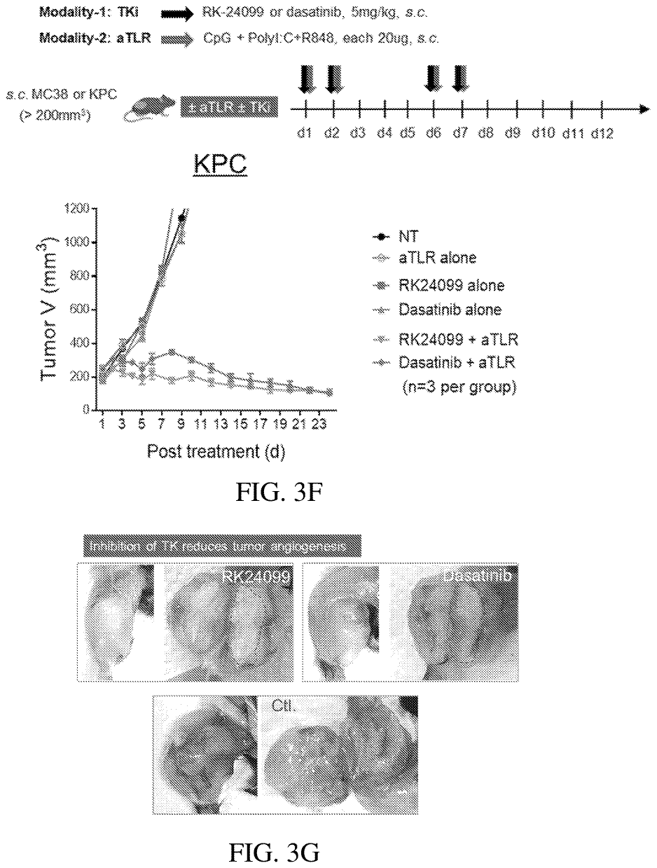

It was demonstrated that combining a tyrosine kinase inhibitor with a pro-inflammatory agent (such as TLR agonists and/or checkpoint inhibitors) achieved striking effects of transforming an immunosuppressive TME into an inflammatory TME, energizing various types of immune cells (such as macrophages, T cells, and B cells), and completely depleting tumors. See e.g., FIGS. 3B-3E, 4A-4B, and 5A-5C.

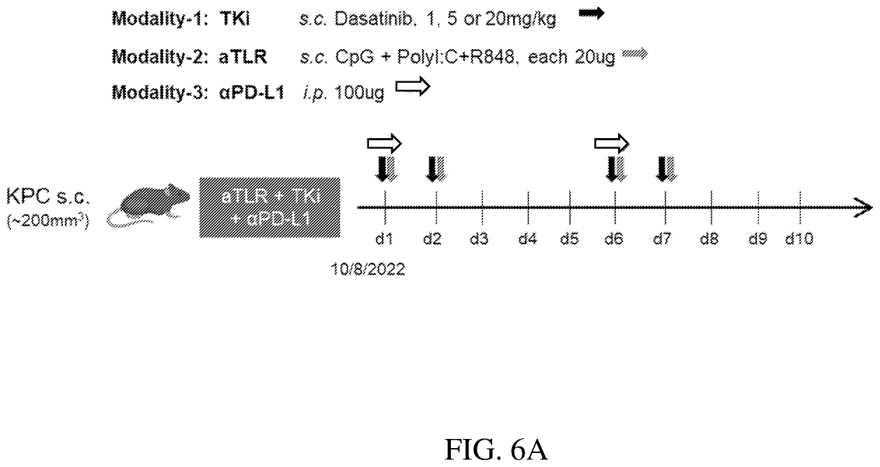

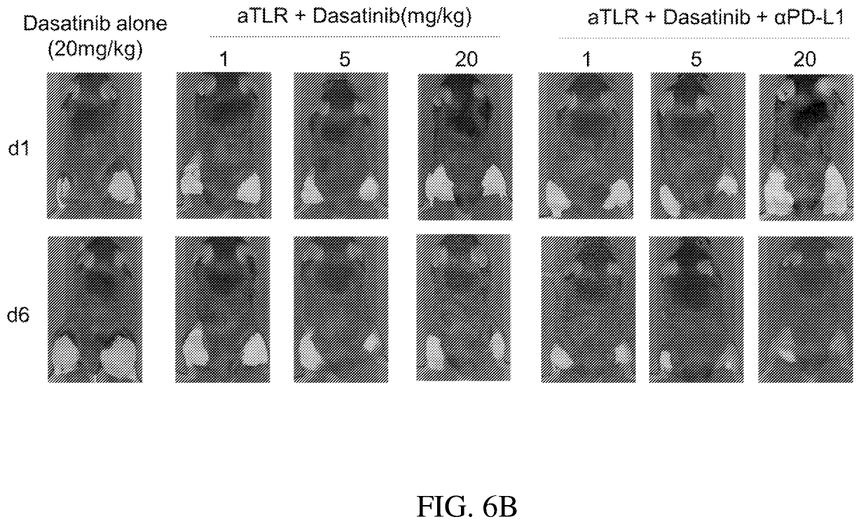

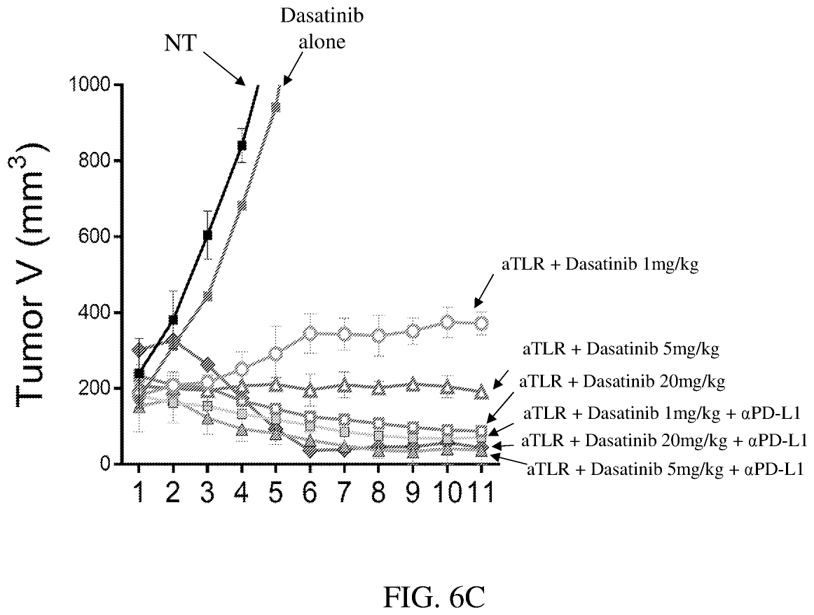

It was also found that tyrosine kinase inhibitor and TLR agonist, when combined with an immune checkpoint inhibitor (e.g., an anti-PD-L1 inhibitor), further bolsters their therapeutic efficacy, accelerating tumor regression. See FIGS. 6A-6C.

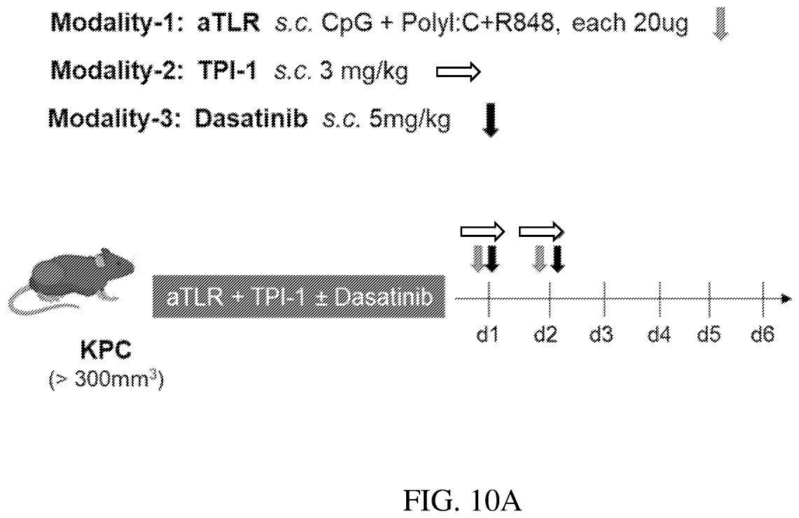

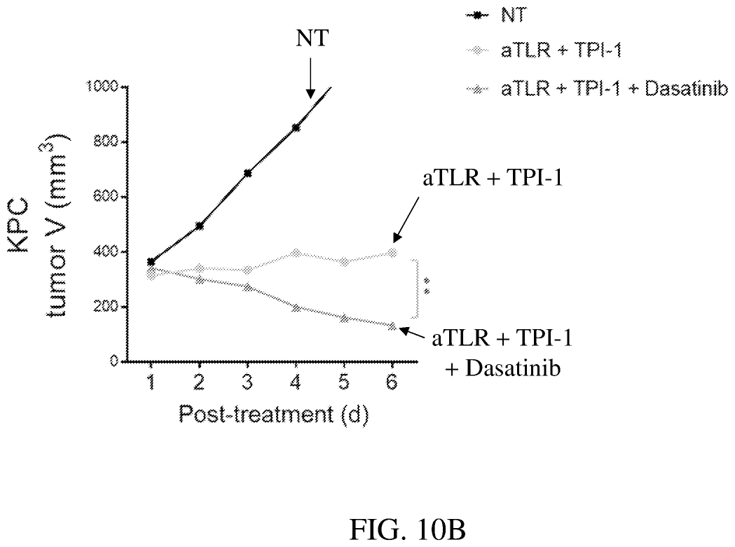

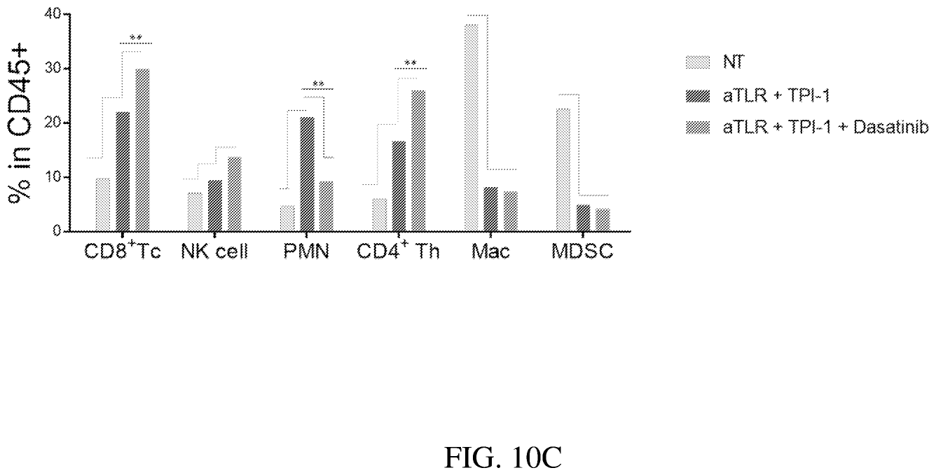

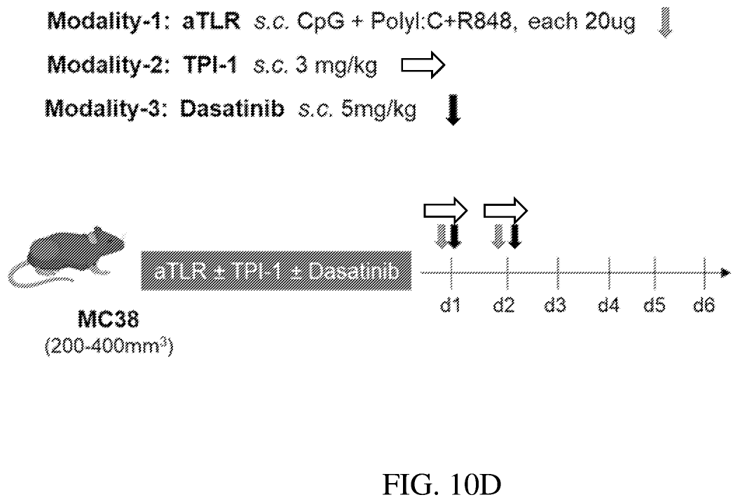

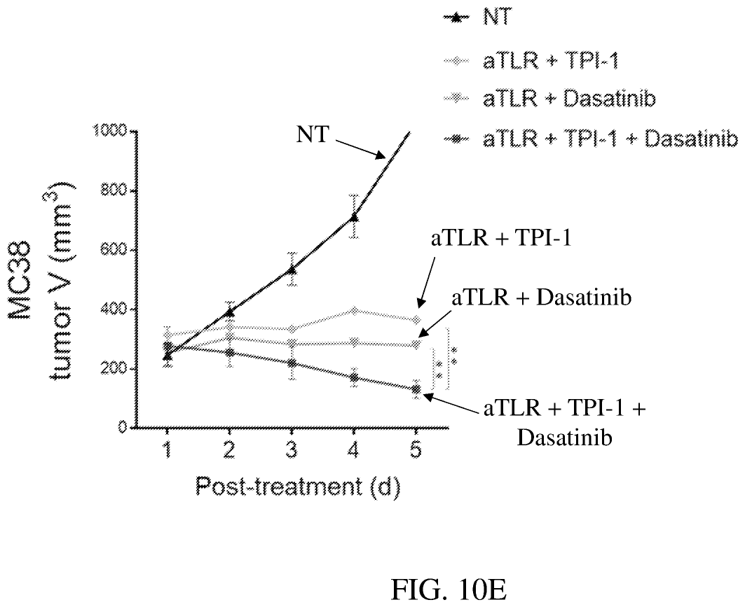

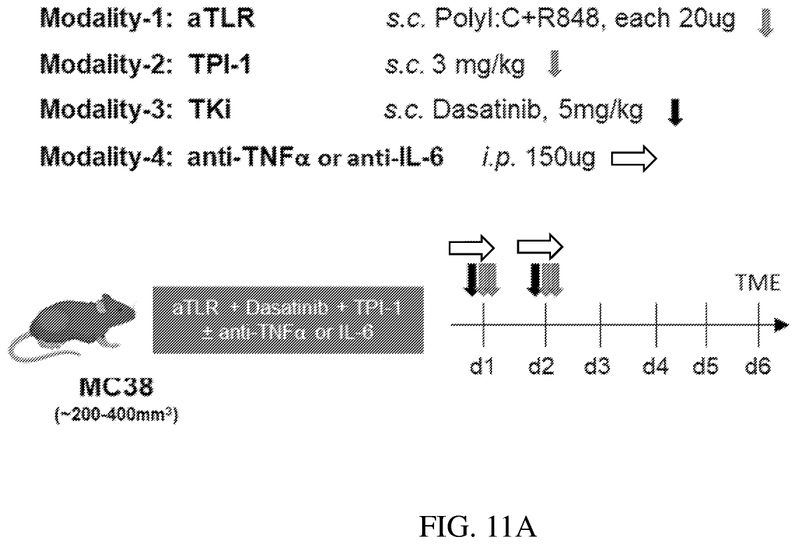

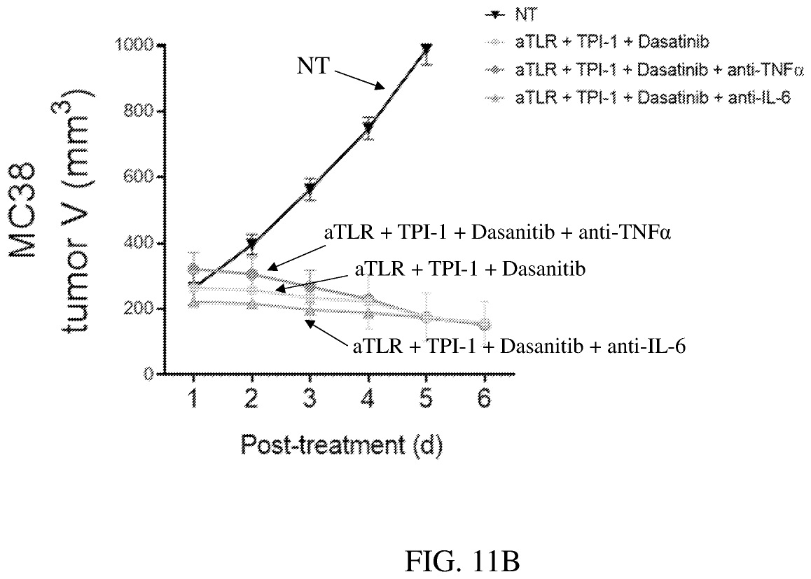

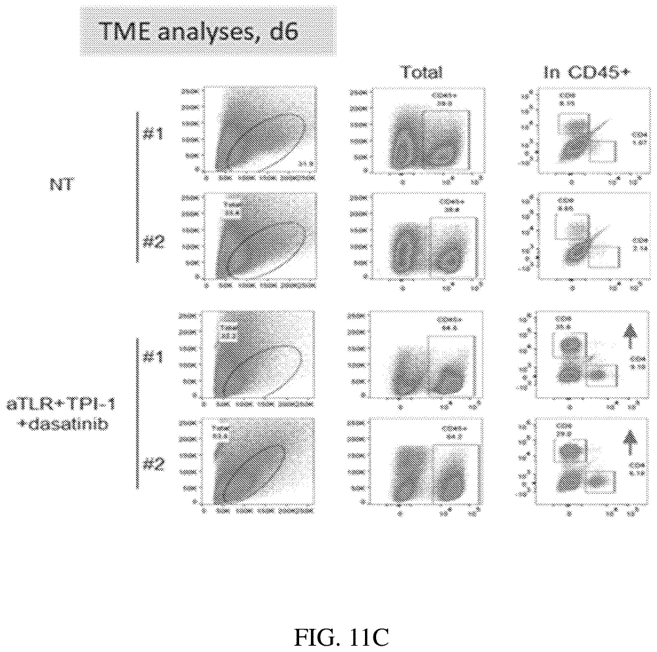

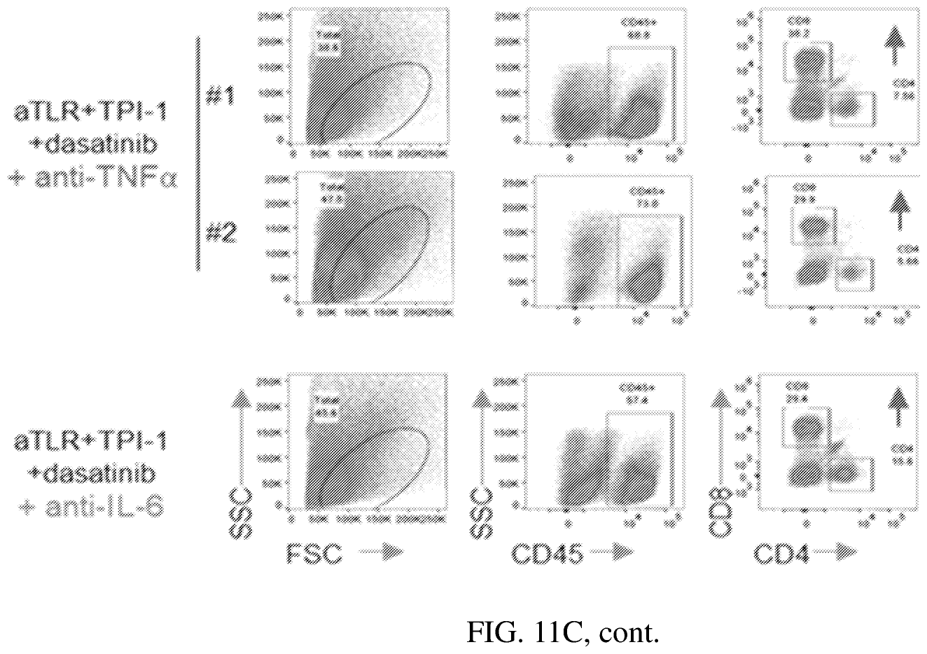

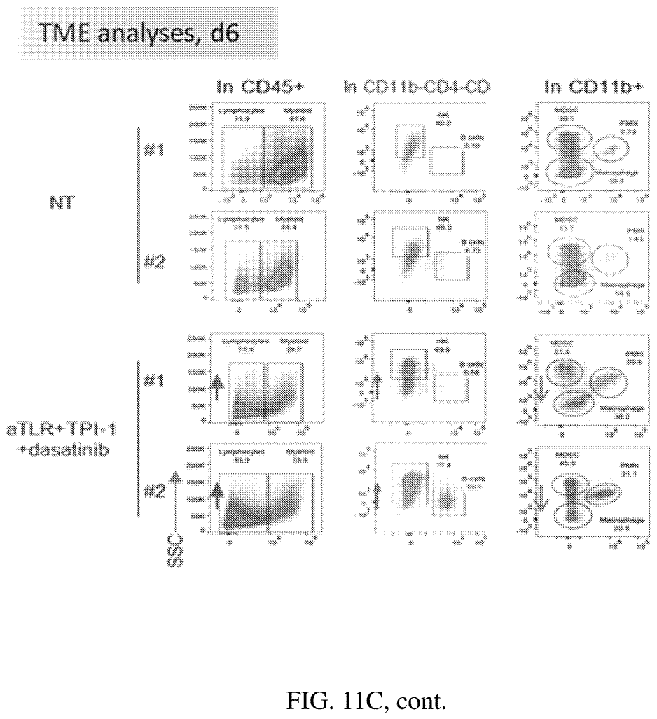

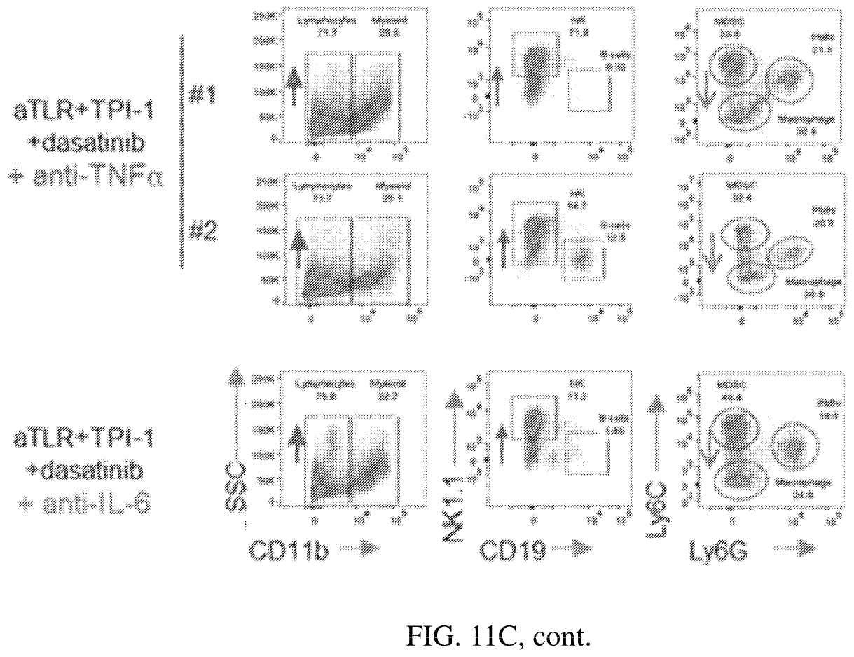

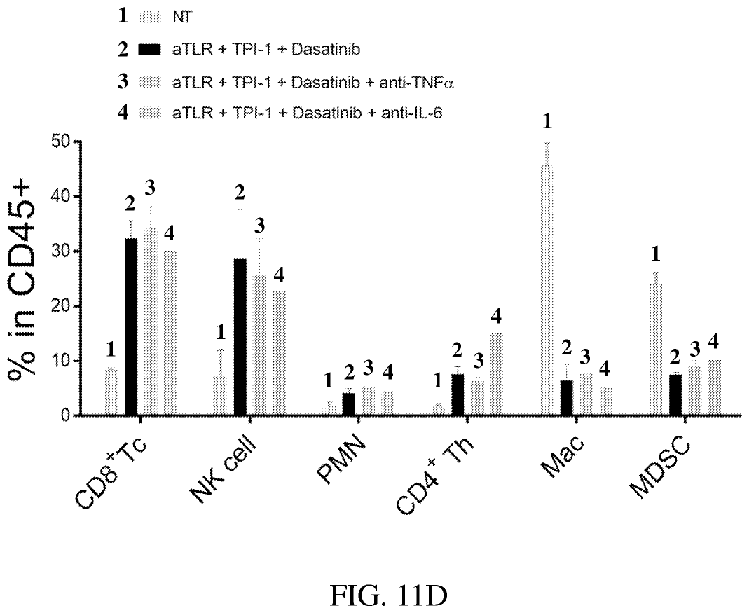

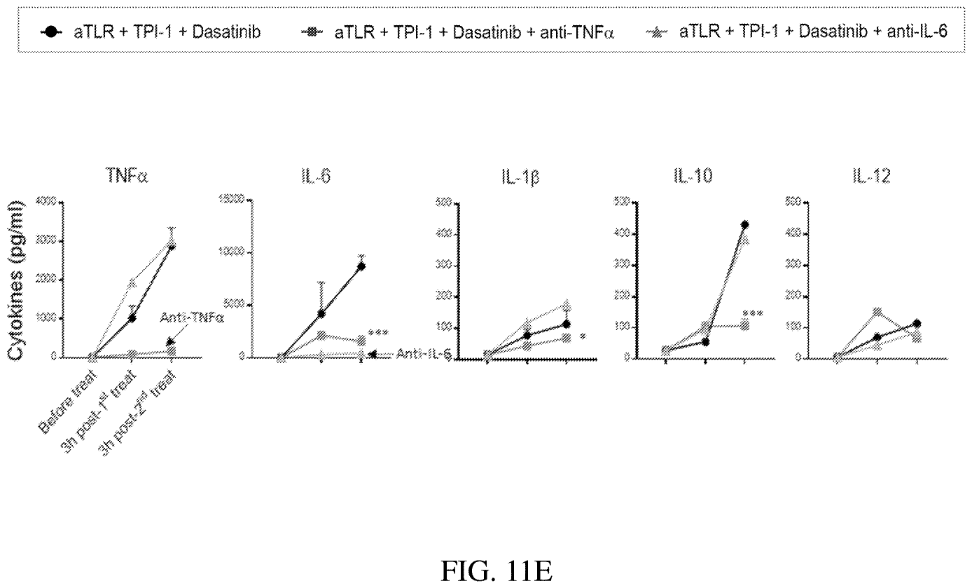

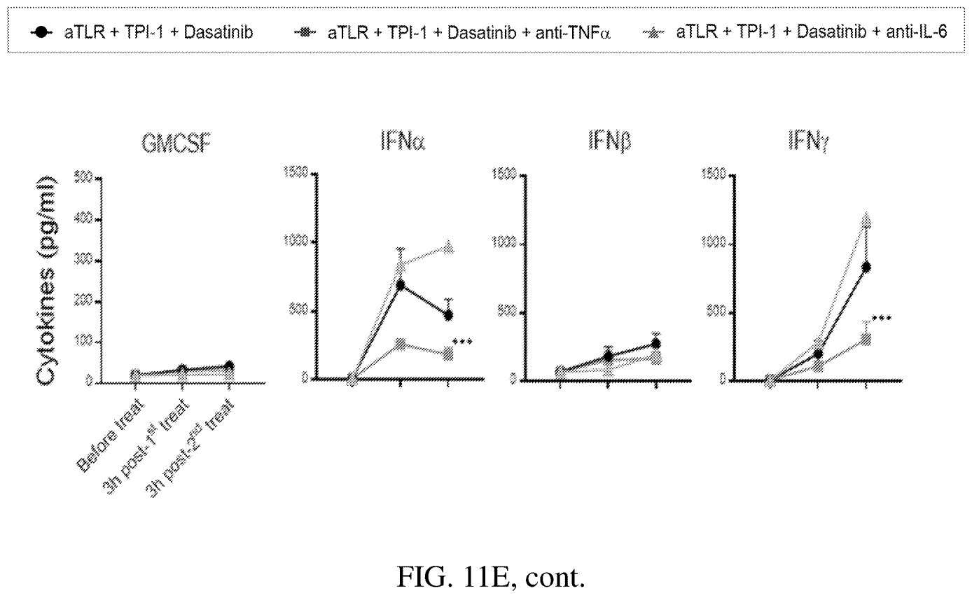

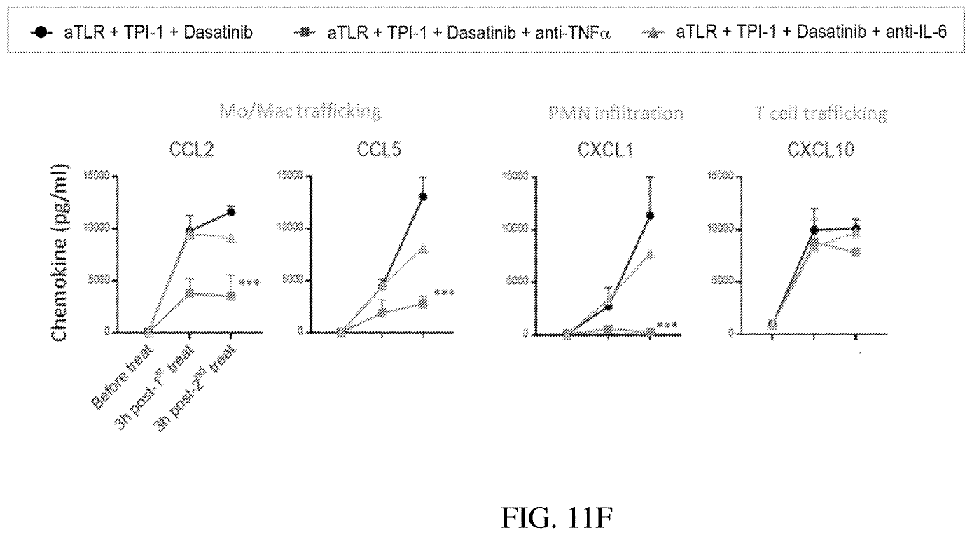

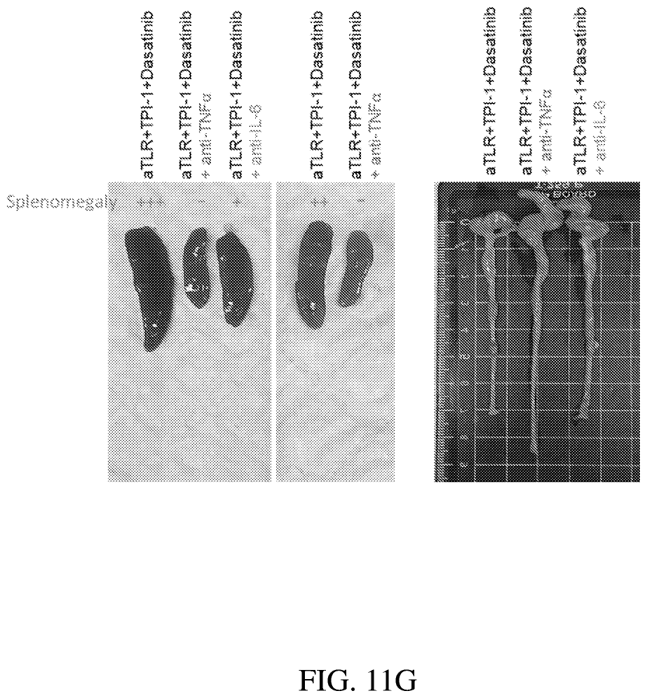

Moreover, tyrosine kinase inhibitor and TLR agonist, when combined with a SHP-1 inhibitor, shows synergistic effects in various tumor models. See FIGS. 10A-10E. Administration of an agent that reduces systemic inflammation (e.g., an anti-TNFα mAb) further curbs down systemic inflammation and reduces adverse toxicity. See FIGS. 11A-11G.

Accordingly, this application provides novel methods that can effectively rewire tumor condition-imposed immunosuppression and license innate and adaptive immunity against cancer, thereby achieving a remarkable anti-tumor efficacy.

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The term “individual,” “subject,” or “patient” is used synonymously herein to describe a mammal, including humans. An individual includes, but is not limited to, human, bovine, horse, feline, canine, rodent, or primate. In some embodiments, the individual is human. In some embodiments, an individual suffers from a disease, such as cancer. In some embodiments, the individual is in need of treatment.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or individual.

As used herein, the term “intermittent” or “intermittently” in the context of dosing refers to a non-continuous dosing. In some cases, “intermittent” dosing refers to a dosing where a) the tyrosine kinase inhibitor is administered less than 12 consecutive days (e.g., less than 11, 10, 9, 8, 7, 6, 5, 4 and 3 days), and b) the tyrosine kinase inhibitor is administered at least two times, and the two administrations are separated by at least one day (i.e., Day 1 and Day 3).

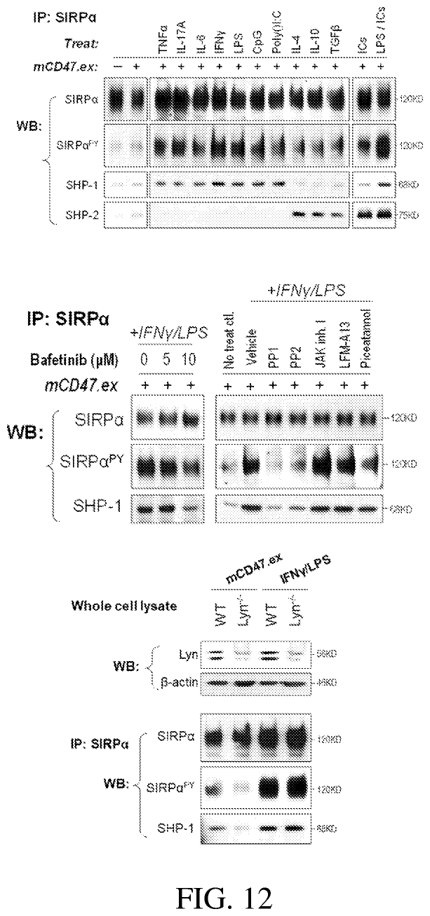

As used herein, the term “cycle” in the context of dosing refers to a time period during which there is at least one administration of a tyrosine kinase inhibitor. Day 1 of a cycle is defined as the day when the first administration of a tyrosine kinase inhibitor happens during that time period. When there are a few daily consecutive administrations of the tyrosine kinase inhibitor, Day 1 of the cycle is defined as the day when first administration among the few daily consecutive administrations happens. The last day of the cycle is defined as the day before the next non-consecutive administration of the tyrosine kinase inhibitor happens. See FIG. 12A and FIG. 14A for exemplary cycles. The cycles do not have to have the same length of time. For example, the first cycle can have five days, and the second cycle have seven days. Each cycle may have different numbers of administrations of the tyrosine kinase inhibitor. For example, the first cycle, which may have five days, may have one administration of the tyrosine kinase inhibitor, and the second cycle, which may have seven days, may have two administrations of the tyrosine kinase inhibitor.

As used herein the term “immunogenic” is the ability to elicit an immune response, e.g., via T-cells, B cells, or both.

As used herein, “treatment” or “treating” is an approach for obtaining beneficial or desired results including clinical results. For purposes of this invention, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread (e.g., metastasis) of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

It should be noted that, as used in the specification and the appended claims, the singular forms “a,” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### II. Methods of Treatment

The present application in one aspect provides methods of treating a cancer by administering a tyrosine kinase inhibitor. In some embodiments, the individual being treated has been subject to, is being subject to, or is about to be subject to a pro-inflammatory agent such as any of those described herein. In some embodiments, the individual is under an inflammation reaction or has an ongoing infection.

In some embodiments, the method comprises administering both a tyrosine kinase inhibitor and a pro-inflammatory agent into the individual. In some embodiments, the tyrosine kinase inhibitor is administered intermittently. In some embodiments, the method comprises systemically administering the tyrosine kinase inhibitor.

In some embodiments, there is provided a method of treating a cancer (e.g., a solid tumor, e.g., a hematological cancer, e.g., a late stage cancer) in an individual, comprising administering to the individual a tyrosine kinase inhibitor, wherein the individual a) has been subject to, is being subject to, or is about to be subject to a pro-inflammatory agent (e.g., a TLR agonist, e.g., a radiation therapy), or b) is under an inflammation reaction or has an ongoing infection, and optionally wherein the tyrosine kinase inhibitor is administered systemically (e.g., intravenously or subcutaneously). In some embodiments, the tyrosine kinase inhibitor is administered intermittently. In some embodiments, the tyrosine kinase inhibitor is administered at an interval of no more than once every two days. In some embodiments, the tyrosine kinase inhibitor is administered no less than two times and no more than 5 times within ten consecutive days (e.g., twice in ten days, three times in ten days, four times in ten days, or five times in ten days). In some embodiments, the tyrosine kinase inhibitor is administered simultaneously with the pro-inflammatory agent. In some embodiments, the tyrosine kinase inhibitor is administered concurrently with the pro-inflammatory agent. In some embodiments, the tyrosine kinase inhibitor and the pro-inflammatory agent are administered sequentially and within 2 weeks (e.g., within 10 days, 7 days, 6 days, 5 days, 4 days, 3 days, 2 days, or the same day). In some embodiments, the tyrosine kinase inhibitor has a half-life of no more than about 10 days (e.g., no more than about 7 days, 5 days, 4 days, or 3 days). In some embodiments, the tyrosine kinase inhibitor is effective in inhibiting more than 50% of the tyrosine kinase activity for no more than about 7 days (e.g., about 5 days, 4 days, or 3 days). In some embodiments, the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets tyrosine kinase or activated tyrosine kinase). In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises locally (e.g., intratumorally) administering the pro-inflammatory agent into the individual. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor. In some embodiments, the TLR agonist is selected from the group consisting of LTA, CpG, PolyI;C, LPS, MPLA, Flagellin, R848, Vesatolimod, Bropirimine, Motolimod, and Loxoribine.

In some embodiments, there is provided a method of treating a cancer (e.g., a solid tumor, e.g., a hematological cancer, e.g., a late stage cancer) in an individual, comprising administering to the individual a tyrosine kinase inhibitor and a pro-inflammatory agent (e.g., a TLR agonist, e.g., a radiation therapy), and wherein the method optionally comprises oral, intravenous or subcutaneous administration of the tyrosine kinase inhibitor, optionally wherein the tyrosine kinase inhibitor is administered intermittently. In some embodiments, the method comprises administering the tyrosine kinase inhibitor to the individual at an interval of no more than once every three days for at least twice. In some embodiments, the tyrosine kinase inhibitor is administered twice (e.g., two executive days) every seven to twenty days. In some embodiments, the tyrosine kinase inhibitor is administered three times (e.g., three executive days) every ten to twenty days. In some embodiments, the tyrosine kinase inhibitor is administered at an interval of no more than once every two days. In some embodiments, the tyrosine kinase inhibitor is administered no less than two times and no more than 5 times within ten consecutive days (e.g., twice in ten days, three times in ten days, four times in ten days, or five times in ten days). In some embodiments, the tyrosine kinase inhibitor is administered simultaneously with the pro-inflammatory agent. In some embodiments, the tyrosine kinase inhibitor is administered concurrently with the pro-inflammatory agent. In some embodiments, the tyrosine kinase inhibitor and the pro-inflammatory agent are administered sequentially and within 2 weeks (e.g., within 10 days, 7 days, 6 days, 5 days, 4 days, 3 days, 2 days, or the same day). In some embodiments, the tyrosine kinase inhibitor has a half-life of no more than about 10 days (e.g., no more than about 7 days, 5 days, 4 days, or 3 days). In some embodiments, the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets tyrosine kinase or activated tyrosine kinase). In some embodiments, the tyrosine kinase inhibitor is a Src inhibitor. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises locally (e.g., intratumorally) administering the pro-inflammatory agent into the individual. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the pro-inflammatory agent. In some embodiments, the pro-inflammatory agent comprises an agent or is selected from the group consisting of R848, 3M-852A, Motolimod, Bropirimine and Vesatolimod. In some embodiments, the pro-inflammatory agent comprises a TLR agonist (e.g., R848) and a pro-inflammatory cytokine (e.g., IFN-gamma). In some embodiments, the TLR agonist is selected from the group consisting of LTA, CpG, PolyI;C, LPS, MPLA, Flagellin, R848, Vesatolimod, Bropirimine, Motolimod, and Loxoribine.

In some embodiments, there is provided a method of treating a cancer (e.g., a solid tumor, e.g., a hematological cancer, e.g., a late stage cancer) in an individual, comprising administering to the individual a tyrosine kinase inhibitor and a pro-inflammatory agent (e.g., a TLR agonist, e.g., a radiation therapy), and wherein the method comprises orally, intravenous or subcutaneous administration of the tyrosine kinase inhibitor, optionally wherein the tyrosine kinase inhibitor is administered intermittently. In some embodiments, the method comprises administering the tyrosine kinase inhibitor to the individual for at least two cycles, further optionally wherein the tyrosine kinase inhibitor is administered for at least once in each cycle and wherein each cycle has about three to about twenty days. In some embodiments, the tyrosine kinase inhibitor is administered for at least twice (e.g., at least two consecutive days) in each cycle. In some embodiments, the tyrosine kinase inhibitor is administered for at least three times (e.g., at least three consecutive days) in each cycle. In some embodiments, the tyrosine kinase inhibitor is administered simultaneously with the pro-inflammatory agent. In some embodiments, the tyrosine kinase inhibitor is administered concurrently with the pro-inflammatory agent. In some embodiments, the tyrosine kinase inhibitor and the pro-inflammatory agent are administered sequentially and within 2 weeks (e.g., within 10 days, 7 days, 6 days, 5 days, 4 days, 3 days, 2 days, or the same day). In some embodiments, the tyrosine kinase inhibitor has a half-life of no more than about 10 days (e.g., no more than about 7 days, 5 days, 4 days, or 3 days). In some embodiments, the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets tyrosine kinase or activated tyrosine kinase). In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises locally (e.g., intratumorally) administering the pro-inflammatory agent into the individual. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the pro-inflammatory agent. In some embodiments, the pro-inflammatory agent comprises an agent or is selected from the group consisting of R848, 3M-852A, Motolimod, Bropirimine and Vesatolimod. In some embodiments, the pro-inflammatory agent comprises a TLR agonist (e.g., R848) and a pro-inflammatory cytokine (e.g., IFN-gamma). In some embodiments, the TLR agonist is selected from the group consisting of LTA, CpG, PolyI;C, LPS, MPLA, Flagellin, R848, Vesatolimod, Bropirimine, Motolimod, and Loxoribine.

In some embodiments, there is provided a method of treating a cancer (e.g., a solid tumor, e.g., a hematological cancer, e.g., a late stage cancer) in an individual, comprising orally, intravenously, subcutaneously and/or intratumorally administering to the individual a tyrosine kinase inhibitor and a pro-inflammatory agent (e.g., a TLR agonist, e.g., a radiation therapy), optionally wherein the tyrosine kinase inhibitor is effective in inhibiting more than 50% of the tyrosine kinase activity for no more than about 5 days, and optionally wherein the method comprises administering the tyrosine kinase inhibitor to the individual at an interval of no more than once every three days for at least twice (e.g., at least 3, 4, 5, or 6 times). In some embodiments, the tyrosine kinase inhibitor is administered intermittently. In some embodiments, the tyrosine kinase inhibitor is administered at an interval of no more than twice every seven to twenty days. In some embodiments, the tyrosine kinase inhibitor is administered at an interval of no more than three times every seven to twenty days. In some embodiments, the tyrosine kinase inhibitor is administered for a period of at least fourteen to twenty days at an interval of about 1-3 times every seven to twenty days. In some embodiments, the tyrosine kinase inhibitor is administered at least about 2, 3, 4, 5, or 6 times in a period of about fourteen to about forty days (e.g., about fourteen to about twenty days). In some embodiments, the tyrosine kinase inhibitor is administered simultaneously with the pro-inflammatory agent. In some embodiments, the tyrosine kinase inhibitor is administered concurrently with the pro-inflammatory agent. In some embodiments, the tyrosine kinase inhibitor and the pro-inflammatory agent are administered sequentially and within 2 weeks (e.g., within 10 days, 7 days, 6 days, 5 days, 4 days, 3 days, 2 days, or the same day). In some embodiments, the tyrosine kinase inhibitor has a half-life of no more than about 10 days (e.g., no more than about 7 days, 5 days, 4 days, or 3 days). In some embodiments, the tyrosine kinase inhibitor is effective in inhibiting more than 50% of the tyrosine kinase activity for no more than about 7 days (e.g., about 5 days, 4 days, or 3 days). In some embodiments, the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets tyrosine kinases or activated tyrosine kinases). In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises locally (e.g., intratumorally) administering the pro-inflammatory agent into the individual. In some embodiments, the tyrosine kinase inhibitor is administered systemically, and the pro-inflammatory agent is administered intratumorally. In some embodiments, the tyrosine kinase inhibitor is administered systemically and intratumorally. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the pro-inflammatory agent. In some embodiments, the pro-inflammatory agent comprises an agent or is selected from the group consisting of R848, 3M-852A, Motolimod, Bropirimine and Vesatolimod. In some embodiments, the pro-inflammatory agent comprises a TLR agonist (e.g., R848) and a pro-inflammatory cytokine (e.g., IFN-gamma). In some embodiments, the TLR agonist is selected from the group consisting of LTA, CpG, PolyI;C, LPS, MPLA, Flagellin, R848, Vesatolimod, Bropirimine, Motolimod, and Loxoribine.

In some embodiments, there is provided a method of treating a cancer (e.g., a solid tumor, e.g., a hematological cancer, e.g., a late stage cancer) in an individual, comprising orally, intravenously, subcutaneously and/or intratumorally administering to the individual a tyrosine kinase inhibitor and a pro-inflammatory agent (e.g., a TLR agonist, e.g., a radiation therapy), wherein the tyrosine kinase inhibitor is effective in inhibiting more than 50% of the tyrosine kinase activity for no more than about 5 days (e.g., for no more than 5, 4, or 3 days), and wherein the tyrosine kinase inhibitor is administered intermittently. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered for at least twice (e.g., at least two consecutive days) in each cycle. In some embodiments, the tyrosine kinase inhibitor is administered for at least three times (e.g., at least three consecutive days) in each cycle. In some embodiments, the tyrosine kinase inhibitor is administered simultaneously with the pro-inflammatory agent. In some embodiments, the tyrosine kinase inhibitor is administered concurrently with the pro-inflammatory agent. In some embodiments, the tyrosine kinase inhibitor and the pro-inflammatory agent are administered sequentially and within 2 weeks (e.g., within 10 days, 7 days, 6 days, 5 days, 4 days, 3 days, 2 days, or the same day). In some embodiments, the tyrosine kinase inhibitor has a half-life of no more than about 10 days (e.g., no more than about 7 days, 5 days, 4 days, or 3 days). In some embodiments, the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets tyrosine kinase or activated tyrosine kinases). In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises locally (e.g., intratumorally) administering the pro-inflammatory agent into the individual. In some embodiments, the tyrosine kinase inhibitor is administered systemically, and the pro-inflammatory agent is administered intratumorally. In some embodiments, the tyrosine kinase inhibitor is administered systemically and intratumorally. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the pro-inflammatory agent. In some embodiments, the pro-inflammatory agent comprises an agent or is selected from the group consisting of R848, 3M-852A, Motolimod, Bropirimine and Vesatolimod. In some embodiments, the pro-inflammatory agent comprises a TLR agonist (e.g., R848) and a pro-inflammatory cytokine (e.g., IFN-gamma). In some embodiments, the TLR agonist is selected from the group consisting of LTA, CpG, PolyI;C, LPS, MPLA, Flagellin, R848, Vesatolimod, Bropirimine, Motolimod, and Loxoribine.

In some embodiments, there is provided a method of treating a cancer (e.g., a solid tumor, e.g., a hematological cancer, e.g., a late stage cancer) in an individual, comprising administering (e.g., orally, intravenously, subcutaneously and/or intratumorally) to the individual a tyrosine kinase inhibitor and immune cells (such as any of the immune cells described herein). In some embodiments, the individual has been subject to, is being subject to, or is about to be subject to a pro-inflammatory agent (e.g., a TLR agonist, e.g., R848, e.g., a radiation therapy). In some embodiments, the individual is under an inflammation reaction or has an ongoing infection. In some embodiments, there is provided a method of treating a cancer (e.g., a solid tumor, e.g., a hematological cancer, e.g., a late stage cancer) in an individual, comprising administering (e.g., intravenously, subcutaneously and/or intratumorally) to the individual a tyrosine kinase inhibitor and a pro-inflammatory agent (e.g., a TLR agonist, e.g., a radiation therapy), and immune cells. In some embodiments, the immune cells are derived from the same individual. In some embodiments, the immune cells comprise monocytes or macrophages. In some embodiments, the immune cells comprise T cells (e.g., CAR-T cells). In some embodiments, the immune cells comprise NK cells (e.g., CAR-NK cells). In some embodiments, the immune cells comprise neutrophils (e.g., CAR-expressing neutrophils cells). In some embodiments, the immune cells comprise antigen presenting cells (APCs). In some embodiments, the immune cells are engineered to express a chimeric receptor that specifically binds to a tumor antigen. In some embodiments, the tyrosine kinase inhibitor is administered intermittently. In some embodiments, the tyrosine kinase inhibitor, the immune cells, and/or the pro-inflammatory agent are administered within 7, 6, 5, 4, 3, 2 or 1 day. In some embodiments, the tyrosine kinase inhibitor and the immune cells are administered within 24 hours (e.g., within 12, 8, 4, 2, or 1 hour, or within 30 minutes) of each other. In some embodiments, the tyrosine kinase inhibitor, the immune cells, and/or the pro-inflammatory agent are administered simultaneously. In some embodiments, the tyrosine kinase inhibitor, the immune cells, and/or the pro-inflammatory agent are administered concurrently. In some embodiments, the tyrosine kinase inhibitor, the immune cells, and/or the pro-inflammatory agent are administered sequentially. In some embodiments, the tyrosine kinase inhibitor is administered systemically, and the pro-inflammatory agent is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the pro-inflammatory agent. In some embodiments, the pro-inflammatory agent comprises an agent or is selected from the group consisting of R848, 3M-852A, Motolimod, Bropirimine and Vesatolimod. In some embodiments, the pro-inflammatory agent comprises a TLR agonist (e.g., R848) and a pro-inflammatory cytokine (e.g., IFN-gamma). In some embodiments, the TLR agonist is selected from the group consisting of LTA, CpG, PolyI;C, LPS, MPLA, Flagellin, R848, Vesatolimod, Bropirimine, Motolimod, and Loxoribine.

In some embodiments, there is provided a method of treating a cancer (e.g., a solid tumor, e.g., a hematological cancer, e.g., a late stage cancer) in an individual, comprising administering to the individual a tyrosine kinase inhibitor and a TLR agonist (e.g., R848), wherein the tyrosine kinase inhibitor is administered at least twice (e.g., at least 3, 4, or 5 times). In some embodiments, the tyrosine kinase inhibitor is administered intermittently. In some embodiments, there is provided a method of treating a cancer (e.g., a solid tumor, e.g., a hematological cancer, e.g., a late stage cancer) in an individual, comprising administering to the individual a tyrosine kinase inhibitor and a TLR agonist, wherein the tyrosine kinase inhibitor and the TLR agonist are administered within 24 hours (e.g., within 12, 8, 4, 2, or 1 hour, or within 30 minutes) of each other. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once (e.g., at least twice or three time) in each cycle and wherein each cycle has about three to about twenty days. In some embodiments, the tyrosine kinase inhibitor is administered systemically (e.g., intravenously or subcutaneously) and/or locally (e.g., intratumorally). In some embodiments, the tyrosine kinase inhibitor and the TLR agonist are administered simultaneously, concurrently or sequentially. In some embodiments, the TLR agonist activates TLR1 or TLR2, optionally wherein the TLR agonist comprises a triacylated lipoprotein, a peptidoglycan, zymosan, and/or Pam3CSK4. In some embodiments, the TLR agonist activates any one of TLR2, TLR3, TLR4, TLR5, and TLR6, optionally wherein the TLR agonist comprises a diacylated lipopeptide, a hot shock protein, HMGB1, uric acid, fibronectin, and/or ECM protein. In some embodiments, the TLR agonist activates TLR2, optionally wherein the TLR agonist comprises Pam3Cys, SMP-105, and/or CBLB612. In some embodiments, the TLR agonist activates TLR3, optionally wherein the TLR agonist comprises dsRNA, Poly I:C, PolyICIC, Poly-IC12U, IPH302, ARNAX, and/or MPLA. In some embodiments, the TLR agonist activates TLR4, optionally wherein the TLR agonist comprises LPS, lipoteichoic acid beta-defensin 2, fibronectin EDA, HMGB1, snapin, tenascin C, OK-432, AS04, and/or GLA-SE. In some embodiments, the TLR agonist activates TLR5, optionally wherein the TLR agonist comprises flagellin, CBLB502, and/or M-VM3. In some embodiments, the TLR agonist activates TLR6. In some embodiments, the TLR agonist activates TLR7 or TLR8, optionally wherein the TLR agonist comprises ssRNA, CpG-A, poly G10, and/or poly G3. In some embodiments, the TLR agonist activates TLR7, optionally wherein the TLR agonist comprises bistriazolyl and/or R848. In some embodiments, the TLR agonist activates TLR8, optionally wherein the TLR agonist comprises VTX1463 and/or R848. In some embodiments, the TLR agonist activates TLR9, optionally wherein the TLR agonist comprises unmethylated CpG DNA, CpG (e.g., CpG-7909, KSK-CpG, CpG-1826), MGN1703, dsSLIM, IMO2055, SD101, and/or ODN M362. In some embodiments, the TLR agonist activates TLR10, optionally wherein the TLR agonist comprises Pam3CSK4. In some embodiments, the TLR agonist activates TLR11, optionally wherein the TLR agonist comprises Toxoplasma gondii profilin. In some embodiments, the TLR agonist activates TLR12. In some embodiments, the TLR agonist activates TLR13, optionally wherein the TLR agonist comprises VSV. In some embodiments, the TLR agonist activates TLR1, TLR2, TLR3, TLR4, TLR7, TLR8, and/or TLR9. In some embodiments, the TLR agonist activates TLR9, TLR4, and TLR7/8. In some embodiments, the TLR agonist comprises CpG, polyI:C, and/or R848. In some embodiments, the TLR agonist comprises CpG, polyI:C, and R848, for example at 1:1:1 ratio. In some embodiments, the tyrosine kinase inhibitor is administered systemically, and the TLR agonist is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the TLR agonist. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the TLR agonist. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the TLR agonist. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the TLR agonist. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the TLR agonist. In some embodiments, the TLR agonist is selected from the group consisting of LTA, CpG, PolyI;C, LPS, MPLA, Flagellin, R848, Vesatolimod, Bropirimine, Motolimod, and Loxoribine.

In some embodiments, there is provided a method of treating a cancer (e.g., a solid tumor, e.g., a hematological cancer, e.g., a late stage cancer) in an individual, comprising administering a tyrosine kinase inhibitor and a TLR agonist (e.g., R848), optionally wherein the TLR agonist activates one or more TLRs selected from the group consisting of TLR9, TLR4, TLR7, and TLR8. In some embodiments, the tyrosine kinase inhibitor and the TLR agonist are administered within the same day. In some embodiments, the tyrosine kinase inhibitor is administered intermittently. In some embodiments, the tyrosine kinase inhibitor and/or the TLR agonist are administered at least twice (e.g., at least three, four, five or six times). In some embodiments, the tyrosine kinase inhibitor and the TLR agonist are administered at least two cycles (e.g., at least three cycles), optionally wherein the tyrosine kinase inhibitor and TLR agonist are administered within the same day for at least two consecutive days (e.g., at least three consecutive days) in each cycle. In some embodiments, each cycle has about seven to about twenty days. In some embodiments, the TLR agonist activates a TLR on a macrophage, optionally wherein the TLR comprises TLR9. In some embodiments, the TLR agonist activates at least two TLRs (e.g., TLR4, TLR7, TLR8, or TLR9). In some embodiments, the TLR agonist activates at least three TLRs (e.g., TLR9, TLR4, and TLR7/8). In some embodiments, the TLR agonist comprises CpG, polyI:C, and/or R848. In some embodiments, the TLR agonist comprises CpG, polyI:C, and R848, for example at 1:1:1 ratio. In some embodiments, the tyrosine kinase inhibitor is administered systemically, and the TLR agonist is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the TLR agonist. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the TLR agonist. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the TLR agonist. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the TLR agonist. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the TLR agonist. In some embodiments, the TLR agonist is selected from the group consisting of LTA, CpG, PolyI;C, LPS, MPLA, Flagellin, R848, Vesatolimod, Bropirimine, Motolimod, and Loxoribine.

In some embodiments, there is provided a method of treating a cancer (e.g., a solid tumor, e.g., a hematological cancer, e.g., a late stage cancer) in an individual, comprising administering to the individual a tyrosine kinase inhibitor and a STING activator (e.g., MSA-2, ADU-S100, or cGAMP), optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and a STING activator (e.g., MSA-2, ADU-S100, or cGAMP), optionally wherein the tyrosine kinase inhibitor and the STING activator are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the tyrosine kinase inhibitor and the STING activator are administered sequentially, simultaneously, or concurrently. In some embodiments, the STING activator is a cyclic-guanosine monophosphate-adenosine monophosphate (cGAMP, e.g., 3′3′ cGAMP, e.g., 2′3′ cGAMP), a bacterial vector (e.g., SYNB1891, STACT-TREX-1), a CDN compounds (e.g., ADU-S100, BI-STING, BMS-986301, GSK532, JNJ-4412, MK-1454, SB11285, 3′3′-cyclic AIMP), a non-CDN small molecule (e.g., ALG-031048, E7755, JNJ-′6196, MK-2118, MSA-1, MSA-2, SNX281, SR-717, TAK676, TTI-10001), a nanovaccine (e.g., PC7A NP, cCAMP-NP, ONM-500) or an antibody-drug conjugate (e.g., XMT-2056, CRD-5500). In some embodiments, the tyrosine kinase inhibitor is administered systemically, and the STING activator is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the STING activator. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the STING activator. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the STING activator. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the STING activator. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the STING activator.

In some embodiments, there is provided a method of treating a cancer (e.g., a solid tumor, e.g., a hematological cancer, e.g., a late stage cancer) in an individual, comprising administering to the individual a tyrosine kinase inhibitor and a radiation therapy, optionally wherein the method comprises administering the tyrosine kinase inhibitor to the individual for at least two cycles, wherein the tyrosine kinase inhibitor is administered for at least once in each cycle and wherein each cycle has about three to about twenty days. In some embodiments, the method comprises administering the tyrosine kinase inhibitor to the individual at an interval of no more than once every three days for at least twice. In some embodiments, days, the tyrosine kinase inhibitor is administered at least three times. In some embodiments, the tyrosine kinase inhibitor is administered systemically (e.g., intravenously, e.g., subcutaneously) and/or locally (e.g., intratumorally). In some embodiments, the tyrosine kinase inhibitor and the radiation therapy are administered within 24 hours (e.g., within 12, 8, 4, 2, or 1 hour, or within 30 minutes) of each other. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is insufficient to kill tumor cells. In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors. In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT). In some embodiments, the radiation therapy is brachytherapy, optionally comprising interstitial brachytherapy, intracavitary brachytherapy, intraluminal radiation therapy, and radioactively tagged molecules given intravenously. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the radiation therapy.

In some embodiments, there is provided a method of treating a cancer (e.g., a solid tumor, e.g., a hematological cancer, e.g., a late stage cancer) in an individual, comprising administering to the individual a tyrosine kinase inhibitor and a radiation therapy, wherein the radiation therapy comprises irradiation at a site that is different from the site of the cancer to be treated. In some embodiments, the tyrosine kinase inhibitor is administered at least twice (at least three, four, five, or six times). In some embodiments, the tyrosine kinase inhibitor is administered intermittently.

In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the tyrosine kinase inhibitor and the radiation therapy are administered within 24 hours (e.g., within 12, 8, 4, 2, or 1 hour, or within 30 minutes) of each other. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is insufficient to kill tumor cells. In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors. In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT). In some embodiments, the radiation therapy is brachytherapy, optionally comprising interstitial brachytherapy, intracavitary brachytherapy, intraluminal radiation therapy, and radioactively tagged molecules given intravenously. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the radiation therapy.

In some embodiments, there is provided a method of treating a cancer (e.g., a solid tumor, e.g., a hematological cancer, e.g., a late stage cancer) in an individual, comprising administering the tyrosine kinase inhibitor and a radiation therapy. In some embodiments, the tyrosine kinase inhibitor is administered intermittently. In some embodiments, the tyrosine kinase inhibitor and the radiation therapy are administered within the same day. In some embodiments, the tyrosine kinase inhibitor and/or the radiation therapy are administered at least twice (e.g., at least three, four, five or six times). In some embodiments, the tyrosine kinase inhibitor and the radiation therapy are administered at least two cycles (e.g., at least three cycles), optionally wherein the tyrosine kinase inhibitor and the radiation therapy are administered within the same day for at least two consecutive days (e.g., at least three consecutive days) in each cycle. In some embodiments, each cycle has about seven to about twenty days. In some embodiments, the tyrosine kinase inhibitor and the radiation therapy are administered within 24 hours (e.g., within 12, 8, 4, 2, or 1 hour, or within 30 minutes) of each other. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is insufficient to kill tumor cells. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the radiation therapy. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the radiation therapy.

In some embodiments, there is provided a method of treating a cancer (e.g., a solid tumor, e.g., a hematological cancer, e.g., a late stage cancer) in an individual, comprising administering to the individual a tyrosine kinase inhibitor and a PAMP/DAMP activator, optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and a PAMP/DAMP activator, optionally wherein the tyrosine kinase inhibitor and the PAMP/DAMP activator are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the pro-inflammatory agent is a PAMP activator. In some embodiments, the PAMP activator is triacyl lipopeptides, LPS, lipoprotein, peptidoglycan, zymosan, lipoteichoic acid, trypanosomal phospholipids, Pam3Cys porins, lipoarabinomannan, double-stranded RNA, poly(I:C), trepanosomal lipids, taxol, Pseudomonas exoenzyme S, RSV F protein, MMTV envelope protein, flagellin, diacyl lipopeptides, single-stranded RNA, imiquimod, single-stranded RNA, resquimod, bacterial/viral DNA, CpG DNA, ureobacteria, or Toxoplasma LPS. In some embodiments, the pro-inflammatory agent is a DAMP activator. In some embodiments, the DAMP activator is defensins, HSP60, HSP70, messenger RNA, low-molecular-weight hyaluronic acid, fibrinogen, fibronectin, fx1-defensin, heparan sulfate, HSP60, HSP70, HSP90, HMGB1, or unmethylated CpG DNA. In some embodiments, the tyrosine kinase inhibitor is administered systemically, and the PAMP/DAMP activator is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the PAMP/DAMP activator. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the PAMP/DAMP activator. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the PAMP/DAMP activator. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the PAMP/DAMP activator. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the PAMP/DAMP activator.

In some embodiments, there is provided a method of treating a cancer (e.g., a solid tumor, e.g., a hematological cancer, e.g., a late stage cancer) in an individual, comprising administering to the individual a tyrosine kinase inhibitor and a checkpoint inhibitor (e.g., an anti-PD-1 agent, an anti-PD-L1 agent, or an anti-CTLA-4 agent), optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and a checkpoint inhibitor (e.g., an anti-PD-1 agent, an anti-PD-L1 agent, or an anti-CTLA-4 agent), wherein the tyrosine kinase inhibitor and the checkpoint inhibitor are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the checkpoint inhibitor targets LAG-3, TIM-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1 (M-CSF)/CSF-1R, IL-1/IL-1R3 (IL-1RAP), IL-8, SEMA4D, Ang-2, CLEVER-1, Axl, or phosphatidylserine. In some embodiments, the checkpoint inhibitor comprises or is lipilimumab, Cemiplimab, Nivolumab, Pembrolizumab, Atezolizumab, Avelumab, Durvalumab, LAG525 (IMP701), REGN3767, BI 754,091, tebotelimab (MGD013), eftilagimod alpha (IMP321), FS118, MBG453, Sym023, TSR-022, MGC018, FPA150, EOS100850, AB928, CPI-006, Monalizumab, COM701, CM24, NEO-201, Defactinib, PF-04136309, MSC-1, Hu5F9-G4 (5F9), ALX148, TTI-662, RRx-001, Lanotuzumab (MCS110), LY3022855, SNDX-6352, Emactuzumab (RG7155), Pexidartinib (PLX3397), CAN04, Canakinumab (ACZ885), BMS-986253, Pepinemab (VX15/2503), Trebananib, FP-1305, Enapotamab vedotin (EnaV), or Bavituximab. In some embodiments, the tyrosine kinase inhibitor is administered systemically, and the checkpoint inhibitor is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the checkpoint inhibitor. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the checkpoint inhibitor. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the checkpoint inhibitor. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the checkpoint inhibitor. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the checkpoint inhibitor.

In some embodiments, there is provided a method of treating a cancer (e.g., a solid tumor, e.g., a hematological cancer, e.g., a late stage cancer) in an individual, comprising administering to the individual a tyrosine kinase inhibitor and a pro-inflammatory cytokine (e.g., IL-1b, IL-18, IL-6, and/or TNFα), optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and a pro-inflammatory cytokine (e.g., IL-1b, IL-18, IL-6, and/or TNFα), wherein the tyrosine kinase inhibitor and the pro-inflammatory cytokine are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the pro-inflammatory cytokine promotes the M1 macrophages. In some embodiments, the pro-inflammatory cytokine comprises or is TNF, IFNγ, and/or GM-CSF. In some embodiments, the pro-inflammatory cytokine comprises IFNγ. In some embodiments, the pro-inflammatory cytokine comprises IL-1. In some embodiments, the pro-inflammatory cytokine comprises TNF-α. In some embodiments, the pro-inflammatory cytokine comprises IL-6. In some embodiments, the tyrosine kinase inhibitor is administered systemically, and the pro-inflammatory cytokine is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the pro-inflammatory cytokine. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the pro-inflammatory cytokine. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the pro-inflammatory cytokine. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the pro-inflammatory cytokine. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the pro-inflammatory cytokine.

In some embodiments, there is provided a method of treating a cancer (e.g., a solid tumor, e.g., a hematological cancer, e.g., a late stage cancer) in an individual, comprising administering to the individual a tyrosine kinase inhibitor and a chemotherapeutic agent (e.g., azathioprine), optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and a chemotherapeutic agent (e.g., azathioprine), wherein the tyrosine kinase inhibitor and the chemotherapy are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the chemotherapeutic agent is an alkylating agent. In some embodiments, the alkylating agent is selected from the group consisting of nitrogen mustard (e.g., endamustine, cyclophosphamide, ifosfamide), nitrosoureas (e.g., carmustine, lomustine), platinum analogs (e.g., carboplatin, cisplatin, oxaliplatin), triazenes (e.g., dacarbazine, procarbazine, temozolamide), alkyl sulfonate (e.g., busulfan), and ethyleneimine (e.g., thiotepa). In some embodiments, the chemotherapeutic agent is an antimetabolite. In some embodiments, the antimetabolite is selected from the group consisting of icytidine analogs (e.g., azacitidine, decitabine, cytarabine, gemcitabine), folate antagonists (e.g., methotrexate, pemetrexed), purine analogs (e.g., cladribine, clofarabine, nelarabine), pyrimidine analogs (e.g., fluorouracil (5-FU), capecitabine (prodrug of 5-FU)). In some embodiments, the chemotherapeutic agent is an antimicrotubular agent. In some embodiments, the antimmicrotubular agent is selected from the group consisting of topoisomerase II inhibitors (e.g., anthracyclines, doxorubicin, daunorubicin, idarubicin, mitoxantrone), topoisomerase I inhibitors (e.g., irinotecan, topotecan), taxanes (e.g., paclitaxel, docetaxel, cabazitaxel), vinca alkaloids (e.g., vinblastine, vincristine, vinorelbine), antibiotics (e.g., actinomycin D, bleomycin, daunomycin). In some embodiments, the chemotherapeutic agent is hydroxyurea, tretinoin, arsenic trioxide, or a proteasome inhibitor (e.g., bortezomib). In some embodiments, the tyrosine kinase inhibitor is administered systemically, and the chemotherapeutic agent is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the chemotherapeutic agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the chemotherapeutic agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the chemotherapeutic agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the chemotherapeutic agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the chemotherapeutic agent.

In some embodiments, there is provided a method of treating a cancer (e.g., a solid tumor, e.g., a hematological cancer, e.g., a late stage cancer) in an individual, comprising administering to the individual a tyrosine kinase inhibitor and a cancer vaccine, optionally wherein the tyrosine kinase inhibitor is administered at least twice. In some embodiments, there is provided a method of treating a cancer (e.g., a solid tumor, e.g., a hematological cancer, e.g., a late stage cancer) in an individual, comprising administering to the individual a tyrosine kinase inhibitor and a cancer vaccine, wherein the tyrosine kinase inhibitor and the cancer vaccine are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the cancer vaccine comprises a cell-based vaccine, a peptide-based vaccine, a viral-based vaccine, and/or a nucleic acid-based vaccine. In some embodiments, the tyrosine kinase inhibitor is administered systemically, and the cancer vaccine is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the cancer vaccine. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the cancer vaccine. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the cancer vaccine. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the cancer vaccine. In some embodiments, the agent that reduces systemic inflammation and/or reduces inflammatory cytokine cascade or cytokine storm (e.g., a TNFα inhibitor, e.g., an anti-TNFα antibody) is administered immediately after e.g., within about any of 5 min, 10 min, 15 min, 30 min, 45 min, 60 min, 1 hr, 2 hrs, or 3 hrs after) the tyrosine kinase inhibitor and/or the cancer vaccine.

In some embodiments, there is provided a method of treating a cancer (e.g., a solid tumor, e.g., a hematological cancer, e.g., a late stage cancer) in an individual, comprising administering to the individual a tyrosine kinase inhibitor and an oncolytic virus, optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and a oncolytic virus, wherein the tyrosine kinase inhibitor and the oncolytic virus are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the oncolytic virus comprises or is an adenovirus (e.g., ONYX-15, LOAd703 virus), a protoparvovirus, a parvovirus (e.g., H-1PV), a vaccinia virus (VACV), a Reovirus (e.g., Reolysin), or a Herpes simplex virus (HSV, e.g., HSV-1, HSV-2, G207, LIBR1, HF10, T-VEC, Orien X010). In some embodiments, the oncolytic virus comprises JX-593, Coxsackievirus A21 (CVA21), marabá virus or its MG1 variant, DNX2440 adenovirus, fowl pox virus, or Sendai virus. In some embodiments, the tyrosine kinase inhibitor is administered systemically, and the oncolytic virus is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the oncolytic virus. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the oncolytic virus. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the oncolytic virus. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the oncolytic virus. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the oncolytic virus.

In some embodiments, there is provided a method of treating a cancer (e.g., a solid tumor, e.g., a hematological cancer, e.g., a late stage cancer) in an individual, comprising administering to the individual a tyrosine kinase inhibitor and a sound treatment (e.g., high intensity focused ultrasound (HIFU), e.g., low intensity focused ultrasound (LIPUS)), optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and a sound treatment (e.g., high intensity focused ultrasound (HIFU), e.g., low intensity focused ultrasound (LIPUS)), wherein the tyrosine kinase inhibitor and the sound treatment are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the tyrosine kinase inhibitor is administered systemically, and the method comprises administering the sound treatment at the site of the cancer to be treated. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the sound treatment. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the sound treatment. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the sound treatment. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the sound treatment. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the sound treatment.

In some embodiments, there is provided a method of treating a cancer (e.g., a solid tumor, e.g., a hematological cancer, e.g., a late stage cancer) in an individual, comprising administering to the individual a tyrosine kinase inhibitor and a magnetic therapy (e.g., pulsed magnetic field, e.g., static magnetic field), optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and a magnetic therapy (e.g., pulsed magnetic field, e.g., static magnetic field), wherein the tyrosine kinase inhibitor and the magnetic therapy are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the tyrosine kinase inhibitor is administered systemically, and the method comprises administering the magnetic treatment at the site of the cancer to be treated. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the magnetic therapy. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the magnetic therapy. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the magnetic therapy. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the magnetic therapy. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the magnetic therapy.

In some embodiments, there is provided a method of treating a cancer (e.g., a solid tumor, e.g., a hematological cancer, e.g., a late stage cancer) in an individual, comprising administering to the individual a tyrosine kinase inhibitor and an electrical treatment or electrochemical treatment, optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and an electrical or electrochemical treatment, wherein the tyrosine kinase inhibitor and the electrical treatment or electrochemical treatment are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the tyrosine kinase inhibitor is administered systemically, and the method comprises administering the electrical treatment or electrochemical treatment at the site of the cancer to be treated. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the electrical treatment or electrochemical treatment. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the electrical treatment or electrochemical treatment. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the electrical treatment or electrochemical treatment. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the electrical treatment or electrochemical treatment. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the electrical treatment or electrochemical treatment.

In some embodiments, there is provided a method of treating a cancer (e.g., a solid tumor, e.g., a hematological cancer, e.g., a late stage cancer) in an individual, comprising administering to the individual a tyrosine kinase inhibitor and an electrostatic treatment, optionally wherein the tyrosine kinase inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a tyrosine kinase inhibitor and an electrostatic treatment, wherein the tyrosine kinase inhibitor and the an electrostatic treatment are administered within 24 hours (e.g., within 12, 8, 4, 2, or 1 hour, or within 30 minutes) of each other. In some embodiments, the tyrosine kinase inhibitor is administered intermittently. In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the tyrosine kinase inhibitor is administered systemically, and the method comprises administering the electrostatic treatment at the site of the cancer to be treated. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the electrostatic treatment. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the electrostatic treatment. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the electrostatic treatment. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the electrostatic treatment. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the electrostatic treatment.

In some embodiments, two or more proinflammatory agents described herein are administered to the individual. For example, in some embodiments, there is provided a method of treating a cancer (e.g., a solid tumor, e.g., a hematological cancer, e.g., a late stage cancer) in an individual, comprising administering a tyrosine kinase inhibitor, a TLR agonist or a STING activator (e.g., MSA-2, ADU-S100 or cGAMP), and an immune checkpoint inhibitor. In some embodiments, the TLR agonist activates one or more TLRs selected from the group consisting of TLR9, TLR4, TLR7 and TLR8. In some embodiments, the immune checkpoint inhibitor is an anti-PD-1 agent (such as an anti-PD-1 antibody), an anti-PD-L1 agent (such as an anti-PD-L1 antibody), or an anti-CTLA-4 agent (such as an anti-CTLA-4 antibody). In some embodiments, the tyrosine kinase inhibitor, the TLR agonist, and the immune checkpoint inhibitor are administered within the same day. In some embodiments, the tyrosine kinase inhibitor is administered intermittently. In some embodiments, the tyrosine kinase inhibitor, the TLR agonist, and/or the immune checkpoint inhibitor are administered at least twice (e.g., at least three, four, five or six times). In some embodiments, the tyrosine kinase inhibitor, the TLR agonist, and the immune checkpoint inhibitor are administered at least two cycles (e.g., at least three cycles), optionally administered within the same day for at least two consecutive days (e.g., at least three consecutive days) in each cycle. In some embodiments, each cycle has about seven to about twenty days. In some embodiments, the TLR agonist activates a TLR on a macrophage, optionally wherein the TLR comprises TLR9. In some embodiments, the TLR agonist activates at least two TLRs (e.g., TLR4, TLR7, TLR8, or TLR9). In some embodiments, the TLR agonist activates at least three TLRs (e.g., TLR9, TLR4 and TLR7/8). In some embodiments, the TLR agonist comprises CpG, polyI:C and/or R848. In some embodiments, the TLR agonist is selected from the group consisting of LTA, CpG, PolyI;C, LPS, MPLA, Flagellin, R848, Vesatolimod, Bropirimine, Motolimod, and Loxoribine. In some embodiments, the TLR agonist comprises CpG, polyI:C and R848, for example at 1:1:1 ratio. In some embodiments, the tyrosine kinase inhibitor is administered systemically, and the TLR agonist is administered intratumorally. In some embodiments, the tyrosine kinase inhibitor is administered systemically and intratumorally. In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the TLR agonist and/or the immune checkpoint inhibitor. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the TLR agonist and/or the immune checkpoint inhibitor. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the TLR agonist and/or the immune checkpoint inhibitor. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the TLR agonist and/or the immune checkpoint inhibitor. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the TLR agonist and/or the immune checkpoint inhibitor. In some embodiments, the TLR agonist is selected from the group consisting of LTA, CpG, PolyI;C, LPS, MPLA, Flagellin, R848, Vesatolimod, Bropirimine, Motolimod, and Loxoribine.

In some embodiments, there is provided a method of treating a cancer (e.g., a solid tumor, e.g., a hematological cancer, e.g., a cancer that is resistant or refractory to a checkpoint inhibitor, e.g., a late stage cancer) in an individual, comprising administering to the individual a tyrosine kinase inhibitor (e.g., a Src family kinase inhibitor such as any exemplified in table 2), a SHP-1 inhibitor (e.g., TPI-1 or an analog or a derivative thereof), and a TLR agonist or STING activator as discussed herein. In some embodiments, the TLR agonist is selected from the group consisting of LTA, CpG, PolyI;C, LPS, MPLA, Flagellin, R848, Vesatolimod, Bropirimine, Motolimod, and Loxoribine. In some embodiments, the method further comprises administering to the individual an agent that reduces systemic inflammation and/or reduces inflammatory cytokine cascade or cytokine storm (e.g., an anti-TNFα antibody or an anti-IL-6 antibody).

In some embodiments, there is provided a method of treating a cancer (e.g., a solid tumor, e.g., a hematological cancer, e.g., a late stage cancer) in an individual, comprising administering to the individual a tyrosine kinase inhibitor, wherein the individual is selected for treatment based upon the individual having an ongoing inflammation reaction. In some embodiments, the individual has an acute inflammation reaction. In some embodiments, the inflammation reaction is in the tumor. In some embodiments, the inflammation reaction is at a site distinct from the tumor. In some embodiments, the individual has an inflammation reaction when an inflammation reaction where there are at least two (e.g., two, three, four or five) events selected from the group consisting of a) an increase in one or more (e.g., at least one, two, three, four, five) inflammatory cytokines (such as IFNγ, IL-12b, TNFα, IL-6, IL-1β, IFN-α1, IFN-α2, IFN-β1), b) a decrease in one or more (e.g., at least one, two or three) anti-inflammatory cytokine (such as TGFβ1, TGFβ2, TGFβ3), c) an increase in the infiltrating immune cells (such as T cells, NK cells, macrophages, neutrophils), d) a decrease in suppressive immune cells (such as MDSCs), and/or e) an increase in one or more (e.g., at least one, two, three, four, or five) immunogenic co-stimulatory molecules (such as CD80, CD86, OX40L, CD40, ICOS-L, PD-L1, GITRL) in the tissue (e.g., tumor tissue) or immune cells (such as macrophages). In some embodiments, the tyrosine kinase inhibitor is administered intermittently. In some embodiments, the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets tyrosine kinase or activated tyrosine kinase). In some embodiments, the tyrosine kinase inhibitor is administered at least twice (e.g., at least three, four, five or six times). In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor.

In some embodiments, there is provided a method of treating a cancer (e.g., a solid tumor, e.g., a hematological cancer, e.g., a late stage cancer) in an individual, comprising administering to the individual a tyrosine kinase inhibitor, wherein the individual is selected for treatment based upon the individual having an ongoing immunogenic cell death (ICD). In some embodiments, the individual has ICD when a sample from the cancer has a higher level of one or more (e.g., at least 10%, 20%, 30%, 40%, 50%, 60%, 70%, 80%, 90% more) DAMPs than a reference sample (e.g., a corresponding sample in a healthy control, e.g., a sample from the cancer prior to the administration of a therapy that induces ICD. In some embodiments, the tyrosine kinase inhibitor is administered intermittently. In some embodiments, the DAMPs are selected from the group consisting of endoplasmic reticulum (ER) chaperones (e.g., calreticulin (CALR), e.g., heat-shock proteins (HSPs)), the non-histone chromatin-binding protein high-mobility group box 1 (HMGB1), the cytoplasmic protein annexin A1 (ANXA1), and the small metabolite ATP, and type I interferons (IFNs). In some embodiments, the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets tyrosine kinase or activated tyrosine kinase). In some embodiments, the tyrosine kinase inhibitor is administered at least twice (e.g., at least three, four, five or six times). In some embodiments, the method comprises administering the tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the tyrosine kinase inhibitor is administered systemically (e.g., intravenously, e.g., subcutaneously) and/or locally (e.g., intratumorally). In some embodiments, the tyrosine kinase inhibitor specifically inhibits SHP-1 signaling. In some embodiments, the tyrosine kinase inhibitor inhibits a Src family kinase (SFK). In some embodiments, the tyrosine kinase inhibitor is an Hck inhibitor. In some embodiments, the tyrosine kinase inhibitor inhibits any one or more of SRC, BLK, HCK, FYN, FGR and YES. In some embodiments, the tyrosine kinase inhibitor is selected from the group consisting of RK-20449, Dasatinib, and R406. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or a pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or a pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or a pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or a pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or a pro-inflammatory agent.

In some embodiments, the present application provides a method of treating a cancer (e.g., a solid tumor, e.g., a hematological cancer, e.g., a late stage cancer) in an individual, comprising administering to the individual a) monocytes or macrophages deficient in tyrosine kinase expression or activation and b) a pro-inflammatory agent (e.g., a TLR agonist, e.g., a radiation therapy). In some embodiments, the monocytes or macrophages are derived from the same individual. In some embodiments, the monocytes or macrophages are engineered to express a chimeric receptor targeting a tumor antigen. In some embodiments, the monocytes or macrophages and the pro-inflammatory agent are administered within 24 hours (e.g., within 12, 8, 4, 2, or 1 hour, or within 30 minutes) of each other. In some embodiments, the monocytes or macrophages and the pro-inflammatory agent are administered simultaneously, concurrently, or sequentially. In some embodiments, the monocytes or macrophages are administered prior to the pro-inflammatory agent. In some embodiments, the monocytes or macrophages are administered following the pro-inflammatory agent. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the monocytes or macrophages and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the monocytes or macrophages and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the monocytes or macrophages and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the monocytes or macrophages and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the monocytes or macrophages and/or the pro-inflammatory agent. In some embodiments, the TLR agonist is selected from the group consisting of LTA, CpG, PolyI;C, LPS, MPLA, Flagellin, R848, Vesatolimod, Bropirimine, Motolimod, and Loxoribine.

The present application also provides a method of modulating monocytes or macrophages derived from an individual having a cancer, comprising contacting the monocytes or macrophages with a tyrosine kinase inhibitor as described above, and a pro-inflammatory agent as described above. In some embodiments, the monocytes or macrophages are derived from the same individual. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the pro-inflammatory agent.

The present application also provides methods of activating phagocytosis against tumor cells in an individual having a tumor, comprising administering to the individual a tyrosine kinase inhibitor, wherein the individual a) has been subject to, is being subject to, or is about to be subject to a pro-inflammatory agent, or b) is under an inflammation reaction or has an ongoing infection. In some embodiments, the tyrosine kinase inhibitor is administered systemically (e.g., intravenously or subcutaneously). The present application also provides a method of activating tumor infiltrating T cells in an individual having a tumor comprising administering to the individual a tyrosine kinase inhibitor, wherein the individual a) has been subject to, is being subject to, or is about to be subject to a pro-inflammatory agent, or b) is under an inflammation reaction or has an ongoing infection. In some embodiments, the method comprises administering tyrosine kinase inhibitor to the individual at an interval of no more than once every three days for at least twice. In some embodiments, the method comprises administering the tyrosine kinase inhibitor to the individual for at least two cycles, wherein the tyrosine kinase inhibitor is administered for at least once in each cycle and wherein each cycle has about three to about twenty days. In some embodiments, the pro-inflammatory agent and the tyrosine kinase inhibitor are administered within 24 hours of each other. In some embodiments, the pro-inflammatory agent comprises an agent selected from the group consisting of a TLR agonist, a STING activator, a radiation therapy, a PAMP/DAMP activator, a checkpoint inhibitor, a pro-inflammatory cytokine, a chemotherapeutic agent, a bacteria component, a cancer vaccine, and an oncolytic virus. In some embodiments, the method further comprises administering (e.g., locally or systemically) to the individual an effective amount of a SHP-1 inhibitor (e.g., TPI-1 or an analog or a derivative thereof). In some embodiments, the method further comprises administering to the individual an agent that reduces systemic inflammation and/or reduces inflammatory cytokine cascade or cytokine storm (e.g., an anti-TNFα antibody or an anti-IL-6 antibody). In some embodiments, the agent that reduces systemic inflammation and/or reduces inflammatory cytokine cascade or cytokine storm (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered simultaneously with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered concurrently with the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered sequentially to (e.g., before or after) the tyrosine kinase inhibitor and/or the pro-inflammatory agent. In some embodiments, the agent that reduces systemic inflammation and/or reduces inflammatory cytokine cascade or cytokine storm (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor and/or the pro-inflammatory agent. In some embodiments, the TLR agonist is selected from the group consisting of LTA, CpG, PolyI;C, LPS, MPLA, Flagellin, R848, Vesatolimod, Bropirimine, Motolimod, and Loxoribine.

The cancer treatment methods described above can also be useful for 1) activating SHP-1 signaling pathway in an individual; 2) depleting the tyrosine kinase-iR-SHP-1 axis immunosuppression in the individual; 3) activating intratumoral anti-cancer innate and/or adaptive immunity in the individual; 4) unleashing TLR-induced proinflammatory response; and 5) increasing antigen presentation by tumor-relevance macrophages (TAM). The present application thus also provides methods for any one or more of these purposes.

### Tumor Microenvironment (TME) Immunosuppression and SHP-1 Signaling

Src homology region 2 (SH-2) domain-containing phosphatase 1 (SHP-1) is a non-receptor tyrosine phosphatase encoded by the PTPN6 gene that is located on human chromosome 12p13 and contains two promoter regions (within exon 1 and 2), giving rise to two forms of SHP-1 which differ in their N-terminal amino acid sequences but have a similar phosphatase activity. Promoter I is active in non-hematopoietic cells, while promoter II in hematopoietic-derived cells; in some epithelial cancer cells both promoters may function and generate various SHP-1-alternative transcripts. The two SHP-1 isoforms show different subcellular localizations: form I is mainly located in the nucleus, while form II is in the cytoplasm, suggesting that they have different targets.

SHP-1 is a 595 amino acid protein composed of two tandem N-terminal SH2 domains (N—SH2 and C—SH2), a classic catalytic protein tyrosine phosphatase (PTP) domain, and a C-terminal tail containing several phosphorylation sites. Its crystal revealed a structure in which the N—SH2 is bound to the catalytic site of the protein through charge-charge interaction. In this auto-inhibited inactive state the access of substrates to the active site is prevented, but binding of phosphotyrosine residues to the SH2 domains causes a conformational change that impairs the interaction between the N—SH2 and the catalytic domains. This opens the conformation to allow the access of substrate and is further stabilized by new interactions between SH2 domains and the catalytic domain. These molecular rearrangements determine a sophisticated regulatory mechanism controlled by substrate recruitment.

An additional mechanism of activation is mediated by the phosphorylation of amino acids within the C-terminal tail. So far, three phosphorylation sites have been found, two tyrosine (Tyr536 and Tyr564) and a serine (Ser591) residues. Tyr536 and Tyr564 become phosphorylated upon various stimuli (i.e., insulin stimulation or apoptosis inducers), giving rise to an increased SHP-1 activity. The molecular mechanism is not clear, although it has been proposed that Tyr phosphorylations could lead to interaction with the N—SH2 domain, releasing the inhibitory effect of this domain on the PTPase activity. SHP-1 activity can also be negatively regulated by protein kinase C (PKC) or mitogen-activated protein kinases (MAPKs) through phosphorylation at Ser591, whose mechanism of inhibition has not been well-characterized.

Protein-tyrosine phosphorylation is a reversible post-translational modification, tightly regulated by both kinases and phosphatases. Any deviation in the phosphorylation/dephosphorylation balance can promote the intracellular accumulation of tyrosine-phosphorylated proteins, which cause an altered regulation of cellular processes including cell growth, migration, invasion, differentiation, survival, and cellular trafficking. In this scenario, SHP-1 acts as a classical tumor suppressor, mainly involved in the homeostatic maintenance of potentially all these processes. SHP-1 function is indeed altered in both solid and hematological human cancers through somatic mutations or epigenetic mechanisms. Besides its well-documented role in the regulation of hematopoietic cell biology, SHP-1 has now been correlated to a number of signal transduction pathways relevant to cancer pathogenesis and progression.

However, inhibition of SHP-1 risks severe adverse effects. Studies of SHP-1 genetically deficient mice, the motheaten mice (me/me or me^{v}/me^{v}), reveal critical immunological abnormalities and hyperactivation of immune cells associated with the global loss of the tyrosine kinase. The motheaten mice usually succumb to life-threatening autoimmune inflammatory conditions at the early age. Even partial depletion of SHP-1 in WT mice after they grew to adults led to features of inflammatory disease, causing extensive lung inflammation and splenomegaly. Like a double-edged sword, inhibition of SHP-1, despite the potential of empowering anti-cancer immunity, inevitably endangers hosts for heightened inflammatory response, cytokine storm and autoimmunity.

Inhibitors targeting the tyrosine kinase phosphatase activity have been under development for some times, and some have now entered preclinical studies, including NSC-87877, sodium stibogluconate (SSG), tyrosine phosphatase inhibitor 1 (TPI-1 or an analog or a derivative thereof), and suramine; however, only a few of them have been shown to be active in experimental tumor models. SSG has been through Phase I trials for both malignant melanoma (NCT00498979) and advanced malignancies (NCT00629200); the drug was administrated in combination with interferons followed or not by chemotherapy treatment. Unfortunately, no effect was seen against tumor development, with the most common toxic side-effects being thrombocytopenia, elevated serum lipase, fatigue, fever, chills, anemia, hypokalemia, pancreatitis, and skin rash (observed in up to 68% of patients). At present, no SHP-1 inhibitor is under Phase II trial.

The SHP-1 inhibitors described herein can be administered along with the tyrosine kinase inhibitor. In some embodiments, the method comprises administering (e.g., locally or systemically) to the individual an effective amount of a SHP-1 inhibitor (e.g., TPI-1 or an analog or a derivative thereof). In some embodiments, the SHP-1 is administered simultaneously with the tyrosine kinase inhibitor. In some embodiments, the SHP-1 is administered sequentially (e.g., prior to or after) with the tyrosine kinase inhibitor. In some embodiments, the SHP-1 administration follows the same dosing schedule as the tyrosine kinase inhibitor.

An Agent that Reduces Systemic Inflammation

In some cases, individuals develop systemic inflammation, i.e., cytokine release syndrome (CRS) after receiving (e.g.) immunotherapeutic treatment, however the inflammatory disorder is not fully understood. CRS can be induced by direct target cell lysis and the consecutive release of cytokines like TNFα or IFNγ, or by activation of T cells due to therapeutic stimuli that is followed by subsequent cytokine release. These cytokines trigger a chain reaction due to the activation of innate immune cells like macrophages and endothelial cells, which then induces further cytokine release. In particular, IL-6, IL-10, and IFNγ are most commonly found to be elevated in patients with CRS.

The methods described herein can further comprises administration of an agent that reduces systemic inflammation (including, for example, an agent that reduces inflammatory cytokine cascade or cytokine storm, e.g., a TNFα inhibitor such as an anti-TNFα antibody), in order to curb down systemic inflammation and reduce adverse toxicity. These agents include, but are not limited to, inhibitors of TNFα, IL-6, IL-10, and IFNγ. In some embodiments, the agent that reduces systemic inflammation (e.g., a TNFα inhibitor, e.g., an anti-TNFα antibody) is administered prior to (e.g., within about any of one week, 7 days, 6 days, 5 days, 4 days, 3 days, 2 days, 1 day, or less prior to) the tyrosine kinase inhibitor. In some embodiments, the agent that reduces systemic inflammation (e.g., a TNFα inhibitor, e.g., an anti-TNFα antibody) is administered simultaneously with the tyrosine kinase inhibitor. In some embodiments, the agent that reduces systemic inflammation (e.g., a TNFα inhibitor, e.g., an anti-TNFα antibody) is administered concurrently with the tyrosine kinase inhibitor. In some embodiments, the agent that reduces systemic inflammation (e.g., a TNFα inhibitor, e.g., an anti-TNFα antibody) is administered sequentially (e.g., prior to or after) with the tyrosine kinase inhibitor. In some embodiments, the agent that reduces systemic inflammation (e.g., a TNFα inhibitor, e.g., an anti-TNFα antibody) is administered immediately after (e.g., within about any of 5 min, 10 min, 15 min, 30 min, 45 min, 60 min, 1 hr, 2 hrs, or 3 hrs after) the tyrosine kinase inhibitor. In some embodiments, the administration of the agent that reduces systemic inflammation (e.g., a TNFα inhibitor, e.g., an anti-TNFα antibody) follows the same dosing schedule as the tyrosine kinase inhibitor. In some embodiments, the agent that reduces systemic inflammation (e.g., a TNFα inhibitor, e.g., an anti-TNFα antibody) is administered at a sub-therapeutic dose, namely, at a dose that is lower than an effective amount for treating a disease when administered alone. In some embodiments, the administration of the agent that reduces systemic inflammation (e.g., a TNFα inhibitor, e.g., an anti-TNFα antibody) allows more frequent administration of the tyrosine kinase inhibitor and/or the proinflammatory agent (e.g., daily, once every two days, once every three days, etc.).

The agent can include any anti-inflammatory agent known in the art, including inhibitors of or antagonists to pro-inflammatory agents. For example, the agent can be an inhibitor or antagonist, including but not limited to, a small molecule inhibitor, a neutralizing antibody, a receptor blockade antibody, a soluble receptor, a targeting short interfering RNA (siRNA), a chemical inhibitor of mRNA stability, analogs or derivatives thereof, and any combination thereof, including combinations of agents targeting one or more molecules (e.g., targeting via the inhibition of TNFα alone, IL-6 alone, or TNFα and IL-6 in combination).

### Anti-TNFα Antagonist

TNFα, a major proinflammatory cytokine, is secreted by activated macrophages, monocytes and lymphocytes. Inventors surprisingly found that the administration of an anti-TNFα antibody to an individual who has been administered with a tyrosine kinase inhibitor and a proinflammatory agent alleviates toxicity caused by systemic inflammation without compromising the efficacy of the therapeutic agents.

The methods of the present application therefore in some embodiments comprises administration of a TNFα inhibitor, e.g., an anti-TNFα antagonist (e.g., in the context where the proinflammatory agent is not TNFα). In some embodiments, the TNFα inhibitor is selected from the group consisting of a small molecule inhibitor, a neutralizing antibody, a TNFα receptor blockade antibody, a soluble TNFα receptor, a TNFα-targeting short interfering RNA (siRNA), a chemical inhibitor of TNFα mRNA stability, an inhibitor of TNFα converting enzyme (TACE), and analogs or derivatives thereof. In some embodiments, the TNFα inhibitor is an anti-TNFα neutralizing antibody. In some embodiments, the TNFα inhibitor is an anti-TNFα receptor blockade antibody. In some embodiments, the anti-TNFα antibody is a monoclonal antibody. In some embodiments, anti-TNFα antibody is a chimeric, humanized and/or fully human antibody.

Suitable antibodies for use in the methods provided herein include, but are not limited to, Remicade® (Infliximab (Centocor)), and those antibodies described, for example, in U.S. Pat. Nos. 6,835,823; 6,790,444; 6,284,471; 6,277,969; 5,919,452; 5,698,195; 5,656,272; and 5,223,395 and in EP U.S. Pat. No. 610,201, the contents of each of which are hereby incorporated by reference in their entirety, or antibodies that bind to the same epitope as Remicade®. Others suitable anti-TNFα antibodies for use in the methods provided herein are, by way of non-limiting example, Humira (Adalimumab (Abbott Laboratories, Esai)) as described in U.S. Pat. Nos. 6,090,382; 6,258,562; or 6,509,015 and related patents and applications, the contents of which are hereby incorporated by reference in their entirety; Simponi™ (Golimimab, CNTO 148 (Centocor)) as described in PCT Publication No. WO 02/12502 and related patents and applications, the contents of which are hereby incorporated by reference in their entirety; ART621 (Arana Therapeutics), SSS 07 (Epitopmics and 3SBio) or antibodies that bind to the same epitope as Humira, Simponi, ART621 or SSS 07.

In some embodiments, the TNFα inhibitor, e.g., anti-TNFα antagonist, is a fusion protein. Suitable fusion proteins for use in the methods provided herein include, but are not limited to, Enbrel (Etanercept (Amgen)) and other fusion proteins or fragments thereof described in U.S. Pat. No. 5,712,155, PCT Publication No. WO 91/03553, and related patents and applications, the contents of which are hereby incorporated by reference in their entirety.

In some embodiments, the TNFα inhibitor, e.g., anti-TNFα antagonist, is a modified antibody antagonist or a non-antibody-based antagonist. Such antagonists include advanced antibody therapeutics, such as antibody fragments including, but not limited to, Cimzia™ (Certolizumab pegol, CDP870 (Enzon)), bispecific antibodies, Nanobodies® such as ABX 0402 (Ablynx), immunotoxins, and radiolabeled therapeutics; peptide therapeutics; gene therapies, particularly intrabodies; oligonucleotide therapeutics such as aptamer therapeutics, antisense therapeutics, interfering RNA therapeutics; and small molecules such as LMP-420 (LeukoMed) as described in EP U.S. Pat. No. 767,793, and related patents and applications, the contents of which are hereby incorporated by reference in their entirety.

In some embodiments, the TNFα inhibitor is administered systemically. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the individual does not develop cytokine release syndrome or pro-inflammatory organ damage. In some embodiments, administration of the TNFα inhibitor does not compromise or weakly compromises tumor clearance.

In some embodiments, the TNFα inhibitor is administered prior to (e.g., within about any of two weeks, one week, 7 days, 6 days, 5 days, 4 days, 3 days, 2 days, 1 day, or less prior to) the tyrosine kinase inhibitor and/or the proinflammatory agent. Exemplary TNFα inhibitors such an anti-TNFα antibody is usually stable for at least one or two weeks. In some embodiments, the TNFα inhibitor is administered simultaneously with the tyrosine kinase inhibitor and/or the proinflammatory agent. In some embodiments, the TNFα inhibitor is administered concurrently with the tyrosine kinase inhibitor and/or the proinflammatory agent. In some embodiments, the TNFα inhibitor is administered sequentially to (e.g., before or after) the tyrosine kinase inhibitor and/or the proinflammatory agent. In some embodiments, the TNFα inhibitor is administered immediately after (e.g., within about any of 5 min, 10 min, 15 min, 30 min, 45 min, 60 min, 1 hr, 2 hrs, or 3 hrs after) the tyrosine kinase inhibitor and/or the proinflammatory agent.

### Anti-IL6 Antagonist

An “anti-IL6 antagonist” or “IL6 inhibitor” refers to agent that inhibits or blocks IL6 biological activity via binding to IL6 or IL6 receptor. In some embodiments, the anti-IL6 antagonist is an antibody. In one embodiment, the anti-IL6 antagonist is an antibody that binds IL6 receptor. Antibodies that bind IL-6 receptor include tocilizumab (including intravenous, i.v., and subcutaneous, s.c., formulations thereof) (Chugai, Roche, Genentech), satralizumab (Chugai, Roche, Genentech), sarilumab (Sanofi, Regeneron), NI-1201 (Novimmune and Tiziana), and vobarilizumab (Ablynx). In one embodiment, the anti-IL6 antagonist is a monoclonal antibody that binds IL6. Antibodies that bind IL-6 include sirukumab (Centecor, Janssen), olokizumab (UCB), clazakizumab (BMS and Alder), siltuximab (Janssen), EBI-031 (Eleven Biotherapeutics and Roche). In one embodiment, the IL6 antagonist is olamkicept.

In some embodiments, the IL6 inhibitor is administered systemically. In some embodiments, the IL6 inhibitor is administered at least once a week, once every five days, once every three days, or daily. In some embodiments, the IL6 inhibitor is administered intermittently. In some embodiments, the IL6 inhibitor is administered to the individual for at least two cycles, wherein each cycle has about three to about seven days.

### Tyrosine Kinase Inhibitors

The tyrosine kinase inhibitors referred herein is an agent of any kind or sort that inhibits the expression or activation of tyrosine kinase.

In some embodiments, the tyrosine kinase inhibitor is capable of inhibiting at least about 20% (e.g., at least 20%, 30%, 40%, or 50%) of the tyrosine kinase activity. In some embodiments, the tyrosine kinase inhibitor is capable of inhibiting at least about 20% (e.g., at least 20%, 30%, 40%, or 50%) of the tyrosine kinase expression.

In some embodiments, the tyrosine kinase inhibitor specifically inhibits SHP-1 signaling.

In some embodiments, the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), a protein agent (e.g., an antibody agent that targets tyrosine kinase or activated tyrosine kinase, e.g., a dominant negative tyrosine kinase or a constitutively active tyrosine kinase mutant).

In some embodiments, the tyrosine kinase inhibitor has a half-life of no more than about 10, 9, 8, or 7 days (e.g., a half-life of no more than about 7, 6, 5, 4, 3, 2 or 1 day).

In some embodiments, the tyrosine kinase inhibitor is effective in inhibiting more than 50% of the tyrosine kinase activity for no more than about 10, 9, 8, 7, 6, or 5 days. In some embodiments, the tyrosine kinase inhibitor is effective in inhibiting more than 50% of the tyrosine kinase activity for no more than 4, 3, 2 or 1 day.

In some embodiments, the tyrosine kinase inhibitor is a covalent inhibitor. In some embodiments, the tyrosine kinase inhibitor is a noncovalent inhibitor.

In some embodiments, the tyrosine kinase inhibitor is a competitive inhibitor.

In some embodiments, the tyrosine kinase inhibitor is a nucleic acid editing system (such as a CRISPR system). In some embodiments, the CRISPR components are introduced into the cell (e.g., the monocytes and the macrophages) but no DNA encoding a guide RNA or Cas9 are incorporated into the cell's genome. Under this approach, the CRISPR system only cleave the cell's genomic DNA for a limited period of time. See e.g., Fister et al., Front Plant Sci. 2018 Mar. 2; 9:268.

In some embodiments, the tyrosine kinase inhibitor is administered at least two times (such as at least 3, 4, 5, or 6 times).

In some embodiments, the method comprises administering the tyrosine kinase inhibitor at an interval of no more than once every two days for at least twice (such as at least three times, four times, five times, or six times).

In some embodiments, the method comprises administering the tyrosine kinase inhibitor at an interval of no more than once every three days for at least twice (such as at least three times, four times, five times, or six times).

In some embodiments, the method comprises administering the tyrosine kinase inhibitor for at least two cycles. In some embodiments, the tyrosine kinase inhibitor is administered for at least once (e.g., for twice, three times, four times) in each cycle. In some embodiments, each cycle has about three to about 50 days (e.g., about 3-40 days, about 3-30 days, about 3-20 days, about 3-15 days, about 3-10 days, or about 2-10 days).

In some embodiments, the tyrosine kinase inhibitor is administered systemically (e.g., orally, intravenously, subcutaneously, intraperitoneally). In some embodiments, the tyrosine kinase inhibitor is administered locally (e.g., intratumorally). In some embodiments, the tyrosine kinase inhibitor is administered both systemically and locally (e.g., intratumorally).

In some embodiments, the tyrosine kinase inhibitor is complexed with a delivery vehicle before being administered into the individual. In some embodiments, the delivery vehicle promotes the delivery into the tumor.

In some embodiments, the tyrosine kinase inhibitor modulates a monocyte or macrophage (e.g., a monocyte or macrophage derived from the individual to be treated) in vitro.

In some embodiments, the tyrosine kinase inhibitor and the pro-inflammatory agent below are administered within 24 hours (e.g., within 12, 8, 4, 2, or 1 hour, or within 30 minutes) of each other. In some embodiments, the tyrosine kinase inhibitor and the pro-inflammatory agent are administered simultaneously, concurrently, or sequentially. In some embodiments, the tyrosine kinase inhibitor is administered prior to the pro-inflammatory agent. In some embodiments, the tyrosine kinase inhibitor is administered following the pro-inflammatory agent.

### Src Family Tyrosine Kinases (SFKs) and ITIMs Phosphorylation in TAM

In some embodiments, the tyrosine kinase is a tyrosine kinase of the Src family. Src-family kinases have a similar structure, comprised of an N-terminal Src-homology (“SH”) 4 (“SH4”) domain, a “unique” domain, an SH3 domain, an SH2 domain, a catalytic domain (also known as the SH1 domain or the kinase domain) and a short C-terminal tail. Activity is regulated by tyrosine phosphorylation at two sites. Phosphorylation of a tyrosine (Tyr-505, Src numbering) in the C-terminal tail leads to down-regulation by promoting an intramolecular interaction between the tail and the SH2 domain. The eight known mammalian members of the Src-family break down into two sub-families. Lck is most similar to Hck, Lyn and Blk (identities greater than 65% between any two members). The other sub-family consists of Src, Yes, Fyn and Fgr (identities greater than 70% between any two members). Residues that are important for Src-family kinase activity and/or substrate specificity have been identified by X-ray crystal structures and by structural modeling studies, and are highly conserved among family members.

The Src family of non-receptor tyrosine kinases (SFK) comprise of SRC, LCK, LYN, BLK, HCK, FYN, FGR and YES (8/9 members expressed in human), which can be divided into two groups according to their expression pattern. SRC, YES and FYN are ubiquitously expressed, while LCK, FGR, BLK, LYN, YRK and HCK show specific expression in certain types of cells and tissues.

In immune system, SFKs play important regulatory functions in both myeloid lineage and lymphoid lineage immune cells, controlling cell activation, proliferation, differentiation, apoptosis, cytokine production, migration, metabolism, etc.

LCK is specifically expressed in T cells and critically involved in TCR-mediated T cell activation; deficiency of LCK nullifies TCR signaling, hence diminishing antigen specific T cell activation, proliferation, and T cell immunity. LCK is not expressed in macrophages or other myeloid leukocytes.

LYN is highly expressed in B cells and is also expressed in myeloid leukocytes. In macrophages, our study found that LYN maintains constitutive activity and mediates low level tyrosine phosphorylation in the cytoplasmic ITIMs of iRs (inhibitory receptors). However, LYN appears not to be involved in stimuli induced, robust ITIMs tyrosine phosphorylation of iRs. Particularly, we found under tumor therapeutic conditions that HCK, or its related complementary SFKs (e.g. FGR and YES; See: Lowell C A, Soriano P, Varmus H E. Functional overlap in the src gene family: inactivation of hck and fgr impairs natural immunity. Genes and Development. 1994; 8:387-398.), phosphorylates ITIMs of iRs, leading to docking and activation of SHP-1, which mediates downstream inhibitory regulation.

FIG. 12 shows an example by studying SIRPα, an iR abundantly expressed in tumor-associated macrophages (TAM). As shown, proinflammatory stimuli (TLR agonists, proinflammatory cytokines IL-1β, IL-6, IL-12, IL-17, IL-18, TNFα, IFNγ, etc., and cancer therapies) induce SIRPα ITIMs phosphorylation and exclusive association of SHP-1 (not SHP-2). Anti-inflammatory cytokine stimulation also induces SIRPα ITIMs phosphorylation but association with SHP-2.

In the cases of macrophage activation by proinflammatory stimuli or cancer therapies, inhibition of Src family tyrosine kinases (SFK) by PP1 and PP2 (both SFK inhibitors), but not by inhibitors targeting other TKs, such as JAK (JAK inh.), Btk (LFMA-13) or Syk (Piceatannol), diminished SIRPα cytoplasmic ITIMs phosphorylation and SIRPα association with SHP-1. In comparison, the specific inhibitors towards LYN, bafetinib (also termed INNO-406), had only minor effect. Test macrophages with Lyn deficiency confirmed that Lyn has no effect on proinflammatory factor-induced SIRPa ITIMs phosphorylation and the association of SIRPα with SHP-1, despite that Lyn deficiency notably affected CD47 ligation induced low level SIRPα ITIMs phosphorylation in the absence of proinflammatory stimulation.

It is worthy to note that in the absence of therapies, immunosuppressive tumor TME are controlled by IL-10, TGFβ, and IL-4/13, which activate Bruton's tyrosine kinase (Btk) in macrophages (TAM), leading to phosphorylation of cytoplasmic ITIMs of iRs (e.g. SIRPα) and docking of SHP-2, but not SHP-1. This axis of events, where immunosuppressive cytokines activate Btk to drive SIRPα-SHP-2 binding, further enhances immunosuppressive signal transduction within TAM. As an additional consequence of this pathway, iRs expression on TAMs are further increased, thus serving as a feed-forward mechanism that controls TAM, and as such, the TME immunosuppression.

In some embodiments, the TKi inhibits a Src family kinase (SFK), optionally wherein the SFK is selected from SRC, LCK, LYN, BLK, HCK, FYN, FGR and YES. In some embodiments, the TKi inhibits a SFK that is not LYN. In some embodiments, the TKi inhibits a SFK that is not HCK. In some embodiments, SFK is selected from the group consisting of SRC, BLK, HCK, FYN, FGR and YES. In some embodiments, the SFK is HCK or its related complementary SFKs (e.g. FGR and YES). In some embodiments, the SFK is selected from the group consisting of HCK, FGR and YES.

In some embodiments, the tyrosine kinase inhibitor is a Src inhibitor. In some embodiments, the tyrosine kinase inhibitor is a Syk inhibitor. In some embodiments, the tyrosine kinase inhibitor is an Hck inhibitor. In some embodiments, the tyrosine kinase inhibitor inhibits any one or more (such as any of 2, 3, 4, 5, or 6) of: Src, Syk, Hck, Lck, Lyn, and Yes. In some embodiments, the tyrosine kinase inhibitor inhibits Bcr-Abl. In some embodiments, the tyrosine kinase inhibitor is selected from the group consisting of RK-20449, Dasatinib, and R406. In some embodiments, the tyrosine kinase inhibitor is selected from the group consisting of Ponatinib, Bosutinib, Saracatinib and KX2-391. These tyrosine kinase inhibitors are further discussed below.

### Src Inhibitors

Src is a member of non-receptor protein tyrosine kinases, and has an activity that phosphorylates a specific tyrosine residue in a target protein. The Src may be originated any species of animals (e.g., mammals), and for example may be at least one selected from the group consisting of primate Src including human Src (e.g., Accession No. NP_005408), monkey Src (e.g., Accession No. XP_002830325), and the like, and rodent Src including mouse Src (e.g., Accession No. NP_001020566), rat Src (e.g., Accession No. NP_114183), and the like, but not be limited thereto.

In some embodiments, the Src inhibitor (SRCi) may be an inhibitor of Src gene or Src protein expression; or an inhibitor of Src protein activity. The Src gene or Src protein expression inhibitor may be one or more selected from the group consisting of antisense nucleotides complementarily binding to mRNA of the gene, short interfering RNA (siRNA), short hairpin RNA (shRNA) and ribozyme, but not limited thereto. Further, the Src protein activity inhibitor may be one or more selected from the group consisting of a compound, a peptide, peptide mimetics, aptamers, antibodies, and natural products that specifically bind to the protein, but not limited thereto. The antibody includes a monoclonal antibody, a polyclonal antibody, or a recombinant antibody capable of specifically binding to the Src protein, and can be constructed by known methods known to those skilled in the art or purchased and used. According to the present disclosure, the compound may be one or more selected from the group consisting of dasatinib, bosutinib, ponatinib, saracatinib, WH-4-023, KX2-391, and WZ3105.

In one embodiment, the Src inhibitor may be at least one selected from the group consisting of dasatinib, saracatinib, and bosutinib, or any combination thereof.

KX2-391 (Tirbanibulin), which is also called N-benzyl-2-(5-(4-(2-morpholinoethoxy)phenyl)pyridin-2-yl)acetamide, has the following structure:

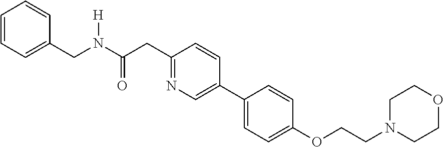 [SMILES: O=C(Cc1ccc(-c2ccc(OCCN3CCOCC3)cc2)cn1)NCc1ccccc1]

Dasatinib, which is also called N-(2-chloro-6-methylphenyl)-2-[[6-[4-(2-hydroxyethyl)-1-piperazinyl]-2-methyl-4-pyrimidinyl]amino]-5-thiazole carboxamide monohydrate, has the following structure:

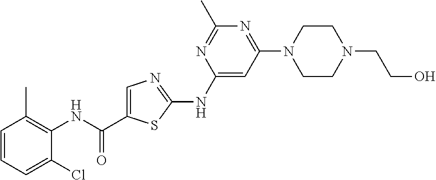 [SMILES: Cc1nc(Nc2ncc(C(=O)Nc3c(C)cccc3Cl)s2)cc(N2CCN(CCO)CC2)n1]

Saracatinib, which is also called AZD0530 (4-Quinazolinamine, N-(5-Chloro-1,3-benzodioxol-4-yl)-7-[2-(4-methyl-1-piperazinyl)ethoxy]-5-[(tetrahydro-2H-pyran-4-yl)oxy]-4-quinazolinamine), has the following structure:

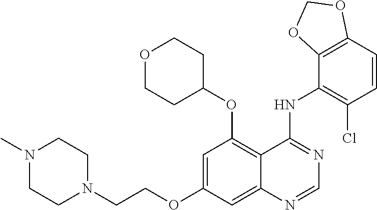 [SMILES: CN1CCN(CCOc2cc(OC3CCOCC3)c3c(Nc4c(Cl)ccc5c4OCO5)ncnc3c2)CC1]

Bosutinib, which is also called 4-[(2,4-dichloro-5-methoxyphenyl)amino]-6-methoxy-7-[3-(4-methylpiperazin-1-yl)propoxy]quinoline-3-carbonitrile, has the following structure:

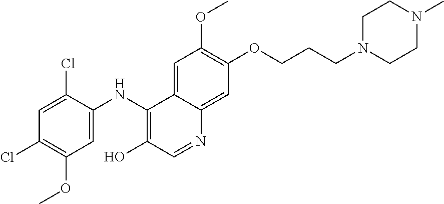 [SMILES: COc1cc(Nc2c(O)cnc3cc(OCCCN4CCN(C)CC4)c(OC)cc23)c(Cl)cc1Cl]

### Syk Inhibitors

Spleen tyrosine kinase (Syk) is a cytosolic non-receptor protein tyrosine kinase (PTK). The human SYK gene is located in the region of chromosome 9 q22. Syk, along with ZAP70, is a member of the Syk family of tyrosine kinases. These cytoplasmic non-receptor tyrosine kinases share a characteristic dual SH2 domain separated by a linker domain.

In some embodiments, the Syk inhibitor may be an inhibitor of Syk gene or Syk protein expression; or an inhibitor of Syk protein activity. The Syk gene or Syk protein expression inhibitor may be one or more selected from the group consisting of antisense nucleotides complementarily binding to mRNA of the gene, short interfering RNA (siRNA), short hairpin RNA (shRNA) and ribozyme, but not limited thereto. Further, the Syk protein activity inhibitor may be one or more selected from the group consisting of a compound, a peptide, peptide mimetics, aptamers, antibodies, and natural products that specifically bind to the protein, but not limited thereto. The antibody includes a monoclonal antibody, a polyclonal antibody, or a recombinant antibody capable of specifically binding to the Syk protein, and can be constructed by known methods known to those skilled in the art or purchased and used.

In some embodiments, the Syk inhibitor is a small molecule inhibitor. In some embodiments, the Syk inhibitor is selected from the group consisting of Entospletinib (GS-9973), Fostamatinib (R788), R406, Cerdulatinib (PRT0626070), and TAK-659.

In some embodiments, the Syk inhibitor is R406 having the formula as follows:

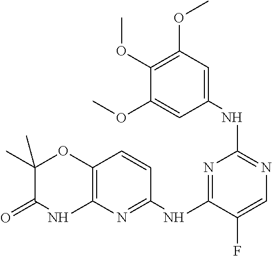 [SMILES: COc1cc(Nc2ncc(F)c(Nc3ccc4c(n3)NC(=O)C(C)(C)O4)n2)cc(OC)c1OC]

### Hck Inhibitors

Hck is a member of the Src-family of non-receptor tyrosine kinases, which plays many roles in signaling pathways involved in the regulation of cell processes. Hck is expressed in cells of hematopoietic origin, specifically myelomonocytic cells and B lymphocytes. It participates in phagocytosis, adhesion, migration, regulation of protrusion formation on cell membrane, lysosome exocytosis, podosome formation and actin polymerization. High levels of Hck are present in chronic myeloid leukemia and other hematologic tumors. Hck could also play a role in the genesis of acute myeloid leukemia.

In some embodiments, the Hck inhibitor may be an inhibitor of Hck gene or Hck protein expression; or an inhibitor of Hck protein activity. The Hck gene or Hck protein expression inhibitor may be one or more selected from the group consisting of antisense nucleotides complementarily binding to mRNA of the gene, short interfering RNA (siRNA), short hairpin RNA (shRNA) and ribozyme, but not limited thereto. Further, the Hck protein activity inhibitor may be one or more selected from the group consisting of a compound, a peptide, peptide mimetics, aptamers, antibodies, and natural products that specifically bind to the protein, but not limited thereto. The antibody includes a monoclonal antibody, a polyclonal antibody, or a recombinant antibody capable of specifically binding to the Hck protein, and can be constructed by known methods known to those skilled in the art or purchased and used.

In some embodiments, the Hck inhibitor is a small molecule inhibitor. In some embodiments, the Hck inhibitor is selected from the group consisting of RK-20449, RK-20693, RK-24466, RK-20444, RK-20445, and RK-20466. In other embodiments, the HCK inhibitor is selected from RK-20449, RK-20693, RK-24466, RK-20444, RK-20445, RK-20466, RK-20730, RK-20690, RK-20781, RK-20786, RK-20888, RK-20658, RK-20686, RK-20696, RK-20709, RK-20721, RK-20694, RK-20703, RK-20718, RK-20744, and compounds having Hck inhibitory activity disclosed in WO2014/017659, incorporated herein by reference. Hck inhibitors are also disclosed in WO2018/052120, which are incorporated herein by reference.

RK-20449 (also known as A 419259): 7-((1R,4R)-4-(4-methylpiperazin-1-yl)cyclohexyl)-5-(4-phenoxyphenyl)-7H-pyrrolo[2,3-d]pyrimidin-4-amine has a structure as follows:

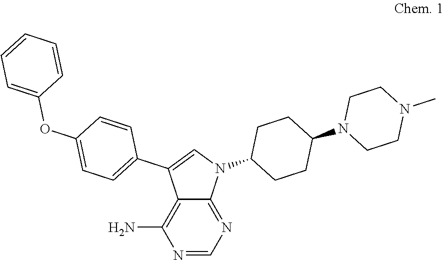 [SMILES: CN1CCN([C@H]2CC[C@H](n3cc(-c4ccc(Oc5ccccc5)cc4)c4c(N)ncnc43)CC2)CC1]

### Lck Inhibitors

Lck (or lymphocyte-specific protein tyrosine kinase) is a member of Src kinase family important for the activation of the T-cell receptor signaling in both naive T cells and effector T cells. The N-terminal tail of Lck is myristoylated and palmitoylated, which tethers the protein to the plasma membrane of the cell. The protein furthermore contains a SH3 domain, a SH2 domain and in the C-terminal part the tyrosine kinase domain.

In some embodiments, the Lck inhibitor may be an inhibitor of Lck gene or Lck protein expression; or an inhibitor of Lck protein activity. The Lck gene or Lck protein expression inhibitor may be one or more selected from the group consisting of antisense nucleotides complementarily binding to mRNA of the gene, short interfering RNA (siRNA), short hairpin RNA (shRNA) and ribozyme, but not limited thereto. Further, the Lck protein activity inhibitor may be one or more selected from the group consisting of a compound, a peptide, peptide mimetics, aptamers, antibodies, and natural products that specifically bind to the protein, but not limited thereto. The antibody includes a monoclonal antibody, a polyclonal antibody, or a recombinant antibody capable of specifically binding to the Lck protein, and can be constructed by known methods known to those skilled in the art or purchased and used.

In some embodiments, the Lck inhibitor is a small molecule inhibitor. In some embodiments, the Lck inhibitor is selected from the group consisting of Saractinib, Masitinib, and NVP-BEP800.

### Bcr-Abl Inhibitor

BCR-ABL, a fusion gene created as a consequence of a reciprocal translocation mutation in the long arms of Chromosome 9 and 12, encodes the BCR-ABL protein, a constitutively active cytoplasmic tyrosine kinase present in >90% of all patients with chronic myelogenous leukemia (CML) and in 15-30% of adult patients with acute lymphoblastic leukemia (ALL). Exemplary Bcr-Abl inhibitors include, but are not limited to, imatinib, nilotinib, dasatinib, bosutinib, ponatinib, bafetinib, rebastinib, tozasertib, danusertib, HG-7-85-01, GNF-2, and 1,3,4-thiadiazole analogs or derivatives. Additional Bcr-Abl inhibitors can be found, for example, at WO2006/052810, specifically incorporated herein by reference.

Ponatinib (AP24534) is a dual Src/Abl inhibitor having the following structure.

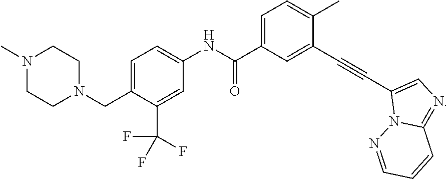 [SMILES: Cc1ccc(C(=O)Nc2ccc(CN3CCN(C)CC3)c(C(F)(F)F)c2)cc1C#Cc1cnc2cccnn12]

### Pro-Inflammatory Agents

Infection and tissue injury are the two classic instigators of inflammation. See e.g., Medzhitov, Nature. 2008 Jul. 24; 454 (7203): 428-35. Pro-inflammatory agents described herein include at least two overlapping categories: 1) an agent or therapy of any kind or sort that can promote an inflammation (e.g., by promoting one or more pro-inflammatory cytokines or chemokines, inhibiting one or more anti-inflammatory cytokines or chemokines, recruiting macrophages, NK cells, neutrophils, effector T cells, or B cells to the tissue or activating any of these cells, or suppressing regulatory/suppressive immune cells such as regulatory T cells or MDSC), and 2) an agent or therapy that can cause damage of cancer cells (e.g., necrosis of cancer cells).

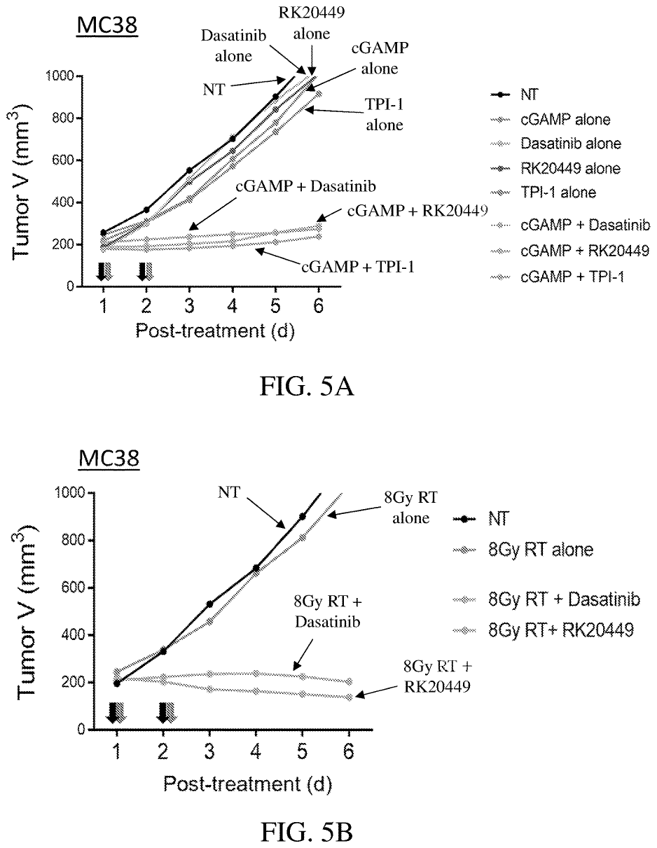

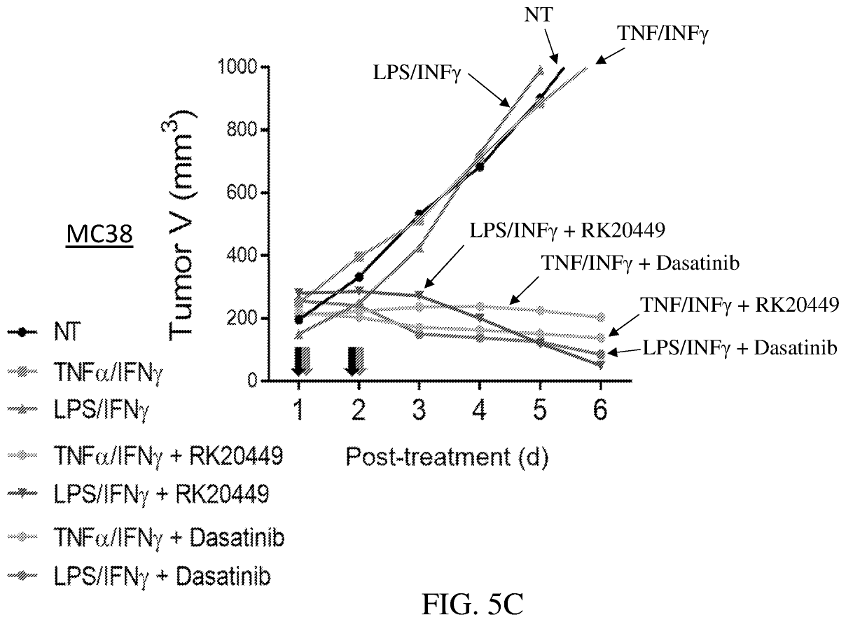

In some embodiments, the pro-inflammatory agent triggers a pro-inflammatory signal on macrophages. See e.g., FIG. 5A. In some embodiments, the pro-inflammatory agent activates a TLR, a TNFR, or ITAM-R. See Lionel et al., Eur J Immunol. 2011 September; 41 (9): 2477-2481. The pro-inflammatory can activate a pro-inflammatory signal on macrophages via a direct manner or indirect manner. For example, a TLR agonist, which directly activates TLR on macrophages, or a radiotherapy which indirectly activates a pro-inflammatory signal on macrophages, when used with a tyrosine kinase inhibitor both demonstrated remarkable anti-tumor effects. See the Examples.

Exemplary pro-inflammatory agents include TLR agonists, STING activators, radiation therapies, PAMP/DAMP activators, checkpoint inhibitors, pro-inflammatory cytokines or chemokines, chemotherapies, bacteria components, cancer vaccines, and oncolytic viruses. Other exemplary pro-inflammatory agents include sound treatments (e.g., high intensity focused ultrasound), magnetic therapies, electrical treatments, and electrostatic treatments that can kill cancer cells. See e.g., Naud et al., Nanoscale Adv., 2020, 2, 3632-3655; Rominiyi et al., Br J Cancer. 2021 February; 124 (4): 697-709; Zandi et al., Cancer Med. 2021 November; 10 (21): 7475-7491.

In some embodiments, the pro-inflammatory agent comprises an agent selected from the group consisting of TLR agonists, STING activators, radiation therapies, PAMP/DAMP activators, checkpoint inhibitors, pro-inflammatory cytokines or chemokines, chemotherapies, bacteria components, cancer vaccines, oncolytic viruses, sound treatments (e.g., high intensity focused ultrasound), magnetic therapies, electrical treatments, and electrostatic treatments.

In some embodiments, the pro-inflammatory agent comprises an agent selected from the group consisting of TLR agonists, STING activators, PAMP/DAMP activators, pro-inflammatory cytokines or chemokines, bacteria components, cancer vaccines, sound treatments (e.g., high intensity focused ultrasound), magnetic therapies, electrical treatments, and an electrostatic treatment.

In some embodiments, the pro-inflammatory agent is a sound treatment (e.g., high intensity focused ultrasound (HIFU), e.g., low intensity focused ultrasound (LIPUS)). See e.g., Wood et al., Ultrasound Med Biol. 2015 April; 41 (4): 905-928; Sengupta et al., J Adv Res. 2018 November; 14:97-111.

In some embodiments, the pro-inflammatory agent is a magnetic therapy (e.g., pulsed magnetic field, e.g., static magnetic field). See e.g., Tatarov et al., Comp Med. 2011 August; 61 (4): 339-345; Sengupta et al., J Adv Res. 2018 November; 14:97-111.

In some embodiments, the pro-inflammatory agent is an electrical treatment or electrochemical treatment. See e.g., Ciria et al., Chin J Cancer Res. 2013 April; 25 (2): 223-234; Das et al., Front Bioeng Biotechnol. 2021; 9:795300.

In some embodiments, the pro-inflammatory agent is an electrostatic treatment. See e.g., Zandi et al., Cancer Med. 2021 November; 10 (21): 7475-7491.

In some embodiments, the pro-inflammatory agent is a thermoacoustic treatment. See e.g., Wen et al., Theranostics. 2017; 7 (7): 1976-1989.

In some embodiments, the pro-inflammatory agent comprises a microbe (e.g., a fragment or lysate of a microbe). Examples of microbe include bacteria, fungi, and viruses.

### TLR Agonists

In some embodiments, the pro-inflammatory agent comprises or is a TLR agonist.

TLRs play a vital role in activating immune responses. TLRs recognize conserved pathogen-associated molecular patterns (PAMPs) expressed on a wide array of microbes, as well as endogenous DAMPs released from stressed or dying cells. TLR1, -2, -4, -5, -6, and -10 are expressed on the cell surface, whereas TLR3, -7, -8, and -9 are situated on endosomal membranes within the cell. TLR1 and TLR2 can heterodimerize to recognize a variety of bacterial lipid structures and cell wall components, such as triacylated lipoproteins, lipoteichoic acid, and β-glucans. TLR2 also heterodimerizes with TLR6 to bind diacylated lipopeptides. Additionally, TLR2 can bind various endogenous DAMPs, such as HSPs, HMGB1, uric acid, fibronectin, and other extracellular matrix proteins. It has also been suggested that TLR1 and TLR6 can heterodimerize with TLR10; however, the TLR agonist recognized by this dimer remains to be identified. TLR3 recognizes viral dsRNA, as well as synthetic analogs of dsRNA, such as ligand Poly I:C. TLR4 binds LPS in complex with lipid A binding protein, CD14, and myeloid differentiation protein 2, MD2 as well as recognizing various DAMPs. Endogenous TLR4 ligands, which have been described, include β-defensin 2, fibronectin extra domain A EDA, HMGB1, Snapin, and tenascin C. TLR5 recognizes bacterial flagellin, TLR7 and TLR8 bind viral ssRNA, whereas TLR9 interacts with unmethylated CpG DNA from bacteria and some viruses. Additional TLRs have been identified more recently in mice based on sequence homology of the highly conserved TIR domain. TLR10 is a surface receptor whose natural ligand remains unknown. TLR11, -12, and -13 are present in mice but not in humans. TLR11 was shown to bind a T. gondii profilin and uropathogenic Escherichia coli. The ligand for TLR12 has not yet been identified, whereas TLR13 is an endosomal receptor that recognizes VSV. See e.g., Kaczanowska et al., J Leukoc Biol. 2013 June; 93 (6): 847-63.

TLR signaling can act as a double-edged sword in cancer. It was found that TLR stimulation of cancer cells can lead to either tumor progression or inhibition. For example, Stimulation of TLR 2, 4, and 7/8 was found to lead to tumor progression via production of immunosuppressive cytokines, increased cell proliferation and resistance to apoptosis. R848-stimulation of TLR7/8 overexpressing pancreatic cancer cell line resulted in increased cell proliferation and reduced chemosensitivity. On the other hand, stimulation of TLR 2, 3, 4, 5, 7/8, and 9, often combined with chemo- or immunotherapy, can lead to tumor inhibition via different pathways. See e.g., Grimmig et al., Int J Oncol. (2015) 47:857-66; Urban-Wojciuk et al., Front Immunol. 2019; 10:2388.

In some embodiments, the TLR agonist activates any of the TLRs.

In some embodiments, the TLR agonist activates TLR1 or TLR2, optionally wherein the TLR agonist comprises a triacylated lipoprotein, a peptidoglycan, zymosan, and/or Pam₃CSK₄.

In some embodiments, the TLR agonist activates any one of TLR2, TLR3, TLR4, TLR5, and TLR6, optionally wherein the TLR agonist comprises a diacylated lipopeptide, a hot shock protein, HMGB1, uric acid, fibronectin, and/or ECM protein.

In some embodiments, the TLR agonist activates TLR2, optionally wherein the TLR agonist comprises Pam3Cys, SMP-105, and/or CBLB612.

In some embodiments, the TLR agonist activates TLR3, optionally wherein the TLR agonist comprises dsRNA, Poly I:C, PolyICIC, Poly-IC12U, IPH302, ARNAX, and/or MPLA.

In some embodiments, the TLR agonist activates TLR4, optionally wherein the TLR agonist comprises LPS, lipoteichoic acid beta-defensin 2, fibronectin EDA, HMGB1, snapin, tenascin C, OK-432, AS04, and/or GLA-SE.

In some embodiments, the TLR agonist activates TLR5, optionally wherein the TLR agonist comprises flagellin, CBLB502, and/or M-VM3.

In some embodiments, the TLR agonist activates TLR6.

In some embodiments, the TLR agonist activates TLR7 or TLR8, optionally wherein the TLR agonist comprises ssRNA, CpG-A, poly G10, and/or poly G3.

In some embodiments, the TLR agonist activates TLR7, optionally wherein the TLR agonist comprises bistriazolyl and/or R848.

In some embodiments, the TLR agonist activates TLR8, optionally wherein the TLR agonist comprises VTX1463 and/or R848.

In some embodiments, the TLR agonist activates TLR9, optionally wherein the TLR agonist comprises unmethylated CpG DNA, CpG (e.g., CpG-7909, KSK-CpG, CpG-1826), MGN1703, dsSLIM, IMO2055, SD101, and/or ODN M362.

In some embodiments, the TLR agonist activates TLR10, optionally wherein the TLR agonist comprises Pam₃CSK₄.

In some embodiments, the TLR agonist activates TLR11, optionally wherein the TLR agonist comprises Toxoplasma gondii profilin.

In some embodiments, the TLR agonist activates TLR12.

In some embodiments, the TLR agonist activates TLR13, optionally wherein the TLR agonist comprises VSV.

In some embodiments, the TLR agonist activates a TLR on a macrophage.

In some embodiments, the TLR agonist activates TLR1, TLR2, TLR3, TLR4, TLR7, TLR8, and/or TLR9.

In some embodiments, the TLR comprises TLR1, TLR4, and/or TLR9. In some embodiments, the TLR comprises TLR9.

In some embodiments, the TLR comprises TLR2, TLR4, TLR7, and/or TLR8.

In some embodiments, the TLR agonist comprises CpG. In some embodiments, the TLR agonist comprises polyI:C. In some embodiments, the TLR agonist comprises CpG and/or polyI:C. In some embodiments, the TLR agonist comprises CpG, polyI:C and/or R848. In some embodiments, the TLR agonist comprises CpG, polyI:C and R848, for example at 1:1:1 ratio.

In some embodiments, the method described herein further comprises assessing whether the individual has an ongoing infection. In some embodiments, a reduced amount of the TLR agonist is administered when the individual has an ongoing infection. In some embodiments, the administration of TLR agonist can be avoided when the individual has an ongoing infection.

### Radiation Therapy

In some embodiments, the pro-inflammatory agent comprises or is a radiation therapy. Radiation activates the interconnected network of cytokines, adhesion molecule, ROS/RNS and DAMPs leading to a self-amplified cascade, which generates pro-inflammatory, pro-oxidant tumor microenvironment and ultimately tumor cell death. See e.g., McKelvey et al., Mamm Genome. 2018; 29 (11): 843-865.

In some embodiments, the radiation therapy comprises irradiation at site of the cancer to be treated.

In some embodiments, the radiation therapy comprises irradiation at a site that is different from the site of the cancer to be treated.

In some embodiments, the radiation therapy is intraoperative radiation therapy (“IORT”). In particular embodiments, the radiation is localized to a tumor site. The patient may be subjected to intraoperative radiation prior to resection of the tumor or following resection of the tumor. The tumor site may comprise different types of cells including cancerous and benign cells. In certain embodiments, the radiation therapy is stereotactic body radiotherapy (“SBRT”) or stereotactic radiosurgery (“SRS”).

In some embodiments, the radiation is ionizing radiation such as particle beam radiation. The particle beam radiation may be selected from any of electrons, protons, neutrons, heavy ions such as carbon ions, or pions. The ionizing radiation may be selected from x-rays, UV-light, Y-rays, or microwaves. In some embodiments, the radiation therapy may comprise subjecting the patient to one or more types of radiation therapy.

In some embodiments, a radio sensitizer is used to sensitize the tumor cells to radiation. The use of such pharmaceuticals, called radiosensitizers, provides a method of increasing the radiosensitivity of tumors to radiation therapy, avoiding the need to increase radiation dosages to levels that are harmful to surrounding organs and tissues. See e.g., U.S. Pat. No. 9,656,098B2.

In some embodiments, the dose of the radiation therapy is non-ablative, insufficient to eliminate the tumor (kill all tumor cells). In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors.

In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT).

In some embodiments, the radiation therapy comprises administering a radiopharmaceutical. The radiopharmaceuticals can be delivered via any vehicle such as a cell, a protein, or a small molecule complex. In some embodiments, the radiopharmaceutical is administered to the tumor tissue. See e.g., Sgouros et al. Radiopharmaceutical therapy in cancer: clinical advances and challenges. Nat Rev Drug Discov 19, 589-608 (2020).

In some embodiments, the radiation therapy is brachytherapy, optionally comprising interstitial brachytherapy, intracavitary brachytherapy, intraluminal radiation therapy, and radioactively tagged molecules given intravenously.

### STING Activator

In some embodiments, the pro-inflammatory agent comprises or is a STING activator.

Stimulator of IFN genes (STING, also known as TMEM173, MITA, MPYS or ERIS) is a pattern recognition receptor (PRR) that recognizes cytosolic DNA in the form of cyclic dinucleotides (CDNs), such as the bacterial product cyclic-guanosine monophosphate-adenosine monophosphate (3′3′ cGAMP). In addition to bacterial components, other forms of DNA from viruses, or the host cell, that find their way into the cytosol are recognized by an enzyme c-GMP-AMP (cGAMP) synthase (cGAS). Upon cytosolic DNA binding, cGAS converts ATP and GTP into the metazoan-specific CDN 2′3′-cGAMP for STING recognition and activation. STING is a transmembrane protein that exists as dimers anchored within the endoplasmic reticulum membrane and forms a V-shaped pocket that enables cytosolic CDN binding. Ligand binding results in significant conformational changes in the C-terminal domain of STING, mediating its transport to Golgi compartments. At the Golgi, STING recruits TANK-binding kinase 1 (TBK1), which facilitates IRF3 phosphorylation, nuclear translocation and the strong induction of transcription of type I IFNs (e.g., IFN-β). STING also triggers a robust pro-inflammatory cytokine response [e.g., tumor necrosis factor (TNF)] by activating Nuclear Factor-kappa B (NF-κB) and this part of the pathway can be mediated independent of TBK1 via a closely related homologue protein, IKKE. See e.g., Peng et al., Front Immunol. 2022 Feb. 25; 13:794776; Amougezar et al., Cancers (Basel). 2021 May 30; 13 (11): 2695.

In some embodiments, the STING activator is a cyclic-guanosine monophosphate-adenosine monophosphate (cGAMP, e.g., 3′3′ cGAMP, e.g., 2′3′ cGAMP).

In some embodiments, the STING activator is a bacterial vector (e.g., SYNB1891, STACT-TREX-1).

In some embodiments, the STING activator is a CDN compounds (e.g., ADU-S100, BI-STING, BMS-986301, GSK532, JNJ-4412, MK-1454, SB11285, 3′3′-cyclic AIMP).

In some embodiments, the STING activator is a non-CDN small molecule (e.g., ALG-031048, E7755, JNJ-′6196, MK-2118, MSA-1, MSA-2, SNX281, SR-717, TAK676, TTI-10001).

In some embodiments, the STING activator is a nanovaccine (e.g., PC7A NP, cCAMP-NP, ONM-500).

In some embodiments, the STING activator is an antibody-drug conjugate (e.g., XMT-2056, CRD-5500).

Other exemplary STING activators can be found in Amougezar et al., Cancers (Basel). 2021 May 30; 13 (11): 2695, which is incorporated by reference here by its entirety.

### PAMP/DAMP Activators

In some embodiments, the pro-inflammatory agent comprises or is a PAMP/DAMP activator.

The organism senses microbial infection through innate receptors encoded in the genome, called pattern-recognition receptors, including the Toll-like receptors (TLRs), the nucleotide-binding and oligomerization domain (NOD)-like receptors, and retinoic acid-inducible gene I (RIG-I)-like receptors. These receptors recognize pathogen-associated molecular patterns (PAMPs) expressed by bacteria, fungi, and viruses, but also bind damage-associated molecular patterns (DAMPs), which are molecules released by sterile injury. Thus, PAMPs and DAMPs that bind to the same type of receptors initiate identical intracellular pathways terminating in identical effector functions. See e.g., Alisi et al., Hepatology. 2011 November; 54 (5): 1500-2.

In some embodiments, the pro-inflammatory agent is a PAMP activator. Exemplary PAMP activator includes triacyl lipopeptides, LPS, lipoprotein, peptidoglycan, zymosan, lipoteichoic acid, trypanosomal phospholipids, Pam3Cys porins, lipoarabinomannan, double-stranded RNA, poly(I:C), trepanosomal lipids, taxol, Pseudomonas exoenzyme S, RSV F protein, MMTV envelope protein, flagellin, diacyl lipopeptides, single-stranded RNA, imiquimod, single-stranded RNA, resquimod, bacterial/viral DNA, CpG DNA, ureobacteria, and toxoplasma LPS.

In some embodiments, the pro-inflammatory agent is a DAMP activator. Exemplary DAMP activator includes defensins, HSP60, HSP70, messenger RNA, low-molecular-weight hyaluronic acid, fibrinogen, fibronectin, fx1-defensin, heparan sulfate, HSP60, HSP70, HSP90, HMGB1, and unmethylated CpG DNA.

### Chemotherapeutic Agent

In some embodiments, the pro-inflammatory agent comprises or is a chemotherapeutic agent.

In some embodiments, the chemotherapeutic agent is an alkylating agent. Exemplary alkylating agents include nitrogen mustard (e.g., endamustine, cyclophosphamide, ifosfamide), nitrosoureas (e.g., carmustine, lomustine), platinum analogs (e.g., carboplatin, cisplatin, oxaliplatin), triazenes (e.g., dacarbazine, procarbazine, temozolamide), alkyl sulfonate (e.g., busulfan), and ethyleneimine (e.g., thiotepa).

In some embodiments, the chemotherapeutic agent is an antimetabolite. Exemplary antimetabolites include cytidine analogs (e.g., azacitidine, decitabine, cytarabine, gemcitabine), folate antagonists (e.g., methotrexate, pemetrexed), purine analogs (e.g., cladribine, clofarabine, nelarabine), pyrimidine analogs (e.g., fluorouracil (5-FU), capecitabine (prodrug of 5-FU)).

In some embodiments, the chemotherapeutic agent is an antimicrotubular agent. Exemplary antimmicrotubular agents include topoisomerase II inhibitors (e.g., anthracyclines, doxorubicin, daunorubicin, idarubicin, mitoxantrone), topoisomerase I inhibitors (e.g., irinotecan, topotecan), taxanes (e.g., paclitaxel, docetaxel, cabazitaxel), vinca alkaloids (e.g., vinblastine, vincristine, vinorelbine), antibiotics (e.g., actinomycin D, bleomycin, daunomycin).

Other exemplary chemotherapeutic agents include hydroxyurea, tretinoin, arsenic trioxide, and proteasome inhibitors (e.g., bortezomib).

### Pro-Inflammatory Cytokines

In some embodiments, the pro-inflammatory agent is a pro-inflammatory cytokine.

In some embodiments, the pro-inflammatory cytokine promotes the M1 macrophages. See e.g., Duque et al., Front Immunol. 2014; 5:491. In some embodiments, the pro-inflammatory cytokine comprises or is TNF, IFNγ, and/or GM-CSF.

In some embodiments, the pro-inflammatory cytokine comprises IL-6, TNFα, a cytokine from IL-1 family (e.g., IL-1a, IL-1β, IL-18, IL-33, and IL-36), and/or IFNγ.

In some embodiments, the pro-inflammatory cytokine comprises a cytokine from IL-1 family. In some embodiments, the pro-inflammatory cytokine comprises any one or more of IL-1α, IL-1β, IL-18, IL-33, and IL-36. See e.g., Sims, J., Smith, D. The IL-1 family: regulators of immunity. Nat Rev Immunol 10, 89-102 (2010).

### Checkpoint Inhibitors

In some embodiments, the pro-inflammatory agent is a checkpoint inhibitor. Immune checkpoints are pathways with inhibitory or stimulatory features that maintain self-tolerance and assist with immune response. The most well-described checkpoints are inhibitory in nature and include the cytotoxic T lymphocyte-associated molecule-4 (CTLA-4), programmed cell death receptor-1 (PD-1), and programmed cell death ligand-1 (PD-L1). See e.g., Marin-Acevedo et al., J Hematol Oncol 14, 45 (2021).

In some embodiments, the checkpoint inhibitor targets CLTA-4, PD-1 or PD-L1 (e.g., an antibody targeting CTLA-4, PD-1 or PD-L1).

In some embodiments, the checkpoint inhibitor targets LAG-3, TIM-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1 (M-CSF)/CSF-1R, IL-1/IL-1R3 (IL-1RAP), IL-8, SEMA4D, Ang-2, CLEVER-1, Axl, or phosphatidylserine.

In some embodiments, the checkpoint inhibitor comprises or is lipilimumab, Cemiplimab, Nivolumab, Pembrolizumab, Atezolizumab, Avelumab, Durvalumab, LAG525 (IMP701), REGN3767, BI 754,091, tebotelimab (MGD013), eftilagimod alpha (IMP321), FS118, MBG453, Sym023, TSR-022, MGC018, FPA150, EOS100850, AB928, CPI-006, Monalizumab, COM701, CM24, NEO-201, Defactinib, PF-04136309, MSC-1, Hu5F9-G4 (5F9), ALX148, TTI-662, RRx-001, Lanotuzumab (MCS110), LY3022855, SNDX-6352, Emactuzumab (RG7155), Pexidartinib (PLX3397), CAN04, Canakinumab (ACZ885), BMS-986253, Pepinemab (VX15/2503), Trebananib, FP-1305, Enapotamab vedotin (EnaV), or Bavituximab.

### Cancer Vaccine

In some embodiments, the pro-inflammatory agent comprises or is a cancer vaccine. Cancer vaccine stimulates anti-tumor immunity with tumor antigens, which could be delivered in the form of whole cells, peptides, nucleic acids, etc. Ideal cancer vaccines could overcome the immune suppression in tumors and induce both humoral immunity and cellular immunity.

In some embodiments, the cancer vaccine comprises a cell-based vaccine, a peptide-based vaccine, a viral-based vaccine, and/or a nucleic acid-based vaccine. See e.g., Liu et al., J Hematol Oncol 15, 28 (2022).

Cell-based vaccines are the form of cancer vaccines initially. Cell-based cancer vaccines are often prepared from whole cells or cell fragments, containing almost tumor antigens, inducing a broader antigen immune response. DC vaccine is an important branch of cell-based vaccines. Personalized neoantigen cancer vaccines based on DC have shown promising anti-tumor effects in clinical. Viruses are naturally immunogenic and their genetic material can be engineered to contain sequences encoding tumor antigens. Several recombinant viruses, such as adenovirus, can infect immune cells as vectors. The engineered virus vaccines can present tumor antigens in large quantities in the immune system and produce anti-tumor immunity. Furthermore, the oncolytic virus can be used as a vector as well. Except for providing tumor antigens, the virus itself can also lyse the tumor, release tumor antigens, further increase the vaccine's effectiveness, and produce long-term immune memory.

Peptide-based subunit vaccines, including chemical and biosynthetic preparations of predicted or known specific tumor antigens, induce a robust immune response against the particular tumor antigen site. Peptide-based subunit vaccine combined with adjuvants can efficiently provoke humoral immune response, suitable for preventing and treating viral infectious diseases.

HBV and HPV vaccines for liver and cervical cancers were primarily peptide-based subunit vaccines. Especially, virus-like particles (VLP)-based subunit vaccines that can activate cellular immune responses have shown good anti-tumor activity in recent years.

The nucleic acid vaccine induces strong MHC I mediated CD8+ T cell responses; thus, it is a desirable cancer vaccine platform. Nucleic acid vaccines can simultaneously deliver multiple antigens to trigger humoral and cellular immunity. Additionally, nucleic acid vaccines can encode full-length tumor antigens, allowing APC to cross-present various epitopes or present several antigens simultaneously. Finally, the nucleic acid vaccine preparation is simple and fast, which is suitable for developing personalized neoantigen cancer vaccines.

### Oncolytic Virus

In some embodiments, the pro-inflammatory agent is an oncolytic virus (OV). The oncolytic viruses (OVs) are organisms able to identify, infect, and lyse different cells in the tumor environment, aiming to stabilize and decrease the tumor progression. They can present a natural tropism to the cancer cells or be oriented genetically to identify specific targets. See e.g., Apolonio et al., World J Virol. 2021 Sep. 25; 10 (5): 229-255.

Oncolytic viruses represent an exciting new avenue of cancer therapy. Such viruses have the remarkable ability to hunt and terminate cancer cells while leaving healthy cells unharmed, as well as enhancing the immune system's ability to recognize and terminate cancer cells. See e.g., Cancer Cell. 2022 Aug. 15; S1535-6108 (22) 00357-9.

In some embodiments, the oncolytic virus comprises or is an adenovirus (e.g., ONYX-15, LOAd703 virus), a protoparvovirus, a parvovirus (e.g., H-1PV), a vaccinia virus (VACV), a Reovirus (e.g., Reolysin), or a Herpes simplex virus (HSV, e.g., HSV-1, HSV-2, G207, LIBR1, HF10, T-VEC, Orien X010).

Other exemplary oncolytic viruses include JX-593, Coxsackievirus A21 (CVA21), marabá virus or its MG1 variant, DNX2440 adenovirus, fowl pox virus, and Sendai virus.

### Cells

In some embodiments, the pro-inflammatory agent comprises cells that that trigger inflammatory factors. In some embodiments, the cells are tumor-infiltrating lymphocytes. In some embodiments, the cells specifically recognize a tumor antigen (e.g., being engineered to express a CAR recognizing a tumor antigen). In some embodiments, the cells are T cells. In some embodiments, the cells are CAR-T cells. In some embodiments, the cells are NK cells (e.g., CAR-NK cells). In some embodiments, the cells are neutrophils (e.g., CAR-expressing neutrophils cells). In some embodiments, the cells are TCR-T cells. In some embodiments, the cells are APCs (e.g., macrophages or dendritic cells). In some embodiments, the cells are CAR-macrophages or CAR-monocytes. In some embodiments, the cells are SIRPαnt-macrophages. In some embodiments, the cells are stem cells. In some embodiments, the cells are allogenic. In some embodiments, the cells are autologous.

### Immune Cells, Monocytes or Macrophages

Immune cells described herein encompass various kinds of immune cells.

In some embodiments, the immune cells comprise monocytes or macrophages described herein. In some embodiments, the macrophages are identified by F4/80 expression. In some embodiments, the macrophages have a M1 phenotype. In some embodiments, at least 50%, 60%, 70%, 80%, 90%, 95%, 98%, or 99% of the macrophages in the immune cells have a M1 phenotype.

In some embodiments, the macrophages are engineered to be deficient in tyrosine kinase inhibitor expression and/or activation. In some embodiments, the monocytes or macrophages express a reduced level of tyrosine kinase for at least a period of time (e.g., for at least 1, 2, 3, 4, or 5 days) or are resistant to activation for at least a period of time (e.g., for at least 1, 2, 3, 4, or 5 days). In some embodiments, the period of time is no more than about 10, 9, 8, 7, 6, 5, 4, or 3 days.

In some embodiments, the monocytes or macrophages have reduced tyrosine kinase activity for no more than about 5 consecutive days (e.g., for no more than 5, 4, or 3 days) before the tyrosine kinase activity level returns to normal.

Methods to engineer monocytes or macrophages to transiently express a reduced level of tyrosine kinase are well-known in the field. Exemplary methods include contacting the monocytes or macrophages with a tyrosine kinase inhibitor described herein (such as a small molecule, a nucleic acid (e.g., a siRNA, a shRNA, an antisense RNA, a microRNA), a nucleic acid editing system (e.g., a CRISPR system), and a protein agent (e.g., an antibody agent that targets tyrosine kinases or activated tyrosine kinase)) in vivo or in vitro.

In some embodiments, the immune cells comprise T cells (e.g., CAR-T cells).

In some embodiments, the immune cells comprise NK cells (e.g., CAR-NK cells).

In some embodiments, the immune cells comprise neutrophils (e.g., CAR-expressing neutrophils cells).

In some embodiments, the immune cells comprise antigen presenting cells (APCs, e.g., dendritic cells).

In some embodiments, the immune cells are derived from the same individual (i.e., autologous). In some embodiments, the immune cells are allogeneic.

In some embodiments, the immune cells are engineered to express a chimeric antigen receptor, optionally wherein the chimeric antigen receptor specifically binds to a tumor antigen.

In some embodiments, the immune cells express a high level of MHC-I, MHC-II, CD80 and/or CD86. In some embodiments, the immune cells express a high level of MHC-I, MHC-II, CD80 and/or CD86 when the expression level of MHC-I, MHC-II, CD80 and/or CD86 on the immune cells is comparable (e.g., at least more than 50%) of that on activated antigen presenting cells (APCs).

In some embodiments, the immune cells express one or more pro-inflammatory cytokines, optionally wherein the one or more pro-inflammatory cytokines comprise TNFα and/or IL-12.

In some embodiments, the immune cells do not express a significant level of TGFβ and/or IL-10.

In some embodiments, the tyrosine kinase inhibitor and the immune cells are administered within 24 hours (e.g., 12 hours, 8 hours, 4 hours, 2 hours, 1 hour, or 0.5 hour) of each other, optionally wherein the tyrosine kinase inhibitor and the immune cells are administered within 4 hours of each other.

In some embodiments, the tyrosine kinase inhibitor, the immune cells, and a pro-inflammatory agent described above are administered within 24 hours (e.g., 12 hours, 8 hours, 4 hours, 2 hours, 1 hour, or 0.5 hour) of each other. In some embodiments, the immune cells are administered simultaneously or concurrently with the tyrosine kinase inhibitor and/or the pro-inflammatory agent.

### Inflammation Reaction or Ongoing Infection

There has been a body of evidence that both acute and chronic inflammation are associated with the development and progression of cancer. Progress in research on inflammation revealed a connection between inflammatory processes and neoplastic transformation, the progression of tumor, and the development of metastases and recurrences. Moreover, the tumor invasive procedures (both surgery and biopsy) affect the remaining tumor cells by increasing their survival, proliferation and migration. One of the concepts explaining this phenomenon is an induction of a wound healing response. While in normal tissue it is necessary for tissue repair, in tumor tissue, induction of adaptive and innate immune response related to wound healing, stimulates tumor cell survival, angiogenesis and extravasation of circulating tumor cells. See, e.g., Singh et al., Ann Afr Med. 2019 July-September; 18 (3): 121-126; Piotrowski et al., Rep Pract Oncol Radiother. 2020 May-June; 25 (3): 422-427.

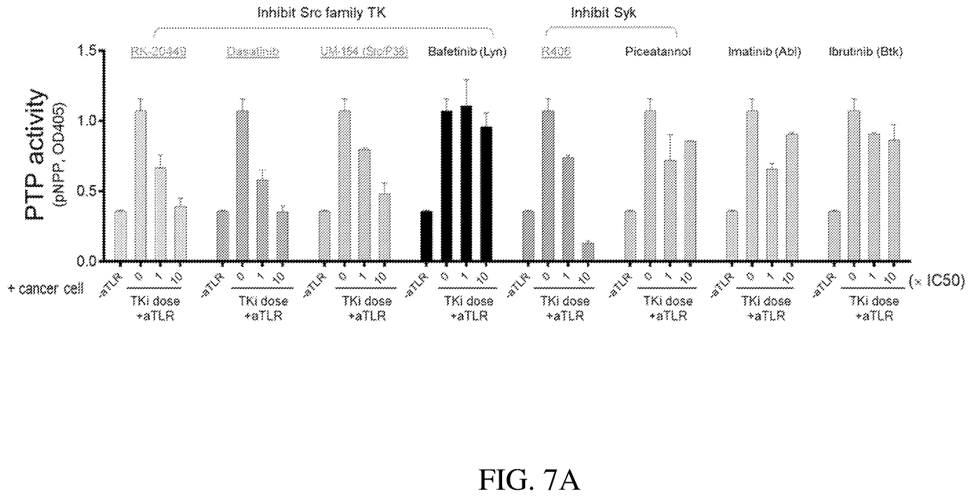

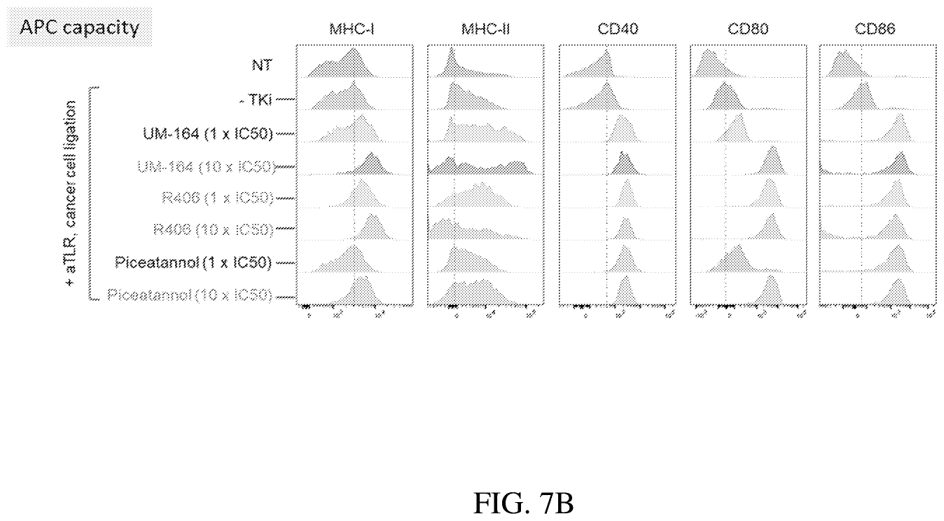

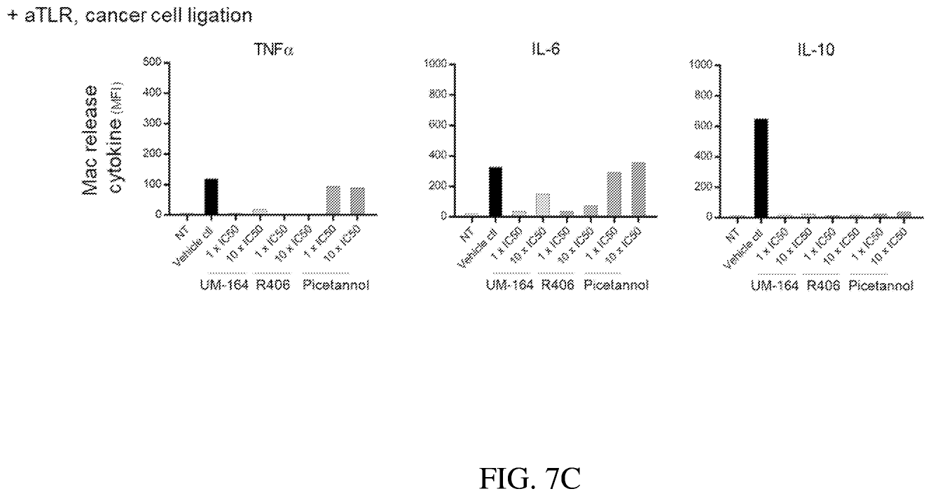

However, as demonstrated in this application, a combined use of a tyrosine kinase inhibitor and a pro-inflammatory agent unleashes proinflammatory response and transforms an immunosuppressive tumor environment into a place with an inflammation signature. See e.g., FIG. 7F. Remarkable anti-tumor effects were achieved. These results provide support for using methods described herein for treating an individual who is in under an inflammation reaction.

In some embodiments, the individual is under an inflammation reaction or has an ongoing infection when being treated with the methods described herein. The inflammation reaction described herein can be reflected by, e.g., a) an increase in one or more (e.g., at least one, two, three, four, five) inflammatory cytokines (such as IFNγ, IL-12b, TNFα, IL-6, IL-1b, IFN-a1, IFN-a2, IFN-b1), b) a decrease in one or more (e.g., at least one, two or three) anti-inflammatory cytokine (such as TGFb1, TGFb2, TGFb3), c) an increase in the infiltrating immune cells (such as T cells, NK cells, macrophages, neutrophils), d) a decrease in suppressive immune cells (such as MDSCs), and/or e) an increase in one or more (e.g., at least one, two, three, four, or five) immunogenic co-stimulatory molecules (such as CD80, CD86, OX40L, CD40, ICOS-L, PD-L1, GITRL) in the tissue (e.g., tumor tissue) or immune cells (such as macrophages).

In some embodiments, the inflammation reaction is an acute inflammation reaction.

In some embodiments, the inflammation reaction is in the tumor. In some embodiments, the inflammation reaction is at a site distinct from the tumor.

In some embodiments, there is an inflammation reaction where there are at least two (e.g., two, three, four or five events) selected from the group consisting of a) an increase in one or more (e.g., at least one, two, three, four, five) inflammatory cytokines (such as IFNγ, IL-12b, TNFα, IL-6, IL-1b, IFN-a1, IFN-a2, IFN-b1), b) a decrease in one or more (e.g., at least one, two or three) anti-inflammatory cytokine (such as TGFb1, TGFb2, TGFb3), c) an increase in the infiltrating immune cells (such as T cells, NK cells, macrophages, neutrophils), d) a decrease in suppressive immune cells (such as MDSCs), and/or e) an increase in one or more (e.g., at least one, two, three, four, or five) immunogenic co-stimulatory molecules (such as CD80, CD86, OX40L, CD40, ICOS-L, PD-L1, GITRL) in the tissue (e.g., tumor tissue) or immune cells (such as macrophages).

In some embodiments, the increase described herein refers to at least about 20%, 30%, 40%, 50%, 60%, 70%, 80%, 90%, 100%, 125%, 150%, 175%, or 200% more in the amount of level as compared to that in a reference state, optionally wherein the reference state is when the individual is neither treated with the methods described herein nor infected by a pathogen. In some embodiments, the increase described herein refers to at least about 5-fold, 10-fold, 20-fold, 30-fold, 40-fold, 50-fold, 60-fold, 70-fold, 80-fold, 90-fold, 100-fold, 150-fold, 200-fold, 250-fold, 500-fold, or 1000-fold more in the amount of level as compared to that in a reference state, optionally wherein the reference state is when the individual is neither treated with the methods described herein nor infected by a pathogen. In some embodiments, the reference state is when a healthy individual is not infected by a pathogen.

In some embodiments, the decrease described herein refers to at least about 10%, 20%, 30%, 40%, 50%, 60%, 70%, 80%, 90%, 95%, 98%, 99%, or 99.9% less in the amount of level as compared to that in a reference state, optionally wherein the reference state is when the individual is neither treated with the methods described herein nor infected by a pathogen. In some embodiments, the reference state is when a healthy individual is not infected by a pathogen.

In some embodiments, the individual has an inflammation reaction (e.g., in the tumor, e.g., in a site distinct from the tumor) within about one week, 6 days, 5 days, 4 days, 3 days, 2 days, or one day prior to and/or after the administration of the tyrosine kinase inhibitor.

In some embodiments, the individual has an ongoing inflammation reaction (e.g., in the tumor, e.g., in a site distinct from the tumor) when the tyrosine kinase inhibitor is administered.

In some embodiments, the individual has an ongoing infection when the tyrosine kinase inhibitor is administered. In some embodiments, the method further comprises assessing the presence of an infection in the individual, e.g., an infection associated with a virus, a fungus, and/or a bacteria.

### Immunogenic Cell Death

In some embodiments, the individual has immunogenic cell death when being treated with the methods described herein.

Immunogenic cell death (ICD) is a type of cancer cell death that can be induced by different stressors, including but not limited to (1) intracellular pathogens; (2) conventional chemotherapeutics such as anthracyclines, DNA-damaging agents, and proteasomal inhibitors; (3) targeted anticancer agents such as the tyrosine kinase inhibitor crizotinib, the epidermal growth factor receptor-specific monoclonal antibody cetuximab and poly-ADP-ribose polymerase (PARP) inhibitors; and (4) numerous physical modalities, encompassing hypericin- and redaporfin-based photodynamic therapy, extracorporeal photochemotherapy, various forms of ionizing radiation, high hydrostatic pressure, and severe heat shock. It involves the activation of the immune system against cancer in immunocompetent hosts. ICD comprises the release of damage-associated molecular patterns (DAMPs) from dying tumor cells that result in the activation of tumor-specific immune responses, thus eliciting long-term efficacy of anticancer drugs by combining direct cancer cell killing and antitumor immunity. DAMPs include the cell surface exposure of calreticulin (CRT) and heat-shock proteins (HSP70 and HSP90), extracellular release of adenosine triphosphate (ATP), high-mobility group box-1 (HMGB1), type I IFNs and members of the IL-1 cytokine family. See e.g., Ahmed et al., Mol Oncol. 2020 December; 14 (12): 2994-3006 and Fucikova et al., Cell Death Dis. 2020 Nov. 26; 11 (11): 1013.

Key DAMPs for cell death to be perceived as immunogenic include calreticulin, high-mobility group box 1 (HMGB1), ATP, annexin A1 (ANXA1), and type I IFN. The main hallmarks of immunogenic cell death (ICD) can be assessed by flow cytometry, (immuno) fluorescence microscopy, immunoblotting, or luminometry, based on a variety of different approaches. See e.g., Cell Death Dis. 2020 Nov. 26; 11 (11): 1013.

In some embodiments, the individual has ICD (e.g., in the tumor, e.g., in a site distinct from the tumor) within about one week, 6 days, 5 days, 4 days, 3 days, 2 days, or one day prior to and/or after the administration of the tyrosine kinase inhibitor.

In some embodiments, the individual has ongoing ICD (e.g., in the tumor, e.g., in a site distinct from the tumor) when the tyrosine kinase inhibitor is administered.

In some embodiments, the individual has ICD when a sample from the cancer has a higher level of one or more (e.g., at least 10%, 20%, 30%, 40%, 50%, 60%, 70%, 80%, 90% more) DAMPs than a reference sample (e.g., a corresponding sample in a healthy control, e.g., a sample from the cancer prior to the administration of a therapy that induces ICD. In some embodiments, the DAMPs are selected from the group consisting of endoplasmic reticulum (ER) chaperones (e.g., calreticulin (CALR), e.g., heat-shock proteins (HSPs)), the non-histone chromatin-binding protein high-mobility group box 1 (HMGB1), the cytoplasmic protein annexin A1 (ANXA1), and the small metabolite ATP, and type I interferons (IFNs).

### Individuals

In some embodiments, the individual has a solid tumor. In some embodiments, the individual has a hematologic cancer.

In some embodiments, the individual has an advanced cancer. In some embodiments, the individual has a late stage cancer. In some embodiments, the individual has a malignant cancer. In some embodiments, the individual has a cancer that is in stage II, III or IV. In some embodiments, the individual has an inoperable tumor and/or metastases. In some embodiments, the individual is a terminally ill individual.

In some embodiments, the individual has been subjected (e.g., within 1, 2, 4, 8, 12, 16, 20, or 24 hours, e.g., within 1, 2, 3, 4, 5, 6 or 7 days before the administration of the tyrosine kinase inhibitor) to a therapy that induces an inflammation reaction or an immunogenic cell death (e.g., radiotherapy). In some embodiments, the individual is to be subjected to (e.g., within 1, 2, 4, 8, 12, 16, 20, or 24 hours, e.g., within 1, 2, 3, 4, 5, 6 or 7 days after the administration of the tyrosine kinase inhibitor) a therapy that induces an inflammation reaction or an immunogenic cell death (e.g., radiotherapy).

In some embodiments, the individual has been subjected (e.g., within 1, 2, 4, 8, 12, 16, 20, or 24 hours, e.g., within 1, 2, 3, 4, 5, 6 or 7 days before the administration of the tyrosine kinase inhibitor) to a pro-inflammatory agent (such as any of the pro-inflammatory agents described herein). In some embodiments, the individual is to be subjected to (e.g., within 1, 2, 4, 8, 12, 16, 20, or 24 hours, e.g., within 1, 2, 3, 4, 5, 6 or 7 days after the administration of the tyrosine kinase inhibitor) a pro-inflammatory agent (such as any of the pro-inflammatory agents described herein).

In some embodiments, the individual does not have an autoimmune disease.

In some embodiments, the individual is a female. In some embodiments, the individual is a male.

In some embodiments, the individual is a human. In some embodiments, the individual is at least about 50, 55, 60, 65, 70 or 75 years old.

In some embodiments, the individual is selected for treatment based upon a high expression level and/or a high activation level of tyrosine kinases in the tumor tissue. In some embodiments, the individual has a high expression level and/or a high activation level of tyrosine kinase when the expression level and/or the activation level is at least about 20%, 30%, 40%, 50%, 60%, 70%, 80%, 90%, 100%, 125%, 150%, 175%, or 200% more than a reference expression level and/or a reference activation level of tyrosine kinase. In some embodiments, the individual has a high expression level and/or a high activation level of TYROSINE KINASE when the expression level and/or the activation level is at least about 5-fold, 10-fold, 20-fold, 30-fold, 40-fold, 50-fold, 60-fold, 70-fold, 80-fold, 90-fold, 100-fold, 150-fold, 200-fold, 250-fold, 500-fold, or 1000-fold more than a reference expression level and/or a reference activation level of tyrosine kinases. In some embodiments, the reference expression level or the reference activation level of tyrosine kinases is the corresponding expression or activation level of tyrosine kinases in a reference state, wherein the individual is not treated with a pro-inflammatory agent (or any immune therapy).

In some embodiments, the individual is at risk of developing systemic inflammation and/or CRS. In some embodiments, the individual develops systemic inflammation and/or CRS prior to the administration of an agent that reduces systemic inflammation (e.g., a TNFα inhibitor, e.g., an anti-TNFα antibody). Cytokine release syndrome can damage or cause organ failure in most organ systems. For example, organs that can become damaged due to CRS may include, but are not limited to, the lungs, the kidneys, the liver, the brain, the heart, the spleen, or any combination thereof, for example multi-organ failure.

In some embodiments, the individual is administered an agent that reduces systemic inflammation (e.g., a TNFα inhibitor, e.g., an anti-TNFα antibody). In some embodiments, the administration occurs prior to the development of systemic inflammation in the individual. In some embodiments, the individual develops mild cytokine release syndrome. In some embodiments, the individual develops CRS of grade 1. Mild symptoms of CRS can include fever, fatigue, headache, rash, arthralgia, and myalgia. Mild CRS can be treated by treating the symptoms or by administration of anti-inflammatory drugs such as corticosteroids. Mild CRS can often be resolved within one to two weeks and does not require or necessitate hospitalization.

In some embodiments, the individual does not develop severe cytokine release syndrome. In some embodiments, the individual does not develop CRS of grade 2. In some embodiments, the individual does not develop CRS of grade 3. In some embodiments, the individual does not develop CRS of grade 4. More severe cases are characterized by hypotension and high fever, and severe CRS can progress to an uncontrolled systemic inflammatory response with vasopressor-requiring circulatory shock, vascular leakage, disseminated intravascular coagulation, and multi-organ system failure. More severe cases of CRS often require hospitalization of symptoms. Laboratory abnormalities that are common in patients with CRS include cytopenias, elevated creatinine and liver enzymes, deranged coagulation parameters, and a high CRP. There are four grading systems currently used for cytokine release syndrome, as shown in Table 1 below. See, e.g., Liu, D. and Zhao, J., J Hematol Oncol. 2018 Sep. 24; 11 (1): 121; and Shimabukuro-Vornhagen, A. et al., J Immunother Cancer. 2018 Jun. 15; 6 (1): 56, hereby incorporated by reference in their entirety.

In some embodiments, the individual has developed CRS prior to administration of an agent that reduces systemic inflammation (e.g., a TNFα inhibitor, e.g., an anti-TNFα antibody). In some embodiments, the individual has developed CRS of grade 1. In some embodiments, the individual has developed CRS of grade 2. In some embodiments, the individual has developed CRS of grade 3. In some embodiments, the individual has developed CRS of grade 4. In some embodiments, the individual who has developed CRS is administered an agent that reduces systemic inflammation (e.g., a TNFα inhibitor, e.g., an anti-TNFα antibody). In some embodiments, the agent that reduces systemic inflammation (e.g., a TNFα inhibitor, e.g., an anti-TNFα antibody) ameliorates, eliminates, or reverses the CRS, including organ damage, for example pro-inflammatory organ damage (e.g., nephritis, hepatitis, pneumonitis, myocarditis, appendicitis).

TABLE 1
Cytokine release syndrome medical grading systems.

| | Penn grading scale | CTCAE v4.0 | 2014 Lee et al. | MDACC |
|---|---|---|---|---|
| Grade | Mild reaction: treated | Mild reaction; | Symptoms are | Temperature ≥38° |
| 1 | with supportive care such | infusion | not life- | C. (fever) or |
| | as antipyretics and | interruption not | threatening and | grade 1 organ |
| | antiemetics | indicated; | require | toxicity |
| | | intervention not | symptomatic | |
| | | indicated | treatment only, | |
| | | | e.g., fever, | |
| | | | nausea, fatigue, | |
| | | | headache, | |
| | | | myalgias, and | |
| | | | malaise | |
| Grade | Moderate reaction: some | Therapy or | Symptoms | Systolic blood |
| 2 | signs of organ | infusion | require and | pressure <90 |
| | dysfunction (e.g., grade 2 | interruption | respond to | mmHg |
| | creatinine or grade 3 | indicated but | moderate | (hypotension) |
| | LFTs) related to CRS and | responds | intervention. | but responds to |
| | not attributable to any | promptly to | Oxygen | IV fluids or low- |
| | other condition. | symptomatic | requirement <40% | dose |
| | Hospitalization for | treatment (e.g., | or hypotension | vasopressors or |
| | management of CRS- | antihistamines, | responsive to | needing oxygen |
| | related symptoms, | NSAIDs, | fluids or low | (FiO2 <40%) |
| | including fevers with | narcotics, IV | dose pressors or | for SaO2 >90% |
| | associated neutropenia | fluids); | grade 2 organ | (hypoxia) or |
| | and need for IV therapies | prophylactic | toxicity | grade 2 organ |
| | (not including fluid | medications | | toxicity |
| | resuscitation for | indicated | | |
| | hypotension) | for ≤24 h | | |
| Grade | More severe reaction: | Prolonged | Symptoms | Systolic blood |
| 3 | hospitalization required | reaction (e.g., | require and | pressure <90 |
| | for management of | not rapidly | respond to | mmHg |
| | symptoms related to | responsive to | aggressive | (hypotension) |
| | organ dysfunction, | symptomatic | intervention. | and needs high- |
| | including grade 4 LFTs or | medication | Oxygen | dose or multiple |
| | grade 3 creatinine related | and/or brief | requirement ≥40% | vasopressors or |
| | to CRS and not | interruption of | or hypotension | needing |
| | attributable to any other | infusion); | requiring high- | oxygen(FiO2 ≥40%) |
| | conditions; this excludes | recurrence of | dose or multiple | for SaO2 >90% |
| | management of fever or | symptoms | pressors or grade | (hypoxia) or |
| | myalgias; includes | following initial | 3 organ toxicity | grade 3 organ |
| | hypotension treated with | improvement; | or grade 4 | toxicity or grade |
| | IV fluids (defined as | indicated for | transaminitis | 4 transaminitis |
| | multiple fluid boluses for | clinical sequelae | | |
| | blood pressure support) or | (e.g., renal | | |
| | low-dose vasopressors, | impairment, | | |
| | coagulopathy requiring | pulmonary | | |
| | fresh frozen plasma or | infiltrates) | | |
| | cryoprecipitate or | | | |
| | fibrinogen concentrate, | | | |
| | and hypoxia requiring | | | |
| | supplemental oxygen | | | |
| | (nasal cannula oxygen, | | | |
| | high-flow oxygen, CPAP, | | | |
| | or BiPAP). Patients | | | |
| | admitted for management | | | |
| | of suspected infection due | | | |
| | to fevers and/or | | | |
| | neutropenia may have | | | |
| | grade 2 CRS | | | |
| Grade | Life-threatening | Life-threatening | Life-threatening | Life-threatening |
| 4 | complications such as | consequences; | symptoms. | hypotension or |
| | hypotension requiring | pressor or | Requirements | needing |
| | high dose vasopressors | ventilator | for ventilator | ventilator |
| | and a hypoxia requiring | support | support or grade | support or grade |
| | mechanical ventilation | indicated | 4 oxygen | 4 oxygen |
| | | | toxicity | toxicity |
| | | | (excluding | (excluding |
| | | | transaminitis) | transaminitis) |

In some embodiments, the individual does not develop cytokine storm. In some embodiments, the individual develops mild cytokine storm. In some embodiments, the individual does not develop severe or life-threatening cytokine storm. Cytokine storm appears to be mainly a result of non-specific T cell activation, whereas CRS is more often a direct consequence of antigen-specific T cell activation. The clinical manifestations of cytokine storm and CRS can be similar (Liu, D. and Zhao, J., J Hematol Oncol. 2018 Sep. 24; 11 (1): 121).

### Cancer

Cancer described here can be any type or kind. In some embodiments, the cancer is a solid tumor. In some embodiments, the cancer is a hematologic cancer.

In some embodiments, the cancer is an advanced cancer. In some embodiments, the cancer is a late stage cancer. In some embodiments, the cancer is a terminal cancer. In some embodiments, the cancer is in stage II, III or IV. In some embodiments, the cancer is an inoperable tumor and/or is malignant.

In some embodiments, the tumor is at least 0.2 cm, 0.4 cm, 0.6 cm, 0.8 cm, 1 cm, 2 cm, 3 cm, 4 cm or 5 cm in length.

Examples of cancers described herein include, but are not limited to, adrenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, melanoma, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), and Meigs' syndrome.

In some embodiments, the cancer is a virus-infection-related cancer. In some embodiments, the cancer is a human papillomavirus (HPV)-related cancer (e.g., HPV-related cervical cancer, e.g., HPV-related head and neck cancer, e.g., HPV related squamous cell carcinoma). In some embodiments, the cancer is human herpes virus 8 (HHV8) related cancer (e.g., Kaposi sarcoma). In some embodiments, the cancer is human T-lymphotrophic virus (HTLV-1)-related cancer (e.g., adult T cell leukemia or lymphoma). In some embodiments, the cancer is Epstein-Barr virus (EBV) related cancer (e.g., Burkitt lymphoma, Hodgkin's and non-Hodgkin's lymphoma, stomach cancer). In some embodiments, the cancer is hepatitis B virus (HBV) related cancer (e.g., liver cancer). In some embodiments, the cancer is hepatitis C virus) related cancer (e.g., liver cancer, non-Hodgkin's lymphoma).

In some embodiments, the cancer is a liver cancer, a kidney cancer, an endometrial cancer, a thymic epithelial neoplasma, lung cancer, spindle cell sarcoma, chondrosarcoma, uterine smooth muscle, colon cancer, or pancreatic cancer.

In some embodiments, the cancer has been subjected to and/or failed one or more prior therapy (e.g., an immune checkpoint blockage therapy (e.g., a PD-1 antibody), a chemotherapy, a surgery, a cell therapy (e.g., an allogenic NK cell infusion therapy)).

In some embodiments, the cancer is a recurrent or refractory cancer.

In some embodiments, the cancer is refractory to one or more of irradiation therapy, chemotherapy, or immunotherapy (e.g., checkpoint blockade).

### Dosing, Method of Administration, and Delivery Vehicles

The tyrosine kinase inhibitor, the pro-inflammatory agent, and the immune cells (e.g., monocytes/macrophages) described herein can be administered at any desired dosage. Exemplary dosing regimens are described in e.g., “tyrosine kinases inhibitors” section.

In some aspects, the size of the dose in the pro-inflammatory agent, the tyrosine kinase inhibitor and/or the immune cells (e.g., monocytes/macrophages) is determined based on one or more criteria such as disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the activated immune cells being administered. For example, in some aspects, the number of monocytes or macrophages administered in the dose is determined based on the tumor burden that is present in the subject immediately prior to administration of the initiation of the dose of cells.

The pro-inflammatory agent, the tyrosine kinase inhibitor and/or the immune cells (e.g., monocytes/macrophages) can be administered by any suitable means, for example, by bolus infusion, by injection, e.g., intravenous or subcutaneous injections. In some embodiments, the pro-inflammatory agent, the tyrosine kinase inhibitor and/or the monocytes or macrophages are administered systemically (e.g., intravenously, subcutaneously, or intraperitoneally). In some embodiments, the pro-inflammatory agent, the tyrosine kinase inhibitor and/or the monocytes or macrophages are administered locally (e.g., intratumorally).

In some embodiments, the pro-inflammatory agent, the tyrosine kinase inhibitor and/or the immune cells (e.g., monocytes/macrophages) are administered by parenteral, intrapulmonary, and intranasal, and, if desired for local treatment, intralesional or intratumorally administration. Parenteral infusions include intramuscular, intravenous, intraarterial, intraperitoneal, or subcutaneous administration. In some embodiments, the pro-inflammatory agent and/or the tyrosine kinase inhibitor are administered orally.

In some embodiments, the immune cells (e.g., monocytes/macrophages) and the pro-inflammatory agent are administered simultaneously. In some embodiments the monocytes or macrophages and the pro-inflammatory agent are administered concurrently. In some embodiments, the immune cells (e.g., monocytes/macrophages) and the pro-inflammatory agent are administered sequentially. In some embodiments, the immune cells (e.g., monocytes/macrophages) and the pro-inflammatory agent are administered within about 7, 6, 5, 4, 3, 2, or 1 day. In some embodiments, the immune cells (e.g., monocytes/macrophages) and the pro-inflammatory agent are administered within about 24, 16, 12, 8, 4, 2, or 1 hour. In some embodiments, the immune cells (e.g., monocytes/macrophages) and the pro-inflammatory agent are administered within 30 minutes.

In some embodiments, the tyrosine kinase inhibitor and the pro-inflammatory agent are administered simultaneously. In some embodiments, the tyrosine kinase inhibitor and the pro-inflammatory agent are administered concurrently. In some embodiments, the tyrosine kinase inhibitor and the pro-inflammatory agent are administered sequentially. In some embodiments, the tyrosine kinase inhibitor and the pro-inflammatory agent are administered within about 7, 6, 5, 4, 3, 2, or 1 day. In some embodiments, the tyrosine kinase inhibitor and the pro-inflammatory agent are administered within about 24, 16, 12, 8, 4, 2, or 1 hour. In some embodiments, the tyrosine kinase inhibitor and the pro-inflammatory agent are administered within 30 minutes.

It is also contemplated that tyrosine kinase inhibitors and/or pro-inflammatory agents described herein can be delivered via any proper vehicles or methods. In some embodiments, the tyrosine kinase inhibitor and/or the pro-inflammatory agent are directly delivered into the tumor tissue. Different carrier systems can be utilized for this purpose. See e.g., Manzari et al. Targeted drug delivery strategies for precision medicines. Nat Rev Mater 6, 351-370 (2021); Tewabe et al., J Multidiscip Healthc. 2021; 14:1711-1724. In some embodiments, the tyrosine kinase inhibitor and/or the pro-inflammatory agent is delivered via a nanoparticle. In some embodiments, the tyrosine kinase inhibitor and/or the pro-inflammatory agent is delivered via a controlled release system. In some embodiments, the tyrosine kinase inhibitor and/or the pro-inflammatory agent is delivered via a biomaterial implant scaffold. In some embodiments, the tyrosine kinase inhibitor and/or the pro-inflammatory agent is delivered via an injectable biomaterial scaffold. In some embodiments, the tyrosine kinase inhibitor and/or the pro-inflammatory agent is delivered via a transdermal delivery system. See e.g., Riley et al., Nat Rev Drug Discov. 2019 March; 18 (3): 175-196.

In some embodiments, the tyrosine kinase inhibitor and/or the pro-inflammatory agent is delivered by a cell. See e.g., Millian et al., Ther Deliv. 2012 January; 3 (1): 25-41. In some embodiments, the cell comprises a macrophage. See e.g., Visser et al., Front Pharmacol. 2019 Jan. 25; 10:22. In some embodiments, the cell comprises a polymer encapsulated human retinal pigmented epithelial (aRPE) cell. See e.g., Nash et al., Clin Cancer Res. 2022 Aug. 22;CCR-22-1493. In some embodiments, the cells are encapsulated in a biocompatible material (e.g., biocompatible alginate capsules as discussed in Nash et al.)

In some embodiments, the tyrosine kinase inhibitor and/or the pro-inflammatory agent is associated with an antibody construct. In some embodiments, the tyrosine kinase inhibitor and/or the pro-inflammatory agent is connected with an antibody construct with via a linker (e.g., a cleavable linker). In some embodiments, the antibody construct specifically recognizes a tumor associated antigen. In some embodiments, the antibody construct comprises an antibody recognizing a tumor antigen. In some embodiments, the antibody construct is an antibody drug conjugate (ADC).

In some embodiments, the tyrosine kinase inhibitor and/or the pro-inflammatory agent is a delivered via a method or device that promotes delivery into a particular organ (e.g., the organ that has a tumor). See examples of these methods or devices in e.g., Alsaggar et al., J Drug Target. 2018 June-July; 26 (5-6): 385-397; Zhao et al., Cell. 2020 Apr. 2; 181 (1): 151-167, which are incorporated by reference in their entirety.

In embodiments, the tyrosine kinase inhibitor is delivered via a controlled drug delivery system (e.g., a slow release system or vehicle, e.g., a sustained release system or vehicle). Examples of such systems can be found in e.g., Adepu et al., Molecules. 2021 October; 26 (19): 5905; Oh et al., Chem. Asian J. 2022, 17, e202200333, which are incorporated by reference in their entirety.

### VI. Compositions Comprising the Tyrosine Kinase Inhibitor

The present application also provides compositions (e.g., pharmaceutical compositions) comprising the tyrosine kinase inhibitor, the pro-inflammatory agent, and/or the immune cells for treatment as described above.

In some embodiments, there is provided a composition (e.g., a pharmaceutical composition) comprising a tyrosine kinase inhibitor and a pro-inflammatory agent (such as any of the pro-inflammatory agents described here). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and a TLR agonist (e.g., CpG, polyI:C and/or R848). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and a STING activator (e.g., cGAMP, e.g., 2′3′-cGAMP, e.g., 3′3′-cGAMP). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and a chemotherapeutic agent (e.g., azathioprine (AZA), e.g., gemcitabine). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and a pro-inflammatory cytokine (e.g., IL-1b, IL-18, IL-6, and/or TNFα). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and a checkpoint inhibitor (e.g., an anti-PD-L1 antibody, an anti-PD-1 antibody or an anti-CLTA4 antibody). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and a bacteria component (e.g., LPS). In some embodiments, the composition further comprises a pharmaceutically acceptable carrier. In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and an agent that promotes immunogenic cell death (ICD). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and an agent used in a radiation therapy (such as any of the radiation therapy described herein). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and a PAMP/DAMP activator (such as any of the PAMP/DAMP activators described herein). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and a cancer vaccine (such as any of the cancer vaccines described herein). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and an oncolytic virus (such as any of the oncolytic viruses described herein). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and an agent used in a sound treatment (such as any of the sound treatments described herein). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and an agent used in a magnetic therapy (such as any of the magnetic therapies described herein). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and an agent used in electrical or electrochemical treatment (such as any of the electrical or electrochemical treatments described herein). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1 or an analog or a derivative thereof).

In some embodiments, there is provided a composition (e.g., a pharmaceutical composition) comprising a tyrosine kinase inhibitor and an agent used in an electrostatic treatment (such as any of the electrostatic treatments described herein). In some embodiments, the composition further comprises immune cells (such as monocytes or macrophages described herein). In some embodiments, the composition further comprises a pharmaceutically acceptable carrier. In some embodiments, the composition further comprises a SHP-1 inhibitor (such as TPI-1).

### Exemplary Embodiments

1. A method of treating a cancer in an individual, comprising administering to the individual a) a tyrosine kinase inhibitor, and b) a pro-inflammatory agent.
2. The method of embodiment 1, wherein the method comprises systemically administering the tyrosine kinase inhibitor.
3. The method of embodiment 1 or 2, wherein the pro-inflammatory agent comprises an agent selected from the group consisting of a TLR agonist, a STING activator, a PAMP/DAMP activator, a chemotherapy, a pro-inflammatory cytokine, a cancer vaccine, a bacteria component, a sound treatment, a magnetic therapy, an electrical treatment, and an electrostatic treatment.
4. A method of treating a cancer in an individual, comprising administering to the individual a tyrosine kinase inhibitor, wherein the individual is under an inflammation reaction.
5. The method of any one of embodiments 1-4, wherein the method further comprises administering the tyrosine kinase inhibitor to the individual intermittently.
6. The method of embodiment 5, wherein the method comprises administering the tyrosine kinase inhibitor is administered at least three times.
7. The method of embodiment 5 or embodiment 6, wherein the method comprises administering the tyrosine kinase inhibitor at an interval of no more than once every three days for at least twice.
8. The method of any one of embodiments 5-7, wherein the method comprises administering the tyrosine kinase inhibitor to the individual for at least two cycles, wherein each cycle has about three to about twenty days.
9. The method of any one of embodiments 1-8, wherein the tyrosine kinase inhibitor inhibits SHP-1 signaling.
10. The method of any one of embodiments 1-9, wherein the tyrosine kinase inhibitor has a half-life of no more than about 5 days, optionally the tyrosine kinase inhibitor has a half-life of no more than about 3 days.
11. The method of any one of embodiments 1-10, wherein the tyrosine kinase inhibitor is effective in inhibiting more than 50% of the tyrosine kinase activity for n more than about 5 days, optionally wherein the tyrosine kinase inhibitor is effective in inhibiting more than 50% of the tyrosine kinase activity for no more than about 3 days.
12. The method of any one of embodiments 1-11, wherein the tyrosine kinase inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets tyrosine kinase or activated tyrosine kinases).
13. The method of embodiment 12, wherein the tyrosine kinase inhibitor inhibits any one or more of: Src, Syk, Hck, Lck, Lyn, and Yes.
14. The method of embodiment 13, wherein the tyrosine kinase inhibitor is selected from the group consisting of RK-20449, Dasatinib, and R406.
15. The method of any one of embodiments 1-14, wherein the tyrosine kinase inhibitor is an inhibitor of a tyrosine kinase of a Src family.
16. The method of any one of embodiments 1-15, wherein the method comprises administrating the tyrosine kinase inhibitor systemically and locally, optionally wherein the method comprises intratumorally administering the tyrosine kinase inhibitor.
17. The method of any one of embodiments 2-16, wherein the systemic administration of a tyrosine kinase comprises oral administration, intravenous administration, subcutaneous administration, and/or intraperitoneal administration.
18. The method of any one of embodiments 1-3 and 5-17, wherein the pro-inflammatory agent and the tyrosine kinase inhibitor are administered within 24 hours of each other, optionally wherein the pro-inflammatory agent and the tyrosine kinase inhibitor are administered within 4 hours of each other.
19. The method of any one of embodiments 1-3 and 5-18, wherein the method comprises intratumorally administering the pro-inflammatory agent.
20. The method of any one of embodiments 1-3 and 5-19, wherein the method comprises administering the pro-inflammatory agent to a site that is different from the site of the cancer to be treated.
21. The method of any one of embodiments 1-2 and 5-18, wherein the pro-inflammatory agent comprises an agent selected from the group consisting of a TLR agonist, a STING activator, a radiation therapy, a PAMP/DAMP activator, a checkpoint inhibitor, a pro-inflammatory cytokine, a chemotherapeutic agent, a bacteria component, a cancer vaccine, an oncolytic virus, a sound treatment, a magnetic therapy, an electrical treatment, and an electrostatic treatment.
22. The method of any one of embodiments 1-3 and 5-21, wherein the pro-inflammatory agent comprises a TLR agonist.
23. The method of embodiment 22, wherein the TLR agonist activates a TLR on a macrophage, optionally wherein the TLR comprises TLR2, TLR3, TLR7, TLR8, and/or TLR9.
24. The method of embodiment 23, wherein the TLR agonist comprises CpG, polyI:C and/or R848.
25. The method of any one of embodiments 1-3 and 5-24, wherein the pro-inflammatory agent comprises a bacteria component, optionally the bacteria component comprises lipopolysaccharide (LPS).
26. The method of any one of embodiments 1-3 and 5-25, wherein the pro-inflammatory agent comprises a STING activator.
27. The method of embodiment 26, wherein the STING activator comprises 2′3′-cGAMP.
28. The method of any one of embodiments 1-3 and 5-27, wherein the pro-inflammatory agent comprises a chemotherapeutic agent.
29. The method of embodiment 28, wherein the chemotherapy comprises azathioprine (AZA).
30. The method of any one of embodiments 1-3 and 5-29, wherein the pro-inflammatory agent comprises a pro-inflammatory cytokine.
31. The method of embodiment 30, wherein the pro-inflammatory cytokine comprises IL-1b, IL-18, IL-6, and/or TNFα.
32. The method of any one of embodiments 1-3 and 5-31, wherein the pro-inflammatory agent comprises a radiation therapy.
33. The method of embodiment 32, wherein the radiation therapy comprises irradiation at site of the cancer to be treated.
34. The method of embodiment 32 or embodiment 33, wherein the radiation therapy comprises irradiation at a site that is different from the site of the cancer to be treated.
35. The method of any one of embodiments 32-34, wherein the dose of the radiation therapy is insufficient to kill tumor cells.
36. The method of any one of embodiments 1-3 and 5-35, wherein the pro-inflammatory agent comprises a checkpoint inhibitor.
37. The method of embodiment 36, wherein the checkpoint inhibitor comprises an anti-PD-L1 antibody, an anti-PD-1 antibody or an anti-CLTA4 antibody.
38. The method of any one of embodiments 1-3 and 5-37, wherein the pro-inflammatory agent is administered intermittently.
39. The method of any one of embodiments 1-3 and 5-38, wherein the pro-inflammatory agent and the tyrosine kinase inhibitor are administered simultaneously or concurrently.
40. The method of any one of embodiments 1-3 and 5-39, wherein the pro-inflammatory agent comprises immune cells.
41. The method of any one of embodiments 4-39, where the method further comprises administration of immune cells.
42. The method of embodiment 40 or 41, wherein the immune cells are derived from the same individual.
43. The method of any one of embodiments 40-42, wherein the immune cells comprise or are macrophages, optionally wherein the macrophages have a M1 phenotype.
44. The method of any one of embodiments 40-43, wherein the immune cells are derived from monocytes.
45. The method of any one of embodiments 40-44, wherein the immune cells express a high level of MHC-I, MHC-II, CD80 and/or CD86.
46. The method of any one of embodiments 40-45, wherein the immune cells express one or more pro-inflammatory cytokines, optionally wherein the one or more pro-inflammatory cytokines comprise TNFα and/or IL-12.
47. The method of any one of embodiments 40-46, wherein the immune cells do not express a significant level of TGFβ and/or IL-10.
48. The method of any one of embodiments 40-47, wherein the immune cells comprise T cells.
49. The method of any one of embodiments 40-48, wherein the immune cells are engineered to express a chimeric antigen receptor, optionally wherein the chimeric antigen receptor specifically binds to a tumor antigen.
50. The method of any one of embodiments 43-49, wherein the macrophages are engineered to be deficient in tyrosine kinases expression and/or activation.
51. The method of any one of embodiments 40-50, wherein the tyrosine kinase inhibitor and the immune cells are administered within 24 hours of each other, optionally wherein the tyrosine kinase inhibitor and the immune cells are administered within 4 hours of each other.
52. The method of any one of embodiments 40-51, wherein the immune cells are administered simultaneously or concurrently with the tyrosine kinase inhibitor.
53. The method of any one of embodiments 1-52, further comprising administering to the individual an effective amount of a SHP-1 inhibitor.
54. The method of any one of embodiments 1-53, further comprising administering to the individual an effective amount of an anti-TNFα antibody.
55. The method of any one of embodiments 1-54, wherein the cancer is a solid tumor.
56. The method of any one of embodiments 1-54, wherein the cancer is a hematological cancer.
57. The method of any one of embodiments 1-56, wherein the cancer is a late-stage cancer.
58. The method of any one of embodiments 1-57, wherein the cancer is resistant or refractory to a radiation therapy, a chemotherapeutic agent, and/or a checkpoint inhibitor.
59. The method of any one of embodiments 1-58, wherein the individual is a human.
60. A composition comprising a tyrosine kinase inhibitor and a pro-inflammatory agent, optionally wherein the pro-inflammatory agent comprises an agent selected from the group consisting of immune cells, a TLR agonist, a STING activator, an agent used in radiation therapy, a PAMP/DAMP activator, a checkpoint inhibitor, a pro-inflammatory cytokine, a chemotherapeutic agent, a bacteria component, a cancer vaccine, an oncolytic virus, and an agent used in sound treatment, a magnetic therapy, an electrical treatment or an electrostatic treatment.
61. The composition of embodiment 60, further comprising a SHP-1 inhibitor.

### Examples

The examples below are intended to be purely exemplary of the invention and should therefore not be considered to limit the invention in any way. The following examples and detailed description are offered by way of illustration and not by way of limitation.

### Example 1. RK-20449 and Dasatinib are Effective in a Tumor-Relevance Macrophage (TAM) Activation Model and Tumor Treatment in Syngeneic Murine Tumor Models

In vitro assays. The experiments tested effects of RK-20449 & Dasatinib on human and murine macrophages responding to TLR stimulation in the presence of cancer cells.

Human monocyte-derived macrophages (M) were pre-treated with RK-20449 and Dastinib for 15 minutes followed by addition of human SW620 colorectal cancer cells (2:1 ratio to M) and a mixture of TLR agonists (αTLR:CpG, PolyIC and R848, each 20 μg/ml). After incubation for 15 minutes at 37° C., cells were gently washed to remove the majority of SW620, followed by lysis of macrophage using Hank's buffer (pH 7.2) containing 1% Triton and 1 mM PMSF. Protein tyrosine phosphatase (PTP) activity was assayed with 10 mM in Hank's buffer at 37° C.

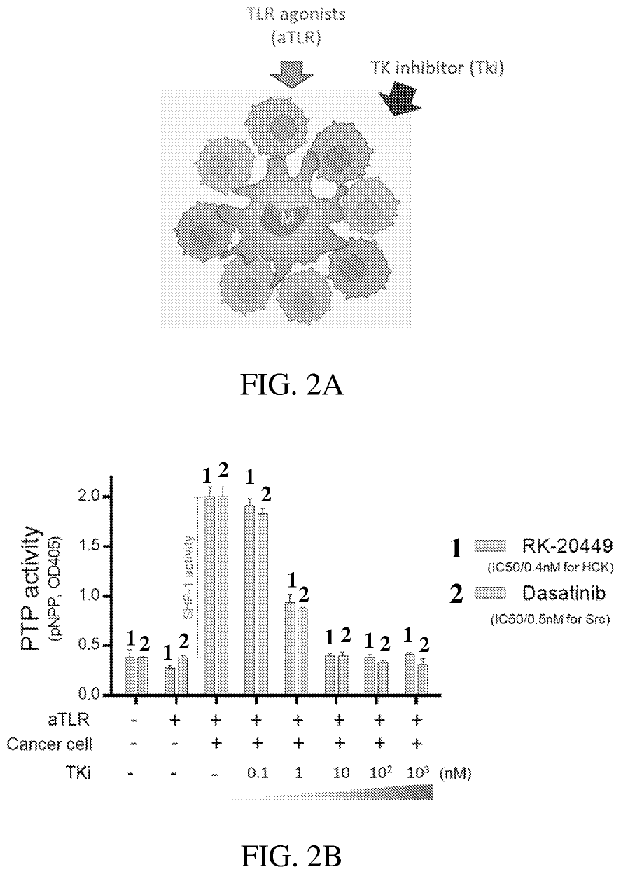

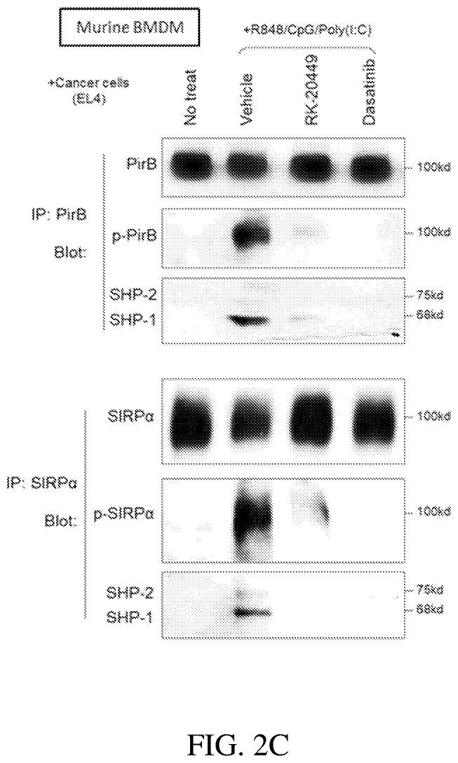

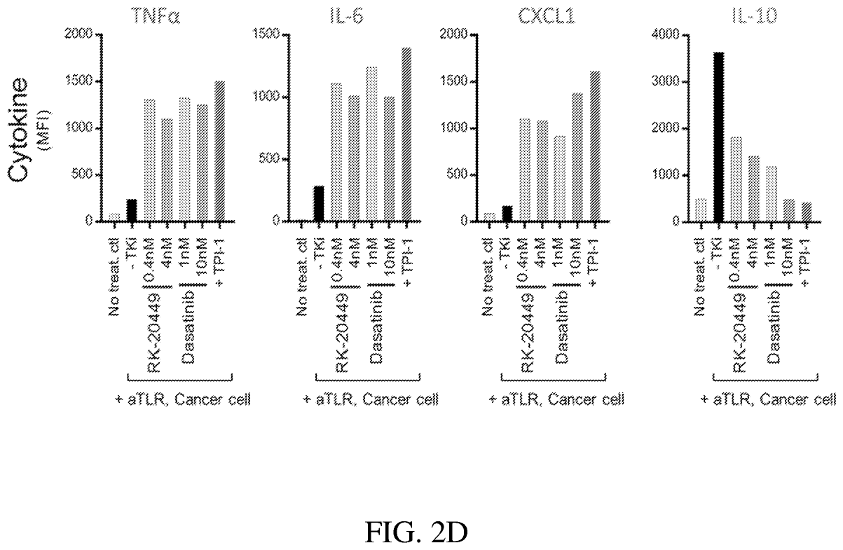

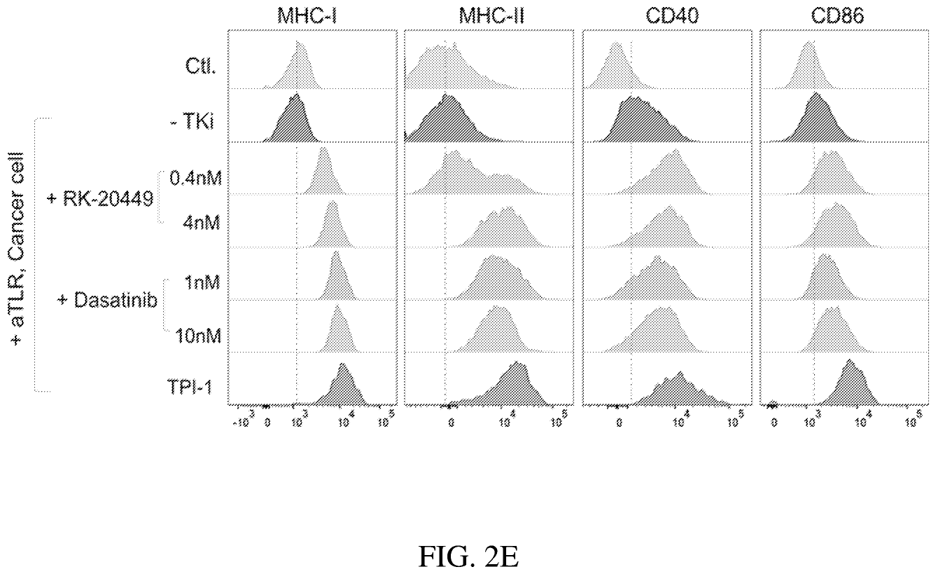

As shown in FIG. 2B, RK-20449 and Dasatinib dose-dependently diminished macrophage SHP-1 activity induced by TLR agonists and cancer cell ligation. The presence of cancer cell ligation facilitates phosphorylation of iRs in cytoplasmic ITIMs by TLR agonist-induced tyrosine kinase. FIG. 2C shows that RK20449 and Dasatinib depleted TLR agonist- and cancer ligation-induced iR phosphorylation and binding to SHP-1. Two iRs, SIRPα and PirB, in murine bone-marrow-derived macrophages were tested for phosphorylation and binding to SHP-1. FIGS. 2D and 2E further show that RK20449 and Dasatinib treatment enabled macrophage to overcome tumor cell-imposed inhibition to unleash proinflammatory phenotypic expression induced by TLR agonist, demonstrating marked increases in production of proinflammatory cytokines TNFα, IL-6, ad CXCL1 (FIG. 2D), and expression of cell surface antigen presentation machinery (FIG. 2E). The SHP-1 inhibitor TIP-1, which also demonstrated similar effects, was used in parallel experiments.

These experiments demonstrate that both RK-20449 & Dasatinib are capable of depleting cancer cell ligation-induced SHP-1 activity by αTLR, an effect thereby unleashing macrophage proinflammatory response and antigen presentation.

In vivo tests. Following the hypothesis and in vitro findings, it is predicted that combination of RK-20449 or Dasatinib with proinflammatory activators would lead to intratumoral macrophage repolarization, phagocytosis and immunogenic antigen presentation that activates anti-tumor T cells. Three tumor models (MC38, KPC and LLC) that resist anti-PD-1/L1 and other therapies were tested with RK-20449 or Dasatinib combined with four proinflammatory modalities, i.e., TLR agonists, Sting activator, proinflammatory cytokines and tumor-focal RT.

FIG. 3A shows the experimental design. Three types of solid tumors were tested: 1) MC38 colorectal carcinoma, 2) KPC pancreatic ductal adenocarcinoma, and 3) LLC lung cancer. Tumors were subcutaneously engrafted (s.c.), and treatments started when palpable tumors formed >=200 mm³. Two treatment modalities included i) TK inhibitor (TKi, dark arrow), either RK-20449 or Dastinib (both at the dose of 5 mg/kg), and ii) αTLR, a mixture of CpG, PolyIC and R848 (each 20 μg). In most experiments, TKi and αTLR agonist were together suspended in 500 μl PBS and administered via s.c. at a location distant form tumor. The intermittent treatment scheme was designed to administrate the first cycle, 2× pulse administrations (d1 and d2), an intermittent period (3-7 days), followed by a second cycle treatment.

In tumors, our purpose is to target TAM, and through inhibition of TKI (e.g., by inhibiting Src family kinase (SFK)) to eliminate tumor therapies-induced ITIMs phosphorylation in macrophage iRs and thus prohibit SHP-1 activation. The result of this manipulation is to remove the master pathway of immunosuppression within the TME through iRs-SHP-1, thereby enabling TAM to be polarized towards proinflammatory activation.

Consequently, proinflammatory TAM drive TME inflammation and antigen presentation that activates T cell immunity against cancer. TKi may affect T cell function through inhibition of Lck and other TK within T cells that are required for T cell activation, proliferation and killing of cancer cells. For this consideration, TKi in treatment can be given in an intermittent manner, such as given for 2 days to unleash TAM for proinflammatory response and antigen presentation. Once the TME enters T cell mood, TKi treatment may be stopped to enable robust T cell activation and immunity against cancer. However, this method worked with continuous dosing as well.

FIG. 3B shows efficacy of TKi treatment against MC38 colorectal carcinoma. FIG. 3C shows efficacies of TKi treatment against KPC pancreatic ductal adenocarcinoma. FIG. 3D shows dose-dependent effect of TKi against LLC lung cancer. FIG. 3E show Dasatinib combination with various TLR agonists treating MC38 colorectal carcinoma. TKi such as Dasatinib moderately curbs down TLR or Sting agonists induced neutrophil infiltration into tumors, thus switching the TME response to cancer specific T cell infiltration. This phenomenon benefits tumor elimination by T cell immunity. FIG. 3G shows that inhibition of TK reduces tumor angiogenesis. TKi such as Dasatinib inhibits angiogenesis in tumors. This effect is through inhibition of VEGR, a receptor tyrosine kinase.

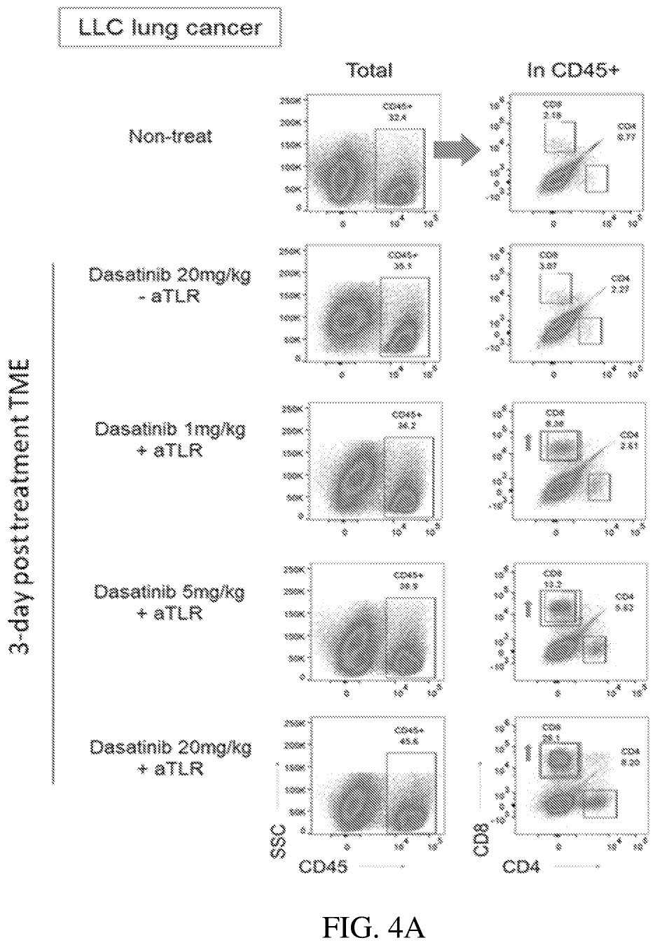

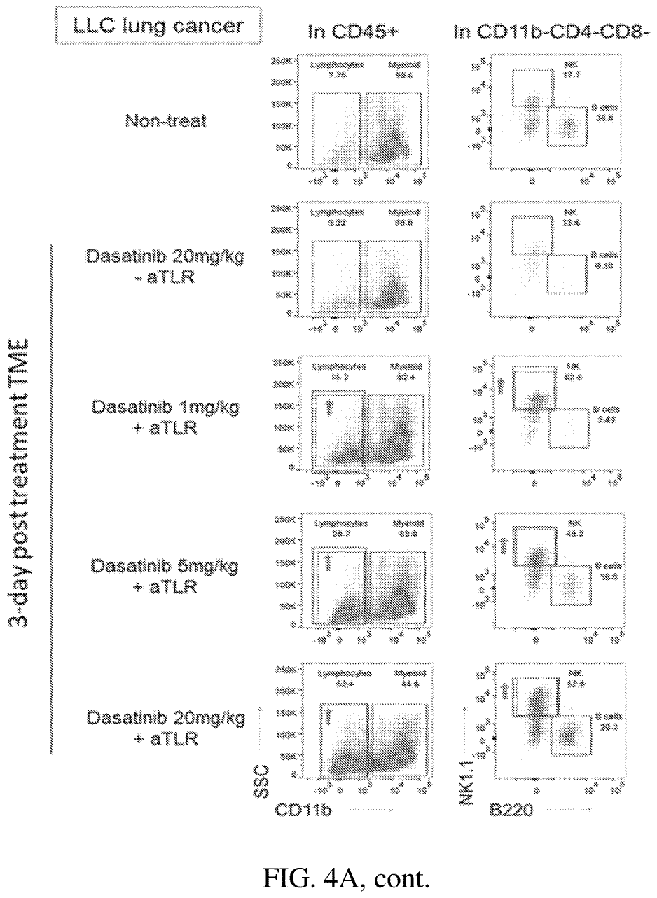

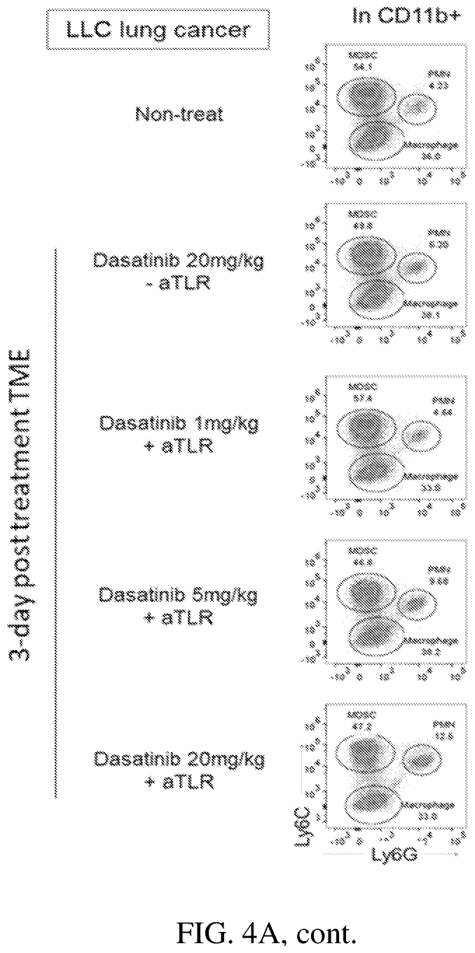

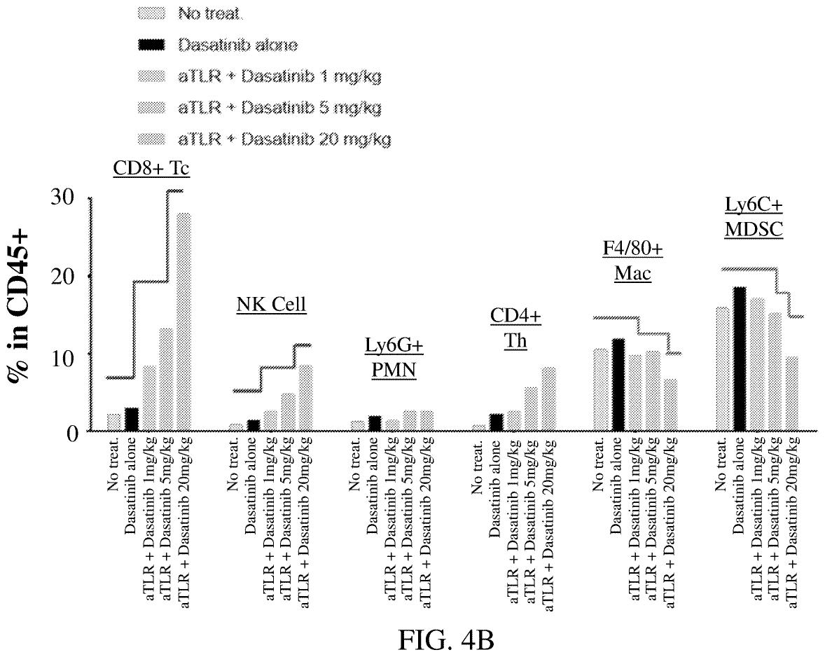

FIGS. 4A and 4B show Dasatinib and αTLR combination induced intratumoral T cell expansion. TME analyses of LLC tumors either without treatment (non-treat) or 3-day post treatment with Dasatinib alone, or Dasatinib at various doses plus αTLR were carried out. FIG. 4A shows flow cytometry analyses of the frequency of immune cells (CD45+) within the total cell population of tumor dissociates, as well as frequencies of individual immune cell types labeled by specific antibodies. FIG. 4B shows summary of each immune population dynamics. Dasatinib once combined with αTLR exhibited dose-dependent effect of elevating CD8+ T cells and NK cells and reducing macrophages and MDSC with TME.

FIGS. 5A-5C show efficacies of RK-20449 or Dasatinib combined with Sting activator (FIG. 5A), tumor-focal RT (FIG. 5B) or proinflammatory cytokines (FIG. 5C) for treating MC38 colorectal tumor. Mice with single engrafted MC38 tumor (s.c., >=200 mm³) were treated twice (d1 and d2) with RK-20449 or Dasatinib (each 20 mg/kg, s.c.) combined with either (FIG. 5A) Sting activator cGAMP (10 μg, i.p.) or (FIG. 5B) 8Gy tumor-focal RT, or (FIG. 5C) TNFα/IFNγ (each 20 μg, i.p.) or IFNγ/LPS (each 20 μg, i.p.). Tumor volume changes were recorded.

Note that the TLR agonists and STING activators shown and discussed above are representative of TLR agonists and STING activators tested. Tested TLR agonists include: LTA (TLR2), CpG (TLR3), PolyI; C (TLR9), LPS and MPLA (both TLR4), Flagellin (TLR5), multiple TLR7/8 activators including R848, Vesatolimod, Bropirimine, Motolimod, and Loxoribine; etc. STING activator tested include: MSA-2, ADU-S100 and cGAMP. Any one of these TLR agonists or STING activators, when combined with TKi for treating tumors, leading to tumor inhibition and/or regression.

FIGS. 6A-6C show that PD-1/PD-L1 immune checkpoint blockade bolsters efficacies of Dasatinib and αTLR combination therapy, accelerating tumor regression. FIG. 6A shows the experimental design. KPC tumors were subcutaneously engrafted into both flanks. After tumor formation, treatments were administrated with Dasatinib alone, Dasatinib plus αTLR, and Dasatinib plus αTLR and anti-PD-L1. Two pulse treatments were given in d1 and d2, followed by the second cycle three days later. FIG. 6B shows luminescence images showing tumor location and sizes. FIG. 6C shows tumor volume changes post-treatment. The tumor volumes of each flank were recorded daily and were averaged.

These experiments demonstrate that: 1) TKi such as RK-20449 or Dasatinib alone/monotherapy is not effective against tumor; 2) TKi combined with proinflammatory activators, e.g., TLR agonists, Sting activator, cytokines and RT, forms an effective therapeutic strategy, which is capable of inducing intratumoral proinflammatory polarization and T cell immunity, together achieving strong anti-tumor efficacies; 3) administration of with anti-PD-1/L1 immune checkpoint blockade further bolsters the efficacies of TKi and proinflammatory activator combination therapy, accelerating tumor regression.

### Example 2

Additional TK inhibitors were tested by the same in vitro assays to identify potential candidates that inhibit TK upstream of iRs, thereby capable of unleashing macrophage proinflammatory activation in a tumor milieu. Table 2 summarizes these tyrosine kinases inhibitors based upon the information vendor provided.

TABLE 2

| | Target | HCK | Src | Lck | Lyn | Abl |
|---|---|---|---|---|---|---|
| RK-20449 | IC₅₀ | 0.43 | 9 | <3 | <3 | 3000 |
| (HCK) | (nM) | | | | | |

| | Target | Bcr-Abl | Src | Lck | Yes | c-kit |
|---|---|---|---|---|---|---|
| Dasatinib | IC₅₀ | 1 | 0.5 | | 0.5 | 5 |
| | (nM) | | | | | |

| | Target | Abl | PDGFRα | VEGFR2 | Src |
|---|---|---|---|---|---|
| Ponatinib | IC₅₀ | 0.37 | 1.1 | 1.5 | 2.2 |
| | (nM) | | | | |

| | Target | Src | Abl |
|---|---|---|---|
| Bosutinib | IC₅₀ | 1.2 | 1 |
| | (nM) | | |

| | Target | Src | Lck | Yes | Lyn | Fyn | Fgr |
|---|---|---|---|---|---|---|---|
| Saracatinib | IC₅₀ | 2.7 | <4 | 4 | 5 | 10 | 10 |
| | (nM) | | | | | | |

| | Target | Src |
|---|---|---|
| KX2-391 | IC₅₀ | 20 |
| | (nM) | |

| | Target | c-Src | p38 |
|---|---|---|---|
| UM-164 | IC₅₀ | 2.7 | |
| | (nM) | | |

| | Target | Syk | Src | HCK |
|---|---|---|---|---|
| R406 | IC₅₀ | 41 | 20 | 90 |
| | (nM) | | | |

| | Target | Syk |
|---|---|---|
| Piceatannol | IC₅₀ | 10000 |
| | (nM) | |

In vitro assays. Macrophage response to αTLR was assayed in the presence of cancer cell ligation and with or without TK inhibitor. Macrophage SHP-1 activity (PTP activity), macrophage expression of antigen presentation machinery and proinflammatory cytokines were assayed.

Multiple tyrosine kinase inhibitors including those inhibit Src family tyrosine kinase, Syk, Able and Btk were tested for capability of diminishing macrophage SHP-1 activity induced by αTLR and cancer ligation (FIG. 7A), elevating antigen presentation machinery (FIG. 7B), and induction of proinflammatory cytokines (FIG. 7C). The same in vitro macrophage stimulation in the present of cancer cell ligation experimental system as in Example 1 was used.

As shown in FIGS. 7A-7C, UM164 and R406 were shown to have the capability of diminishing macrophage SHP-1 activity induced by αTLR and cancer cell ligation (FIG. 7A). Both UM164 and R406 also elevated macrophage expression of the antigen presentation machinery (FIG. 7B). Neither UM164 nor R406 enabled αTLR-induced proinflammatory cytokine production (FIG. 7C). These experiments identify UM-164 and R406 to be potential candidates, and these inhibitors are further tested in vivo for anti-tumor efficacy combined with proinflammatory stimuli.

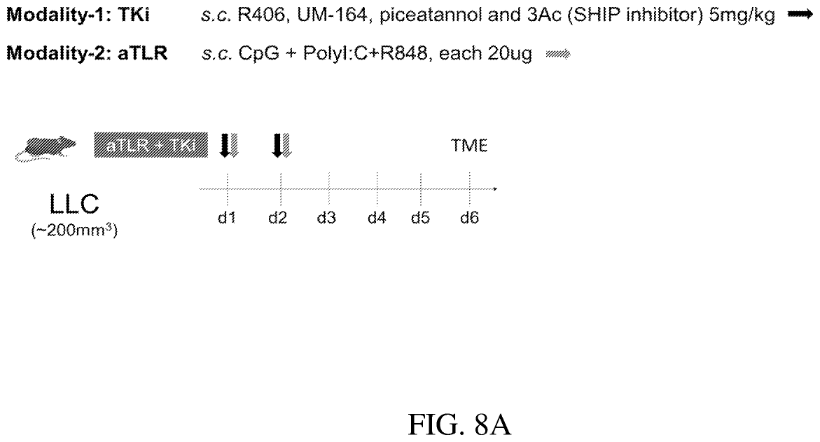

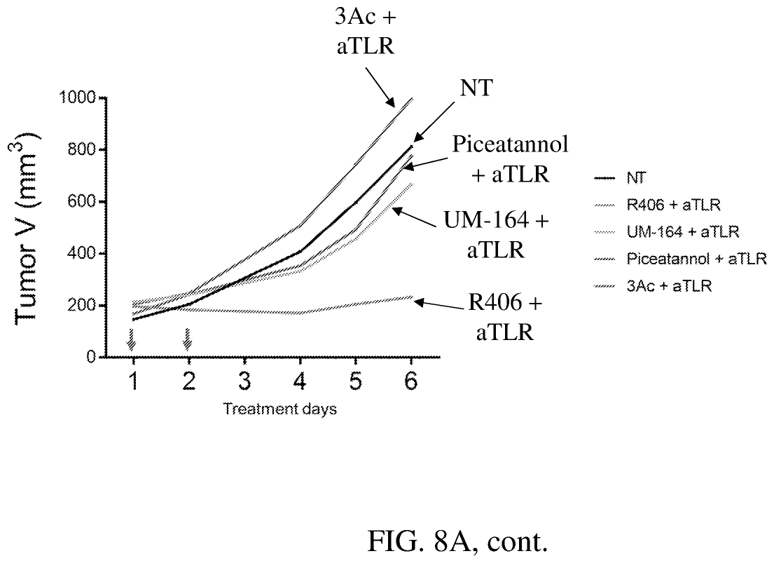

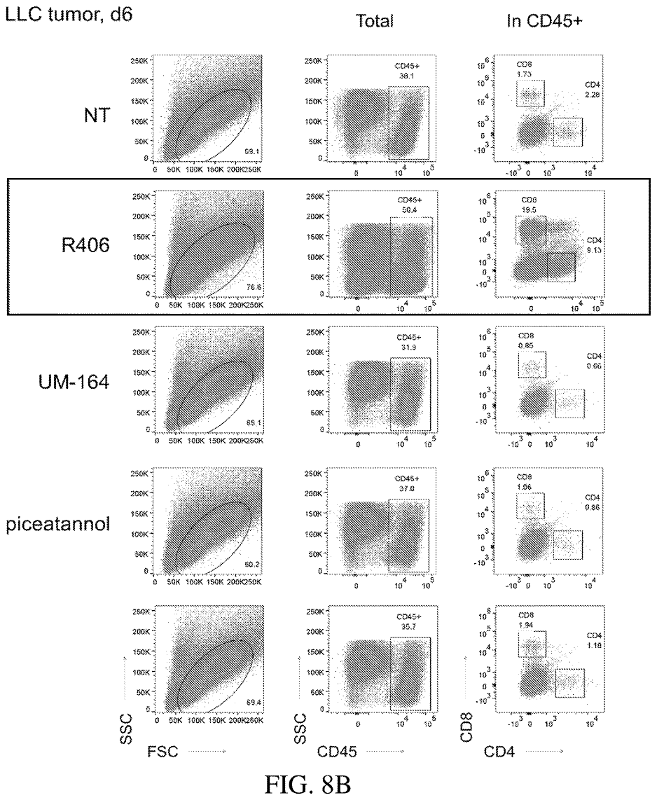

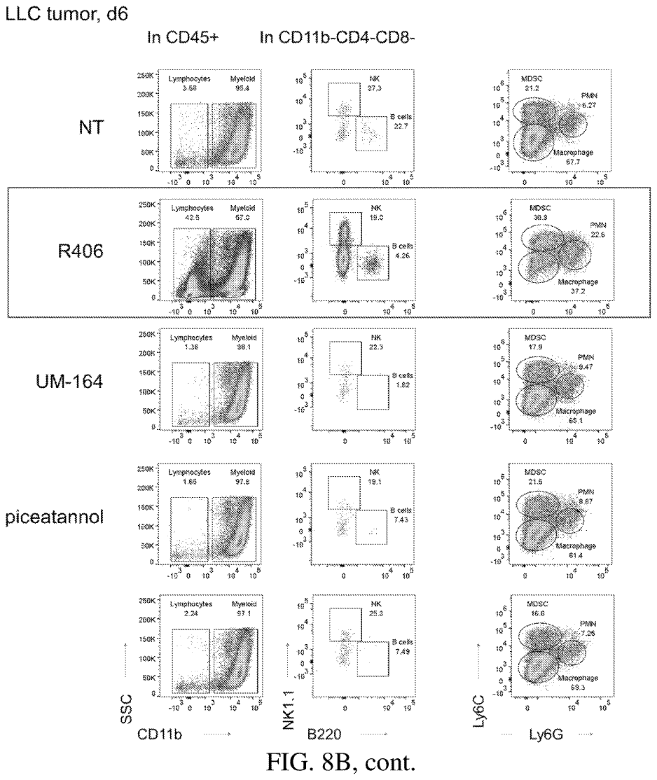

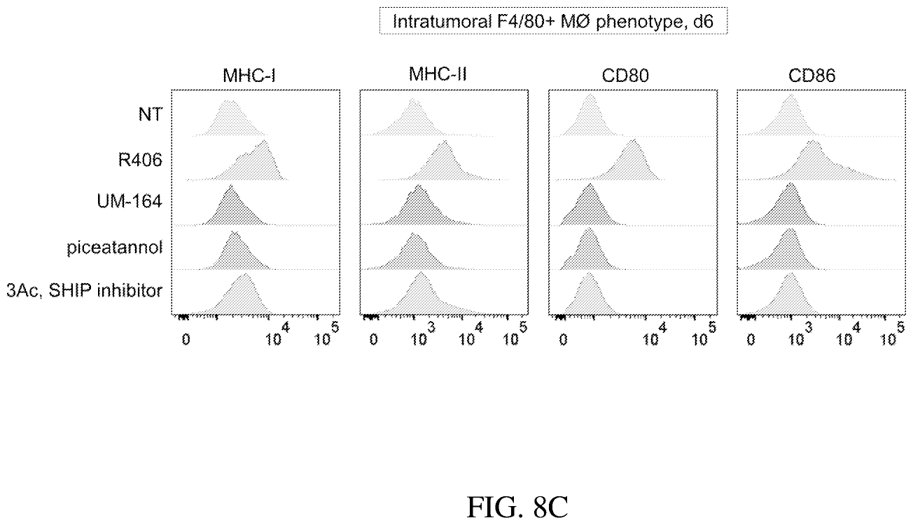

In vivo tests for anti-tumor efficacy. UM-164 and R406, as well as another Syk inhibitor Piceatannol and a SHIP inhibitor 3Ac, were combined with αTLR to treat established LLC tumor following the dosing regimen illustrated in FIG. 8A. As shown in FIG. 8A, R406, but not other inhibitors, combined with αTLR effectively suppressed LLC tumor. TME analyses show that R406 combined with αTLR induced intratumoral CD8+ T cell expansion. See FIG. 8B. R406 combined with αTLR induced intratumoral macrophages for antigen presentation.

Taken together, these experiments identified R406 to be an effective TK inhibitor that displayed anti-tumor efficacy once combined with αTLR.

### Example 3

Four tyrosine kinase (TK) inhibitors, Ponatinib (Bcr-Abl inhibitor), Bosutinib (Src inhibitor), Saracatinib (Src inhibitor) and KX2-391 (Src inhibitor), were also tested for unleashing TAM proinflammatory response and antigen presentation upon TLR stimulation, as well as in vivo for anti-tumor efficacy once combined with αTLR.

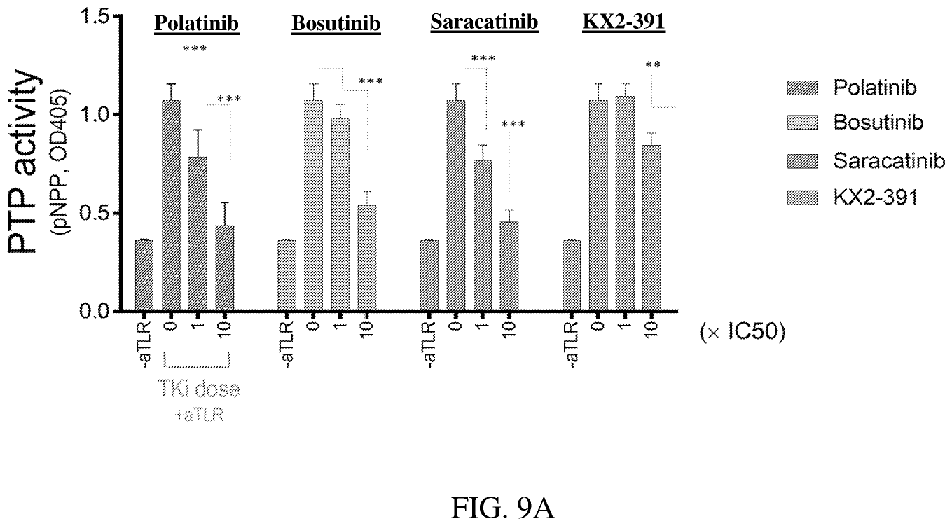

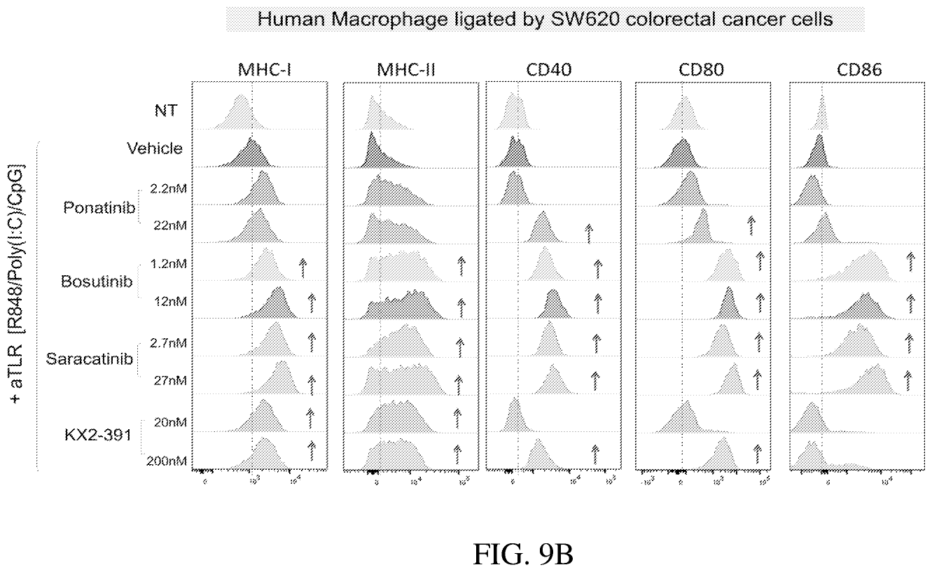

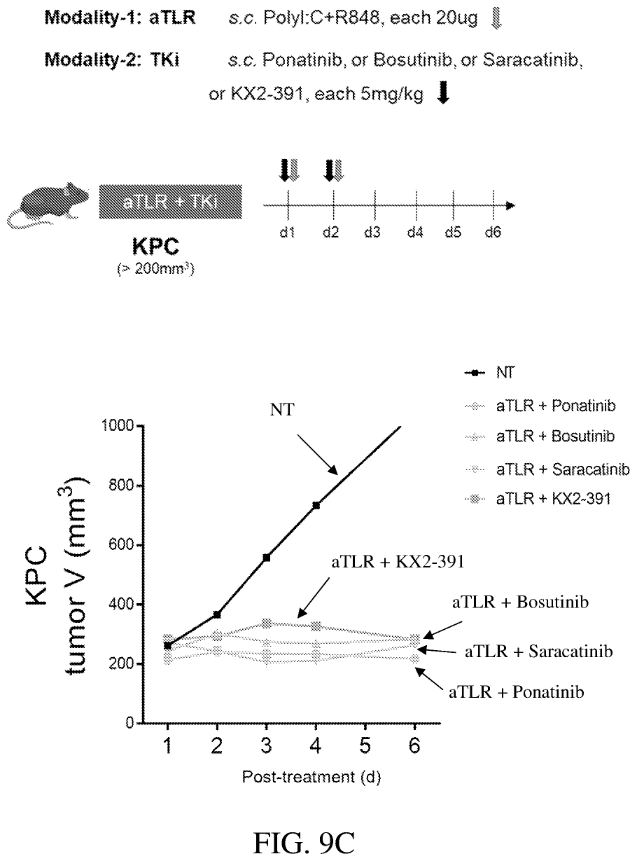

Specifically, an in vitro macrophage functional assay was set up in the presence of cancer cell (SW620) ligation of macrophage iRs. Four TK inhibitors including Ponatinib, Bosutinib, Saracatinib & KX2-391 were tested for capability of diminishing macrophage SHP-1 activity induced by αTLR and cancer cell ligation (FIG. 9A), and the ability of elevating antigen presentation molecule expression otherwise inhibited by cancer cell ligation (FIG. 9B). In vivo anti-tumor efficacies with the combination of αTLR were shown in (FIG. 9C).

As shown in FIGS. 9A-9C, all four TK inhibitors, once combined with TLR activator, are capable of triggering innate and adaptive immunity against cancer and displayed anti-tumor efficacy. In contrast, one of the four TK inhibitors exhibited strong anti-tumor efficacy when used as monotherapy. These results, including the above results, suggest that tyrosine kinases that inhibitor Src family kinases as shown in the Table 2 above, when combined with a proinflammatory therapy, turned on the TAM for innate and adaptive immunity and achieved anti-tumor efficacy.

### Example 4

Experiments were setup to test synergistic effects of inhibition TK and SHP-1 to treating solid tumors. Specifically, mice bearing KPC pancreatic adenocarcinoma (FIGS. 10A-10C) or MC38 colorectal carcinoma (FIGS. 10D-10E) were treated with αTLR plus TPI-1, αTLR plus Dasatinib, αTLR plus TPI-1 and Dasatinib, followed by recording tumor volume changes. The tumor microenvironments (TMEs) were analyzed for immune infiltrates in tumors on day 5 post treatments. See FIG. 10A for the experimental design testing treatment against KPC and FIG. 10D for experimental design testing treatment against MC38.

FIG. 10B shows KPC tumor volume changes following treatment with αTLR plus TPI-1, or αTLR plus TPI-1 and Dasatinib, versus tumors that received no treatment (NT) and exhibited continuous progression. FIG. 10C shows that, compared to the treatment with αTLR plus TPI-1, αTLR plus TPI-1 and Dasatinib further enhanced T cell immunity while reducing PMN infiltration, resulting in increased CD8 (Tc) and CD4 (Th) T cells, moderately increased NK cells, but reduced PMN in the TME followed treatment. FIG. 10E show MC38 tumor volume changes following treatment with αTLR plus TPI-1, αTLR plus Dasatinib, or αTLR plus TPI-1 and Dasatinib, versus tumors that received no treatment (NT) and exhibited continuous progression.

As demonstrated in FIGS. 10B, 10C, and 10E, in both KPC and MC38 tumor models, combination therapy with αTLR with the TK inhibitor Dasatinib and TPI-1 significantly enhanced anti-tumor efficacy than αTLR combined with Dasatinib or TPI-1 alone. These results demonstrated synergistic effects of inhibition of TK with Dasatinib and TPI-1.

### Example 5

This example demonstrates that neutralization of TNFα curbs down systemic inflammation without affecting anti-tumor efficacies by TKi, SHP-1 inhibition and αTLR combination.

Mice with established MC38 colorectal carcinoma (200-400 mm³) were treated with αTLR, TPI-1 and Dasatinib (s.c.), without or with additional treatment with anti-TNFα mAb or anti-IL-6 mAb (150 μg, i.p.). The treatment was repeated once (d1 and d2). Tumor volume changes were recorded, and tumor TMEs were analyzed for immune infiltrates on day 6 post treatments. See FIG. 11A.

As shown in FIG. 11B, tumor volume decreased following treatment of αTLR+TPI-1+Dasatinib, and the administration of anti-TNFα or anti-IL-6 did not interfere with their anti-tumor activities. Anti-TNFα mAb or anti-IL-6 mAb treatment also did not affect αTLR/TPI-1/Dasatinib therapy-induced increases in CD8 T cells (Tc) and NK cells, as well as reduction of macrophages and MDSC in the TME. See FIGS. 11C and 11D. Treating mice with anti-TNFα mAb, but not anti-IL-6 mAb, largely diminished the induction of inflammatory cytokines (TNFα, IL-6, IL-1β, IL-10, IFNα, and IFNγ) associated with the αTLR/TPI-1/Dasatinib combination therapy. Anti-TNFα treatment also markedly reduced monocyte and PMN chemokines CCL2, CCL5 and CXCL1 in circulation, while without reducing CXCL10 that is essential for T cell trafficking. FIG. 11E. Further, as shown in FIG. 11F, anti-TNFα treatment protected mice from developing splenomegaly and intestinal inflammation that were commonly associated with αTLR/TPI-1/Dasatinib therapy.

Together, the results showed that neither anti-TNFα nor anti-IL-6 interfered TKi/iShp1/αTLR for driving anti-tumor immunity or achieving therapeutic efficacies. Moreover, anti-TNFα exhibited beneficial effects by largely abrogating TKi/iShp1/αTLR-induced cytokine storm and systemic inflammation, thereby curbing down the therapy-associated adverse toxicity.

Moreover, although the above specific experiment involves using both TPI-1 and dasatinib and both polyI:C and R848, the results from experiments using either one of TPI-1 and dasatinib and either one of polyI:C and R848 achieved similar effects (data not shown). It was also found that the proper time window for anti-TNFα antibody treatment can be from at least a week prior (as long as the antibody is stable for the time window) to immediately after (e.g., within 0.5-1 hour) the SHP-1 inhibitor/αTLR treatment. It is preferable that the anti-TNFα antibody is provided prior to or simultaneously with the SHP-1 inhibitor and/or αTLR so that it maximally blocks the TNFα induced after the treatment of SHP-1 inhibitor and the pro-inflammatory agent.

1. A method of treating a cancer in an individual, comprising administering to the individual a) a tyrosine kinase inhibitor, and b) a pro-inflammatory agent.

2. The method of claim 1, wherein the method comprises systemically administering the tyrosine kinase inhibitor.

3. The method of claim 1, wherein the pro-inflammatory agent comprises an agent selected from the group consisting of a TLR agonist, a STING activator, a PAMP/DAMP activator, a chemotherapy, a pro-inflammatory cytokine, a cancer vaccine, an oncolytic virus, immune cells, a bacteria component, a sound treatment, a magnetic therapy, an electrical treatment, and an electrostatic treatment.

4. A method of treating a cancer in an individual, comprising administering to the individual a tyrosine kinase inhibitor, wherein the individual is under an inflammation reaction.

5. The method of claim 1, wherein the method further comprises administering the tyrosine kinase inhibitor to the individual intermittently.

6. The method of claim 1, wherein the tyrosine kinase inhibitor is an inhibitor of a tyrosine kinase of a Src family.

7. The method of claim 1, wherein the tyrosine kinase inhibitor inhibits any one or more of SRC, BLK, HCK, FYN, FGR and YES.

8. (canceled)

9. The method of claim 1, wherein the tyrosine kinase inhibitor inhibits SHP-1 signaling.

10-11. (canceled)

12. The method of claim 1, wherein the pro-inflammatory agent and the tyrosine kinase inhibitor are administered within 24 hours of each other.

13. (canceled)

14. The method of claim 1, wherein the pro-inflammatory agent comprises:
(1) a TLR agonist, wherein the TLR agonist comprises CpG, polyI:C, and/or R848;
(2) a bacteria component, wherein the bacteria component comprises lipopolysaccharide (LPS);
(3) a STING activator, wherein the STING activator comprises 2′3′-cGAMP;
(4) a chemotherapeutic agent, wherein the chemotherapy comprises azathioprine (AZA);
(5) a pro-inflammatory cytokine, wherein the pro-inflammatory cytokine comprises IL-1b, IL-18, IL-6, and/or TNFα;
(6) a radiation therapy, wherein the radiation therapy comprises a dose of the radiation therapy that is insufficient to kill tumor cells; and/or
(7) a checkpoint inhibitor, wherein the checkpoint inhibitor comprises an anti-PD-L1 antibody, an anti-PD-1 antibody or an anti-CLTA4 antibody.

15-20. (canceled)

21. The method of claim 1, wherein the pro-inflammatory agent and the tyrosine kinase inhibitor are administered simultaneously or concurrently.

22. The method of claim 1, wherein the pro-inflammatory agent comprises immune cells.

23. The method of claim 4, where the method further comprises administration of immune cells wherein:
a) the immune cells comprise or are macrophages;
b) the immune cells are derived from monocytes;
c) the immune cells express a high level of MHC-I, MHC-II, CD80 and/or CD86;
d) the immune cells express one or more pro-inflammatory cytokines;
e) the immune cells do not express a significant level of TGFβ and/or IL-10;
f) the immune cells comprise T cells;
g) the immune cells are engineered to express a chimeric antigen receptor; and/or
h) the tyrosine kinase inhibitor and the immune cells are administered within 24 hours of each other.

24. The method of claim 1, further comprising administering to the individual an effective amount of a SHP-1 inhibitor.

25. The method of claim 1, further comprising administering to the individual an effective amount of an anti-TNFα antibody.

26. The method of claim 1, wherein the cancer is a solid tumor.

27. (canceled)

28. The method of claim 1, wherein the cancer is a late-stage cancer.

29. The method of claim 1, wherein the cancer is resistant or refractory to a radiation therapy, a chemotherapeutic agent, and/or a checkpoint inhibitor.

30. The method of claim 1, wherein the individual is a human.

31. A composition comprising a tyrosine kinase inhibitor and a pro-inflammatory agent, wherein the pro-inflammatory agent comprises an agent selected from the group consisting of immune cells, a TLR agonist, a STING activator, an agent used in radiation therapy, a PAMP/DAMP activator, a checkpoint inhibitor, a pro-inflammatory cytokine, a chemotherapeutic agent, a bacteria component, a cancer vaccine, an oncolytic virus, and an agent used in sound treatment, a magnetic therapy, an electrical treatment or an electrostatic treatment.

32. (canceled)
